(19) United States
(12) Patent Application Publication
(10) Pub. No.: US 2026/0162821 A1
(43) Pub. Date: Jun. 11, 2026

(54) BEHAVIOR CONTROL SYSTEM, CONTROL DEVICE, ELECTRONIC DEVICE, AND AVATAR DISPLAY DEVICE

(71) Applicant: SoftBank Group Corp., Tokyo (JP)

(72) Inventor: Masayoshi SON, Tokyo (JP)

(73) Assignee: SoftBank Group Corp., Tokyo (JP)

(21) Appl. No.: 19/538,071

(22) Filed: Feb. 12, 2026

Related U.S. Application Data
(63) Continuation of application No. PCT/JP2024/031204, filed on Aug. 30, 2024.

(30) Foreign Application Priority Data
Aug. 30, 2023 (JP) 2023-139944
Sep. 7, 2023 (JP) 2023-145288
Sep. 14, 2023 (JP) 2023-149395
Sep. 21, 2023 (JP) 2023-155595
Sep. 21, 2023 (JP) 2023-155596
Sep. 21, 2023 (JP) 2023-155682
Sep. 21, 2023 (JP) 2023-155799
Sep. 21, 2023 (JP) 2023-155927
Sep. 21, 2023 (JP) 2023-155928
Sep. 25, 2023 (JP) 2023-161536
Sep. 26, 2023 (JP) 2023-163441
Sep. 26, 2023 (JP) 2023-163754
Sep. 27, 2023 (JP) 2023-164818
Sep. 27, 2023 (JP) 2023-165904
Sep. 27, 2023 (JP) 2023-165932
Sep. 27, 2023 (JP) 2023-166356
Sep. 28, 2023 (JP) 2023-167671
Sep. 28, 2023 (JP) 2023-167792
Sep. 28, 2023 (JP) 2023-168570
Sep. 29, 2023 (JP) 2023-169768

(51) Int. Cl. G16H 40/67; G06F 3/01; G06F 21/62; G06T 13/00; G06V 10/40; G06V 10/75; G06V 40/16; G16H 20/70; H04L 9/40

(52) U.S. Cl. CPC G16H 40/67; G06F 3/011; G06F 21/6245; G06T 13/00; G06V 10/751; G06V 40/168; G06V 40/172; G16H 20/70; H04L 63/0861; G06F 2203/011; G06V 10/40

(57) ABSTRACT

A behavior control system according to an embodiment of the present invention recognizes the behavior of an artist and determines the behavior of an avatar corresponding to the recognized behavior of the artist. Then, the behavior control system controls the avatar on the basis of the determined behavior of the avatar.

### CROSS-REFERENCE TO RELATED APPLICATIONS

This application is a continuation of International Application No. PCT/JP2024/031204, filed on Aug. 30, 2024, which claims priority from Japanese Patent Application No. 2023-139944, filed on Aug. 30, 2023, Japanese Patent Application No. 2023-145288, filed on Sep. 7, 2023, Japanese Patent Application No. 2023-149395, filed on Sep. 14, 2023, Japanese Patent Application No. 2023-155595, filed on Sep. 21, 2023, Japanese Patent Application No. 2023-155596, filed on Sep. 21, 2023, Japanese Patent Application No. 2023-155682, filed on Sep. 21, 2023, Japanese Patent Application No. 2023-155799, filed on Sep. 21, 2023, Japanese Patent Application No. 2023-155927, filed on Sep. 21, 2023, Japanese Patent Application No. 2023-155928, filed on Sep. 21, 2023, Japanese Patent Application No. 2023-161536, filed on Sep. 25, 2023, Japanese Patent Application No. 2023-163441, filed on Sep. 26, 2023, Japanese Patent Application No. 2023-163754, filed on Sep. 26, 2023, Japanese Patent Application No. 2023-164818, filed on Sep. 27, 2023, Japanese Patent Application No. 2023-165904, filed on Sep. 27, 2023, Japanese Patent Application No. 2023-165932, filed on Sep. 27, 2023, Japanese Patent Application No. 2023-166356, filed on Sep. 27, 2023, Japanese Patent Application No. 2023-167671, filed on Sep. 28, 2023, Japanese Patent Application No. 2023-167792, filed on Sep. 28, 2023, Japanese Patent Application No. 2023-168570, filed on Sep. 28, 2023, Japanese Patent Application No. 2023-169768, filed on Sep. 29, 2023. The entire disclosure of each of the above applications is incorporated herein by reference.

### BACKGROUND OF THE INVENTION

### Field of the Invention

The present invention relates to a behavior control system, a control device, an electronic device, and an avatar display device.

### Description of the Related Art

Patent Document 1 discloses a technology for determining appropriate actions of a robot in response to a user's state. In the conventional technology of Patent Document 1, when the robot recognizes a user's reaction to a specific action performed by the robot and cannot determine the robot's action in response to the recognized user's reaction, the robot updates a robot's action by receiving information from a server regarding actions suitable for the recognized user's state.
[Patent Document 1] Japanese Patent No. 6053847

However, in the conventional technology, there are cases where it is not possible to output appropriate information according to user's emotions.

### SUMMARY OF THE INVENTION

According to a first aspect of the present invention, there is provided a behavior determination unit configured to recognize actions of an artist and determine the actions of an avatar corresponding to the recognized actions of the artist; and a behavior control unit configured to control the avatar based on the actions of the avatar determined by the behavior determination unit.

According to one aspect of embodiments, it is possible to output appropriate information according to user's emotions.

### BRIEF DESCRIPTION OF THE DRAWINGS

FIG. 1 is a diagram schematically illustrating an example of a system according to a first embodiment.

FIG. 2 is a diagram schematically illustrating a functional configuration of a control device for controlling an avatar.

FIG. 3 is a diagram schematically illustrating a data structure of character data.

FIG. 4 is a diagram schematically illustrating an example of an operation flow related to character setting.

FIG. 5 is a diagram schematically illustrating an example of an operation flow related to determining behaviors of an avatar.

FIG. 6 is a diagram schematically illustrating an example of a hardware configuration of a computer functioning as a control device and a server.

FIG. 7 is a diagram illustrating an emotion map in which multiple emotions are mapped.

FIG. 8 is a diagram illustrating another example of the emotion map.

FIG. 9 is a diagram illustrating an example of an emotion table.

FIG. 10 is a diagram illustrating an example of an emotion table.

FIG. 11 is a diagram schematically illustrating a functional configuration of a control device.

FIG. 12 is a diagram schematically illustrating an example of an operation flow related to determining the behavior of an avatar.

FIG. 13 is a diagram schematically illustrating a functional configuration of a support unit.

FIG. 14 is a diagram schematically illustrating an example of an operation flow by the support unit.

FIG. 15 is a diagram schematically illustrating a data structure of character data.

FIG. 16 is a diagram schematically illustrating a functional configuration of an output unit.

FIG. 17 is a diagram schematically illustrating an example of an operation flow by the output unit.

FIG. 18 is a diagram schematically illustrating a functional configuration of the output unit.

FIG. 19 is a diagram schematically illustrating an example of an operation flow by the output unit.

FIG. 20 is a diagram schematically illustrating a functional configuration of a control device for controlling an avatar.

FIG. 21 is a diagram schematically illustrating an example of an operation flow related to determining behaviors of an avatar.

FIG. 22 is a diagram schematically illustrating a functional configuration of the output unit.

FIG. 23 is a diagram schematically illustrating an example of an operation flow by the output unit.

FIG. 24 is a diagram schematically illustrating a functional configuration of the output unit.

FIG. 25 is a diagram schematically illustrating an example of an operation flow by the output unit.

FIG. 26 is a diagram schematically illustrating a functional configuration of the output unit.

FIG. 27 is a diagram schematically illustrating an example of an operation flow by the output unit.

FIG. 28 is a diagram schematically illustrating a functional configuration of the output unit.

FIG. 29 is a diagram schematically illustrating an example of an operation flow by the output unit.

FIG. 30 is a diagram schematically illustrating an example of an operation flow by the support unit.

FIG. 31 is a diagram schematically illustrating an example of an operation flow by the output unit.

FIG. 32 is a diagram schematically illustrating an example of an operation flow by the output unit.

FIG. 33 is a diagram schematically illustrating a functional configuration of the control device for controlling the avatar.

FIG. 34 is a diagram schematically illustrating a functional configuration of an event detection unit.

FIG. 35 is a diagram schematically illustrating an example of an operation flow by the event detection unit.

FIG. 36 is a diagram schematically illustrating an example of a system according to a fifteenth embodiment.

FIG. 37 is a diagram schematically illustrating a functional configuration of the control device for controlling the avatar.

FIG. 38 is a diagram schematically illustrating an example of an operation flow related to determining behaviors of an avatar.

FIG. 39 is a diagram schematically illustrating an example of a control system according to a sixteenth embodiment.

FIG. 40 is a diagram schematically illustrating a functional configuration of the control device for controlling the avatar.

FIG. 41 is a diagram schematically illustrating an example of an operation flow related to determining behaviors of an avatar in the control device.

FIG. 42 is a diagram schematically illustrating an example of a control system according to a nineteenth embodiment.

FIG. 43 is a diagram schematically illustrating a functional configuration of the control device.

FIG. 44 is a diagram schematically illustrating an example of a control system according to a twentieth embodiment.

FIG. 45 is a diagram schematically illustrating a functional configuration of the control device for controlling the avatar.

FIG. 46 is a diagram schematically illustrating an example of an operation flow related to determining behaviors of an avatar.

### DETAILED DESCRIPTION OF THE PREFERRED EMBODIMENTS

### Example 1 of Embodiment

Hereinafter, the present invention will be described through embodiments of the invention, but the following embodiments do not limit the invention according to the scope of the claims. In addition, not all combinations of features described in the embodiments are necessarily essential as solutions to the invention.

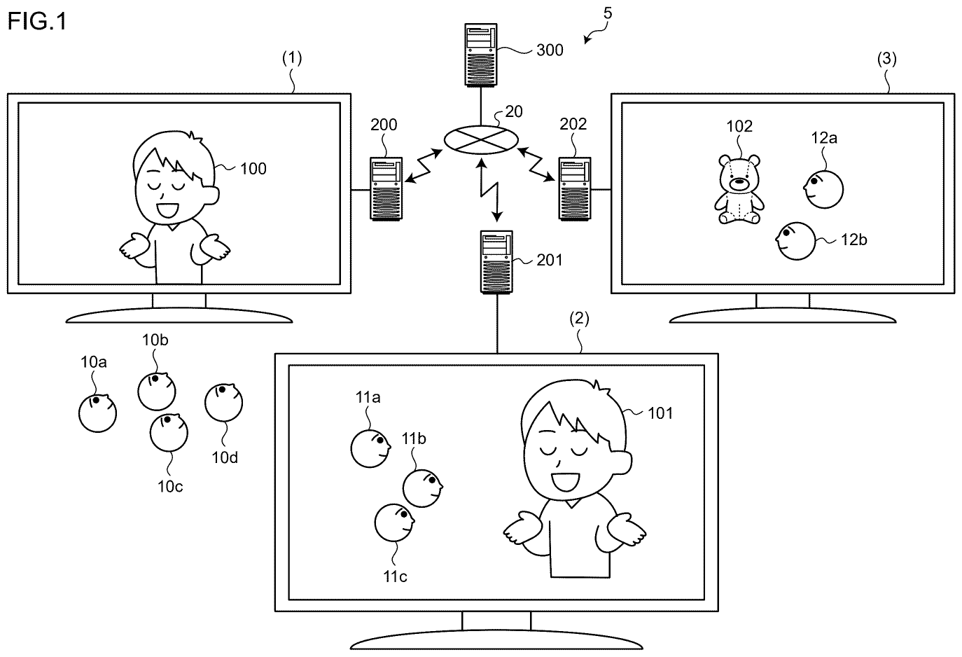

FIG. 1 is a diagram schematically illustrating an example of a system 5 according to the present embodiment. The system 5 includes control devices 200 to 202 for controlling avatars such as avatar 100, avatar 101, avatar 102; and a server 300. The control device 200 for controlling the avatar 100 is wirelessly or wiredly connected to a display device such as a PC monitor or a smartphone display, or to a predetermined wearable terminal device related to at least one of augmented reality, virtual reality, or mixed reality. The control device 200 is equipped with various input/output devices such as a speaker for outputting sounds like voice, a microphone for detecting sounds like voice, and a camera.

Users 10a, 10b, 10c, and 10d are users who communicate with the avatar 100. Users 11a, 11b, and 11c are users who communicate with the avatar 101. Users 12a and 12b are users who communicate with the avatar 102. In the description of this embodiment, the users 10a, 10b, 10c, and 10d may be collectively referred to as the users 10. Similarly, the users 11a, 11b, and 11c may be collectively referred to as the users 11, and the users 12a and 12b as users 12. The avatar 101 and the control device 201, and the avatar 102 and the control device 202, have substantially the same functions as the avatar 100 and the control device 200. Therefore, the system 5 will be described mainly focusing on the functions of the avatar 100.

As shown in FIG. 1, the avatars 100 to 102 controlled by the control devices 200 to 202 communicate with the users 10 to 12 based on various forms. For example, as shown in (1) of FIG. 1, the avatar 100 is represented as a person or the like displayed on a display device, and communicates with the user 10 via input/output devices provided in the control device 200. Also, as shown in (2) of FIG. 1, the avatar 101 communicates with the user 11, who is displayed as a person or character on a display device, in a virtual space. Further, as shown in (3) of FIG. 1, the avatar 102 can be displayed as a character rather than a person, and communicates with the user 12a as the character. Note that, regarding the display device described above, in FIG. 1, a display monitor is described as an example, but it is not limited thereto. For example, in this embodiment, the display device may include monitors, display units of notebook PCs or mobile terminals, wearable terminal devices related to augmented reality, virtual reality, or mixed reality, such as VR/AR goggles, VR/AR glasses, and other wearable devices that provide information perceptible by the user's five senses.

The appearance of the avatar may be human-like, as in the avatar 100 and the avatar 101, or may be a character, as in the avatar 102. For example, the avatars 100 and 101 may provide counseling by having an avatar dressed as a counselor nod and listen like a real counselor, thereby providing business persons with labor issues with emotional support and appropriate coping methods or self-care methods for workplace concerns and stress. For example, the avatar 102, by having a character-like appearance, is considered to be particularly familiar to children.

Avatar 100 is controlled by the control device 200 and communicates with the user 10 or provides video to the user 10 via input/output devices provided in the control device 200. At this time, the control device 200, in cooperation with the server 300 or the like communicable via a communication network 20, provides conversation between the avatar 100 and the user 10, or provides video and the like to the user 10. For example, the control device 200 not only learns appropriate conversation by itself, but also learns in cooperation with a server 300 to enable more appropriate conversation with the user 10. In addition, the control device 200 may have the server 300 record video data of the user 10 and the like, and request video data and the like from the server 300 as needed to provide to the user 10.

The control device 200 also stores emotion values representing the types of emotions of the avatar 100. For example, the control device 200 stores emotion values representing the intensity of each emotion such as “joy,” “anger,” “sorrow,” “pleasure,” “comfort,” “discomfort,” “relief,” “anxiety,” “sadness,” “excitement,” “worry,” “ease,” “fulfillment,” “emptiness,” and “normal” as the emotion values of the avatar 100. For example, when the emotion value of excitement is high, the control device 200 controls the avatar 100 to speak at a high speed during conversation with the user 10. In this way, the avatar 100 can express its own emotions through actions by control of the control device 200.

The control device 200 may be configured to determine the behavior of the avatar 100 corresponding to the emotions of the user 10 by matching a text generation model (so-called AI (Artificial Intelligence) chat engine) with an emotion engine. Specifically, the control device 200 may be configured to recognize the behavior of the user 10, determine the emotion of the user 10 for the behavior, and determine the behavior of the avatar 100 corresponding to the determined emotion.

More specifically, when the control device 200 recognizes the behavior of the user 10, it automatically generates the behavior content that the avatar 100 should take in response to the behavior of the user 10 using a preset text generation model. The text generation model may be interpreted as an algorithm and computation for automatic dialogue processing by text. The text generation model is known, for example, as disclosed in Japanese Unexamined Patent Application Publication No. 2018-081444, and thus detailed description is omitted. Such a text generation model is configured by a large language model (LLM: Large Language Model). Thus, in this embodiment, by combining a large language model and an emotion engine, it is possible to reflect the emotions of the user 10 and the avatar 100, as well as various language information, in the behavior of the avatar 100. That is, according to this embodiment, a synergistic effect can be obtained by combining a text generation model and an emotion engine.

The control device 200 recognizes the actions of an artist and determines the actions of the avatar 100 corresponding to the recognized actions of the artist. The control device 200 controls the avatar based on the determined actions of the avatar.

An artist is a person engaged in creative activities. An artist includes a person who creates or produces works of art. For example, an artist includes sculptors, painters, directors, and writers. In addition, an artist includes performers and musicians.

The control device 200 determines actions related to the artist's performance. The control device 200 also determines actions related to the artist's creativity. The control device 200 further determines actions related to the artist's expressiveness. In the following sections, the user 10 is described as an artist, but the invention is not limited thereto; for example, the avatar 100 may be the artist.

The control device 200 also has a function to recognize the behavior (state) of the user 10. The control device 200 recognizes the behavior of the user 10 by analyzing the facial image of the user 10 acquired by a camera function and the voice of the user 10 acquired by a microphone function. Based on the recognized behavior of the user 10, the control device 200 determines the behavior to be executed by the avatar 100.

The control device 200 stores rules that define the behavior to be executed by the avatar 100 based on the emotions of the user 10, the emotions of the avatar 100 itself, and the behavior of the user 10, and performs various actions according to the rules.

Specifically, the control device 200 stores response rules for determining the behavior of the avatar 100 based on the emotions of the user 10, the emotions of the avatar 100, and the behavior of the user 10. For example, the response rules specify that when the behavior of the user 10 is “laughing,” the behavior of the avatar 100 is also “laughing.” The response rules also specify that when the behavior of the user 10 is “angry,” the behavior of the avatar 100 is “apologizing.” The response rules further specify that when the behavior of the user 10 is “asking a question,” the behavior of the avatar 100 is “answering.” The response rules also specify that when the behavior of the user 10 is “sad,” the behavior of the avatar 100 is “speaking to” the user.

Based on the response rules, when the control device 200 recognizes that the behavior of the user 10 is “angry,” it selects the action of “apologizing” as the action to be executed by the avatar 100, as specified in the response rules. For example, when the avatar 100 selects the action of “apologizing,” it performs an apologizing gesture and outputs a voice expressing apologetic words.

In addition, it is specified that when the emotion of the avatar 100 is “normal” (that is, “joy”=0, “anger”=0, “sorrow”=0, “pleasure”=0) and the state of the user 10 is “alone and seems lonely,” the emotion of the avatar 100 changes to “worry” and the action of “speaking to” can be executed.

Based on the response rules, when the control device 200 recognizes that the current emotion of the avatar 100 is “normal” and the user 10 is alone and seems lonely, it increases the “sorrow” emotion value of the avatar 100. The control device 200 also selects the action of “speaking to” as the action to be executed by the avatar 100 toward the user 10, as specified in the response rules. For example, when the control device 200 selects the action of “speaking to,” it outputs the phrase “What's wrong?” in a worried voice of the avatar 100, expressing concern. In this way, the behavior of the avatar 100 is determined according to, for example, emotions and actions of the user 10, emotions of the avatar 100, and emotion values thereof.

The control device 200 also transmits user reaction information indicating that a positive reaction was obtained from the user 10 as a result of this action to the server 300. The user reaction information includes, for example, the user action of “angry,” the action of the avatar 100 of “apologizing,” the fact that the reaction of the user 10 was positive, and attributes of the user 10. The user reaction information may also include negative reactions.

The control device 200 stores user reaction information regarding user's reaction to the avatar 100. The server 300 receives and stores user reaction information not only from the control device 200 that controls the avatar 100, but also from the control devices 201 and 202 that control the avatar 101 and the avatar 102, respectively. The server 300 analyzes user reaction information from the control devices 200, 201, and 202 and updates the response rules. That is, the server 300 analyzes user reaction information from other control devices and updates the response rules.

The control device 200 may inquire the server 300 for updated response rules and receive the updated response rules from the server 300. The control device 200 incorporates the updated response rules into response rules stored in the control device 200. Thus, the control device 200 can incorporate response rules acquired by the control devices 201 and 202 for the avatars 101 and 102, etc., into its own response rules. When the response rules are updated, the updated response rules may be automatically transmitted from the server 300 to the control device 200.

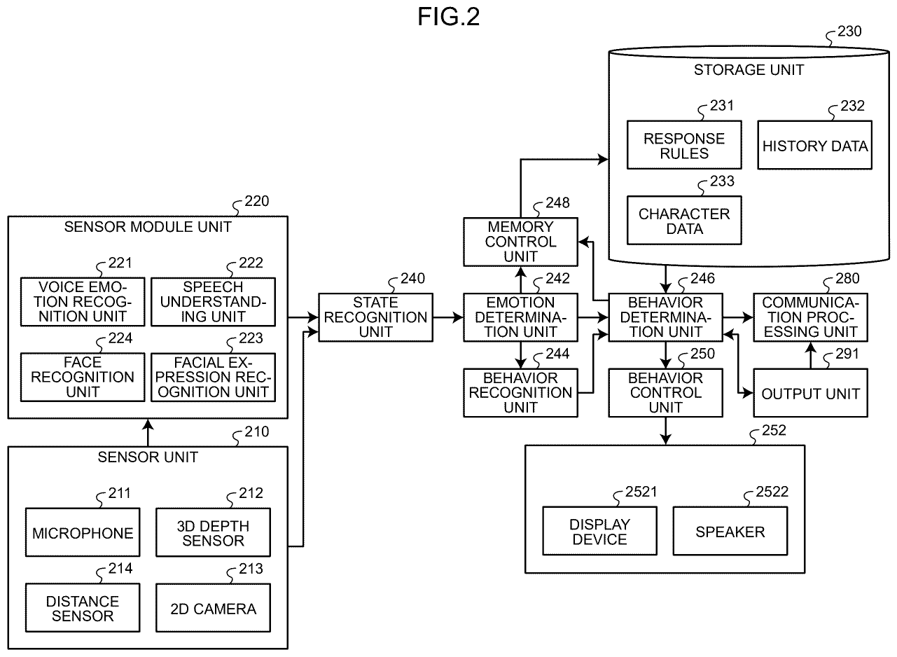

FIG. 2 is a diagram schematically illustrating a functional configuration of the control device 200. The control device 200 includes a sensor unit 210, a sensor module unit 220, a storage unit 230, a state recognition unit 240, an emotion determination unit 242, a behavior recognition unit 244, a behavior determination unit 246, a memory control unit 248, a behavior control unit 250, a controlled object 252, a communication processing unit 280, and an output unit 291.

The controlled object 252 includes a display device 2521 and a speaker 2522, among others. The display device 2521 displays the avatar 100 itself and images related to the avatar 100. The speaker 2522 outputs sounds related to conversation and actions of the avatar 100.

The sensor unit 210 includes a microphone 211, a 3D depth sensor 212, a 2D camera 213, and a distance sensor 214. The microphone 211 continuously detects audio and outputs audio data. The microphone 211 may be provided in a dummy head or simulator and may have a function for binaural recording. The 3D depth sensor 212 continuously emits an infrared pattern and analyzes the infrared pattern from infrared images continuously captured by an infrared camera to detect contours of objects. The 2D camera 213 is an example of an image sensor. The 2D camera 213 captures images using visible light and generates visible image information. The distance sensor 214 detects the distance to an object by emitting, for example, laser or ultrasound. The sensor unit 210 may also include, in addition, a thermography sensor, clock, gyro sensor, touch sensor, motor feedback sensor, and others.

The storage unit 230 includes response rules 231, history data 232, and character data 233. The history data 232 includes user's past emotion values and behavior history. These emotion values and behavior history are recorded for each of the users 10 by associating it with identification information of the corresponding user 10. At least a part of the storage unit 230 is implemented by a storage medium such as memory. It may also include a person database (namely, person DB) that stores face images and attribute information of the users 10. Among the components of the control device 200 shown in FIG. 2, functions of components other than the controlled object 252, sensor unit 210, and storage unit 230 can be realized by a CPU operating based on a program. For example, functions of these components can be implemented as CPU operations by basic software (OS) and programs running on the OS.

The character data 233 is data associating characters with ages. For example, a character may be a person appearing in existing content such as animation, video games, manga, or movies. The character may also be an animal or plant with a personality, or an inanimate object (such as a robot).

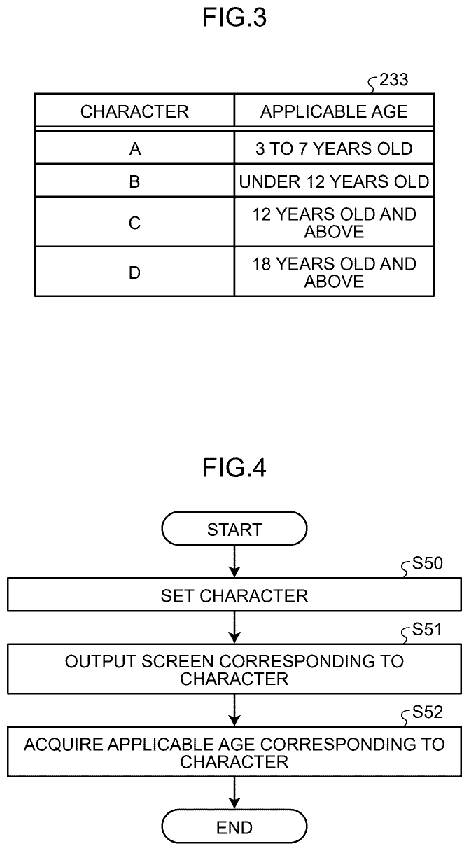

For example, an age (target age) associated with a character in the character data 233 is determined based on an age group of viewers assumed as a target for a content in which the character appears. FIG. 3 is a diagram schematically illustrating the data structure of the character data 233.

For example, suppose character “A” appears in an animation for kindergarten children. In this case, as shown in FIG. 3, a target age associated with character “A” is “3 to 7 years old.”

Also, for example, suppose a movie in which character “C” appears contains violent scenes and is not suitable for viewing by infants. In this case, as shown in FIG. 3, the target age associated with character “C” is “12 years old and above.”

An age in the character data 233 may be determined based on age ratings by rating organizations such as Pan European Game Information (PEGI), the Film Classification and Rating Organization, or the Computer Entertainment Rating Organization (CERO). The target age may be defined by a range such as “3 to 5 years old” or “12 years old and above,” or by a single value such as “10 years old” or “15 years old.”

The sensor module unit 220 includes a voice emotion recognition unit 221, a speech understanding unit 222, a facial expression recognition unit 223, and a face recognition unit 224. Information detected by the sensor unit 210 is input to the sensor module unit 220. The sensor module unit 220 analyzes the information detected by the sensor unit 210 and outputs the analysis results to the state recognition unit 240.

The voice emotion recognition unit 221 of the sensor module unit 220 analyzes voice of the user 10 detected by the microphone 211 and recognizes the emotion of the user 10. For example, the voice emotion recognition unit 221 extracts features such as frequency components of the voice and recognizes the emotion of the user 10 based on the extracted features. The speech understanding unit 222 analyzes voice of the user 10 detected by the microphone 211 and outputs character information representing contents of the user 10's speech.

The facial expression recognition unit 223 recognizes facial expression and emotion of the user 10 from images of the user 10 captured by the 2D camera 213. For example, the facial expression recognition unit 223 recognizes facial expression and emotion of the user 10 based on shapes and positional relationship of eyes and mouth.

The face recognition unit 224 recognizes a face of the user 10. The face recognition unit 224 recognizes the user 10 by matching a face image stored in the person DB (not shown) with a face image of the user 10 captured by the 2D camera 213.

The state recognition unit 240 recognizes a state of the user 10 based on information analyzed by the sensor module unit 220. For example, the state recognition unit 240 uses analysis results of the sensor module unit 220 to mainly perform processing related to perception. For example, the state recognition unit 240 generates perceptual information such as “The user is alone” or “There is a 90% probability that the user is not smiling.” The state recognition unit 240 performs processing to understand the meaning of the generated perceptual information. For example, the state recognition unit 240 generates semantic information such as “The user is alone and seems lonely.” In addition, for example, the state recognition unit 240 generates perceptual information such as “Dad is alone” or “There is a 90% probability that dad is not smiling.” For example, the state recognition unit 240 generates semantic information such as “Dad is alone and seems lonely.”

The state recognition unit 240 may also mainly perform processing related to perception using analysis results of the sensor module unit 220. For example, the state recognition unit 240 generates perceptual information such as “There is a 90% probability that the user is not smiling.” The state recognition unit 240 also performs processing to understand the meaning of the generated perceptual information. For example, the state recognition unit 240 generates semantic information such as “The user is alone and troubled.”

When the user 10 is recognized, the state recognition unit 240 reads out information about the user 10. The information about the user 10 is stored in the storage unit 230 in association with a face image of the user 10. The information about the user 10 includes name, occupation, and age of the user 10, among others. If the user 10 is an artist, information about the user 10 may include information about past performances, such as works created by the user 10 in the past or videos in which the user 10 has appeared. For example, the state recognition unit 240 may estimate an occupation of the user 10 from a past speech content of the user 10.

The emotion determination unit 242 determines an emotion value indicating emotion of the user 10 based on information analyzed by the sensor module unit 220 and a state of the user 10 recognized by the state recognition unit 240. For example, information analyzed by the sensor module unit 220 and a recognized state of the user 10 are input to a pre-trained neural network to obtain an emotion value indicating emotion of the user 10.

Here, the emotion value indicating emotion of the user 10 is a value indicating the positivity or negativity of the user's emotion. For example, if the user's emotion is a bright emotion accompanied by pleasure or comfort, such as “joy,” “pleasure,” “comfort,” “relief,” “excitement,” “ease,” or “fulfillment,” the value is positive, and the brighter the emotion, the larger the value. If the user's emotion is an unpleasant emotion, such as “anger,” “sorrow,” “discomfort,” “anxiety,” “sadness,” “worry,” or “emptiness,” the value is negative, and the more unpleasant the emotion, the greater an absolute value of the negative value. If the user's emotion is none of the above (“normal”), the value is zero.

The emotion determination unit 242 also determines an emotion value indicating the emotion of the avatar 100 based on the information analyzed by the sensor module unit 220 and a state of the user 10 recognized by the state recognition unit 240.

Emotion values of the avatar 100 include emotion values for each of multiple emotion categories, for example, values (0 to 5) indicating an intensity of each of “joy,” “anger,” “sorrow,” and “pleasure.”

Specifically, the emotion determination unit 242 determines an emotion value indicating emotion of the avatar 100 according to a rule for updating an emotion value of the avatar 100, which is defined in association with information analyzed by the sensor module unit 220 and a state of the user 10 recognized by the state recognition unit 240.

For example, when the state recognition unit 240 recognizes that the user 10 seems lonely, the emotion determination unit 242 increases a “sorrow” emotion value of the avatar 100. When the state recognition unit 240 recognizes that the user 10 is smiling, the emotion determination unit 242 increases “joy” emotion value of the avatar 100.

The emotion determination unit 242 may further determine an emotion value indicating the emotion of the avatar 100 by also considering a state of the avatar 100. For example, when the user 10 does not speak to the avatar 100 or responds in a rough manner, the emotion determination unit 242 may increase a “sorrow” emotion value of the avatar 100. Furthermore, if the user 10 continues to respond roughly, the emotion determination unit 242 may increase an “anger” emotion value.

The behavior recognition unit 244 recognizes behavior of the user 10 based on the information analyzed by the sensor module unit 220 and a state of the user 10 recognized by the state recognition unit 240. For example, information analyzed by the sensor module unit 220 and a recognized state of the user 10 are input to a pre-trained neural network to obtain probabilities of each of a plurality of predefined behavior categories (for example, “laughing,” “angry,” “asking question,” “sad”), and the behavior category with the highest probability is recognized as behavior of the user 10.

As described above, in this embodiment, the control device 200 identifies the user 10 and acquires speech contents of the user 10, but when acquiring and using such speech content, necessary consent according to laws and regulations is obtained from the user 10, and a behavior control system of the avatar 100 according to this embodiment takes privacy and personal information protection of the user 10 into consideration.

The behavior determination unit 246 determines behavior of an avatar corresponding to behavior of the user 10 based on an emotion value of the user 10 and an emotion value of the avatar. Specifically, the behavior determination unit 246 determines the behavior corresponding to the behavior of the user 10 recognized by the behavior recognition unit 244 based on the current emotion value of the user 10 determined by the emotion determination unit 242, the history data 232 of past emotion values determined by the emotion determination unit 242 before the current emotion value of the user 10 is determined, and the emotion value of the avatar 100. In this embodiment, the behavior determination unit 246 uses the most recent emotion value included in the history data 232 as the past emotion value of the user 10, but the disclosed technology is not limited to this aspect. For example, the behavior determination unit 246 may use a plurality of most recent emotion values as the past emotion values of the user 10, or may use the emotion value from a unit period such as one day ago. The behavior determination unit 246 may also determine behavior corresponding to behavior of the user 10 by further considering history of past emotion values of the avatar 100 in addition to a current emotion value of the avatar 100. The behavior determined by the behavior determination unit 246 includes gestures performed by the avatar 100 or speech content of the avatar 100.

The behavior determination unit 246 according to this embodiment determines, as behavior corresponding to behavior of the user 10, behavior of the avatar 100 associated with the response rule 231 when a combination including at least one of a past emotion value and current emotion value of the user 10, the emotion value of the avatar 100, and the behavior of the user 10 satisfies the conditions of a preset response rule. For example, when a past emotion value of the user 10 is positive and a current emotion value is negative, the behavior determination unit 246 determines a behavior for changing the emotion value of the user 10 to a more positive value as behavior corresponding to behavior of the user 10.

The behavior determination unit 246 may also determine the behavior corresponding to behavior of the user 10 based on the emotion of the avatar 100. For example, when the avatar 100 is verbally abused by the user 10 or treated rudely by the user 10 (that is, when the user's reaction is poor), when ambient noise is loud and voice of the user 10 cannot be detected, or when a “anger” or “sorrow” emotion value of the avatar 100 increases, the behavior determination unit 246 may determine a behavior corresponding to the increase in the “anger” or “sorrow” emotion value as behavior corresponding to behavior of the user 10. Conversely, when the user's reaction is good or a “joy” or “pleasure” emotion value of the avatar 100 increases, the behavior determination unit 246 may determine a behavior corresponding to the increase in the “joy” or “pleasure” emotion value as behavior corresponding to behavior of the user 10. The behavior determination unit 246 may also determine behavior for the user 10 whose “anger” or “sorrow” emotion value of the avatar 100 has increased that is different from behavior for the user 10 whose “joy” or “pleasure” emotion value of the avatar 100 has increased. In this way, the behavior determination unit 246 may determine different behaviors according to emotion of the avatar 100 itself or how the user 10's behavior has changed emotion of the avatar 100.

The response rule 231 defines the behavior of the avatar 100 according to the combination of the past and current emotion values of the user 10, the emotion value of the avatar 100, and the behavior of the user 10. For example, when the past emotion value of the user 10 is positive, the current emotion value is negative, and the behavior of the user 10 is “sad,” the response rule 231 specifies a combination of gestures and speech content for the avatar 100 to encourage the user 10 with gestures and questions.

For example, the response rule 231 defines the behavior of the avatar 100 for all combinations of emotion value patterns of the avatar 100 (1296 patterns, with each of “joy,” “anger,” “sorrow,” and “pleasure” having values from 0 to 5, i.e., 6 values to the 4th power), combination patterns of past and current emotion values of the user 10, and behavior patterns of the user 10. That is, for each pattern of the avatar 100's emotion values, for each combination of past and current emotion values of the user 10 (such as negative and negative, negative and positive, positive and negative, positive and positive, negative and normal, and normal and normal), behavior of the avatar 100 according to the behavior pattern of the user 10 is defined. The behavior determination unit 246 may also determine behavior of the avatar 100 using the history data 232 when the user 10 makes a statement indicating an intention to continue a conversation from a previous topic, such as “I want to talk about the topic we discussed last time.”

The response rule 231 may define, for each of the 1296 patterns of the avatar 100's emotion values, at most one of gesture and speech content as the behavior of the avatar 100. Alternatively, the response rule 231 may define, for each group of patterns of the avatar 100's emotion values, at least one of gesture and speech content as behavior of the avatar 100.

Each gesture included in the behavior of the avatar 100 defined in the response rule 231 has a corresponding predetermined intensity. Each speech content included in behavior of the avatar 100 defined in the response rule 231 has a corresponding predetermined intensity.

For example, the response rule 231 may define the behavior of the avatar 100 according to the user 10 being an artist. For example, the response rule 231 may define actions related to the performance of the user 10.

For example, the behavior determination unit 246 determines actions related to the creativity of the user 10 as an artist. For example, the behavior determination unit 246 determines actions that draw out inspiration and creativity in the user 10. The behavior determination unit 246 may also determine actions to improve management of projects involving the user 10.

The behavior determination unit 246 may also determine actions related to the expressiveness of the user 10 as an artist. For example, the behavior determination unit 246 determines actions to improve self-expression of the user 10. The behavior determination unit 246 may also determine actions related to emotion management for the user 10. The behavior determination unit 246 may determine actions related to mental training for the user 10. Specifically, the behavior determination unit 246 determines actions to propose methods for relieving tension in the user 10 or to propose communication techniques with the audience.

The memory control unit 248 determines whether to store data including behavior of the user 10 in the history data 232 based on a predetermined intensity of behavior determined by the behavior determination unit 246 and an emotion value of the avatar 100 determined by the emotion determination unit 242.

Specifically, when the total value of the intensities, which is the sum of the total of emotion values for each of multiple emotion categories of the avatar 100, the predetermined intensity for gestures included in behavior determined by the behavior determination unit 246, and a predetermined intensity for speech content included in behavior determined by the behavior determination unit 246, is equal to or greater than a threshold, it is determined that data including the behavior of the user 10 is to be stored in the history data 232.

When the memory control unit 248 determines to store data including behavior of the user 10 in the history data 232, it stores the behavior determined by the behavior determination unit 246, information analyzed by the sensor module unit 220 from the present time to a certain period in the past (for example, all kinds of surrounding information such as audio, images, odors, etc.), and a state of the user 10 recognized by the state recognition unit 240 (for example, the facial expression, emotion, etc. of the user 10) in the history data 232.

The behavior control unit 250 controls the avatar 100 and the controlled object 252 based on the behavior of the avatar 100 determined by the behavior determination unit 246. Specifically, the behavior control unit 250 controls movement of the avatar 100 and operation of the controlled object 252 related to movement of the avatar 100 based on behavior of the avatar 100 associated with the response rule 231 determined by the behavior determination unit 246. For example, in a case where the avatar 100 and the user 10 are having a conversation, if the behavior determination unit 246 determines a behavior including speech, the behavior control unit 250 controls the avatar 100 to take a predetermined action and causes the speaker 2522 included in the controlled object 252 to output voice of the avatar 100. At this time, the behavior control unit 250 may determine a speech speed based on an emotion value of the avatar 100. For example, the behavior control unit 250 determines a faster speech speed as the emotion value of the avatar 100 increases. In this way, the behavior control unit 250 determines an execution mode of the behavior determined by the behavior determination unit 246 based on the emotion value determined by the emotion determination unit 242.

The behavior control unit 250 may recognize changes in emotion of the user 10 in response to execution of behavior determined by the behavior determination unit 246. For example, changes in emotion may be recognized based on voice or facial expression of the user 10. In addition, changes in emotion may be recognized based on detection of an impact by a touch sensor included in the sensor unit 210. If an impact is detected by the touch sensor included in the sensor unit 210, it may be recognized that emotion of the user 10 has worsened, or if detection result of the touch sensor included in the sensor unit 210 indicates that the user 10 is laughing or happy, it may be recognized that emotion of the user 10 has improved. Information indicating reaction of the user 10 is output to the communication processing unit 280.

After the behavior control unit 250 executes the behavior determined by the behavior determination unit 246 in an execution mode determined according to emotion of the avatar 100, the emotion determination unit 242 further changes an emotion value of the avatar 100 based on the user's reaction to the executed behavior. Specifically, if a user's reaction to behavior with respect to the user is not poor, which is determined by the behavior determination unit 246 and further is executed in a manner determined by the behavior control unit 250, the emotion determination unit 242 increases a “joy” emotion value of the avatar 100. If a user's reaction to behavior with respect to the user is poor, which is determined by the behavior determination unit 246 and further is executed in a manner determined by the behavior control unit 250, the emotion determination unit 242 increases the “sorrow” emotion value of the avatar 100.

Furthermore, the behavior control unit 250 expresses emotion of the avatar 100 based on a determined emotion value of the avatar 100. For example, when a “joy” emotion value of the avatar 100 is increased, the behavior control unit 250 controls movement of the avatar 100 to make a joyful gesture. When a “sorrow” emotion value of the avatar 100 is increased, the behavior control unit 250 controls movement of the avatar 100 to make the avatar 100 take a slumped posture.

For example, when the user 10 says “I was nervous” and is smiling, a “joy” emotion value of the avatar 100 increases, and the behavior control unit 250 makes a statement such as “You performed well.” When the user 10 says “I was nervous” and is sad, a “sorrow” emotion value of the avatar 100 increases, and the behavior control unit 250 makes statements such as “You can do it, Mr./Ms. A” or “Let's try BBB before the next performance.”

In this way, for example, the behavior control unit 250 may execute actions related to emotion management for the user 10 or actions proposing methods to relieve tension for the user 10. The behavior control unit 250 may also execute actions proposing communication techniques with the audience. The behavior control unit 250 may also execute actions to improve self-expression for the user 10. The behavior control unit 250 may also execute actions related to mental training for the user 10. That is, the behavior control unit 250 may execute actions related to expressiveness for the user 10.

The behavior control unit 250 may also execute actions related to creativity for the user 10. The behavior control unit 250 may execute actions to draw out inspiration and creativity in the user 10. The behavior control unit 250 may also execute actions to improve the management of projects involving the user 10.

The communication processing unit 280 is responsible for communication with the server 300. As described above, the communication processing unit 280 transmits user reaction information to the server 300. The communication processing unit 280 also receives updated response rules from the server 300. When the communication processing unit 280 receives updated response rules from the server 300, it updates the response rules 231.

The output unit 291 implements the above-described output functions. Details of the output unit 291 will be described later.

The server 300 communicates with the control devices 200 to 202 that control the avatars 100 to 102, receives user reaction information transmitted from the control devices 200 to 202, and updates the response rules based on response rules that include actions for which positive reactions were obtained.

### (Behavior Determination Based on Character)

So far, the case where the behavior determination unit 246 determines behaviors of the avatar 100 based on states recognized by the state recognition unit 240 has been described. On the other hand, the behavior determination unit 246 may determine behaviors of the avatar 100 not only based on the user's states but also based on set characters. In this case, the behavior determination unit 246 may acquire an age (target age) associated with character from the character data 233 and determine a behavior of the avatar 100 based on the acquired target age.

That is, the behavior determination unit 246 determines a behavior of the avatar 100 based on a state recognized by the state recognition unit 240 and a set character or an age associated with the character. This makes it possible to have the avatar 100 perform appropriate actions according to the user's age. In particular, it becomes possible to restrict actions by the avatar 100 that are not suitable for young users (for example, outputting violent content).

In system 5, characters are set in advance. The character setting is input as a prompt (namely, instruction sentence). The prompt may be input via an input device provided in the control device 200 or via an external device such as a server communicably connected to the control device 200. In the prompt, a name of the character may be specified, or an ID assigned to each character may be specified.

For example, the behavior determination unit 246 may determine an action to output information to an output device such as the display device 2521 or the speaker 2522 provided in the control device 200, in a manner according to a target age. For example, the behavior determination unit 246 may change voice of the avatar 100 output from the speaker 2522 to character's voice.

For example, the behavior determination unit 246 may determine an action to output voice or messages using text composed of words appropriate for a target age. Here, the words that can be used for each age are preset. The behavior determination unit 246 acquires a target age from the character data 233.

For example, suppose that words “What's wrong?” and “How may I help you?” are stored in the storage unit 230 as words to be output when the avatar 100 performs an action of “speaking to” the user 10. “What's wrong?” is associated with an age “under 12,” and “How may I help you?” is associated with an age “12 and above.” For example, the behavior determination unit 246 determines to output the phrase “How may I help you?” when a target age is “18 and above.” The behavior determination unit 246 determines to output the phrase “What's wrong?” when a target age is “3 to 7 years old.”

In this way, the control device 200 can restrict actions unsuitable for young users and improve familiarity for young users by changing a voice tone and words output according to a target age.

Furthermore, the behavior determination unit 246 determines an action to output content corresponding to a character, to an output device (such as display device 2521). For example, the behavior determination unit 246 determines an action to display video content (movies, animations, etc.) featuring a character on the display device 2521.

The behavior determination unit 246 may also determine an action to output educational content according to a target age. Here, educational content includes text, video, audio, etc., related to learning subjects such as English, mathematics, Japanese, science, and social studies. The educational content may also be interactive content in which the user inputs answers to questions. For example, the behavior determination unit 246 determines an action to display calculation problems corresponding to a grade for a target age on the display device 2521. For example, the behavior determination unit 246 determines to display addition problems when a target age is “under 8 years old” and multiplication problems when a target age is “8 years old and above.”

The behavior determination unit 246 may also determine an action to output content according to a target age, rather than a character, to an output device. In this case, the content may be content featuring a character or content not dependent on a character, such as generally known folktales or fairy tales.

The content corresponding to the character, and a target age and an educational content according to a grade may be stored in advance in the storage unit 230 or may be acquired from an external device such as a server communicably connected to the control device 200.

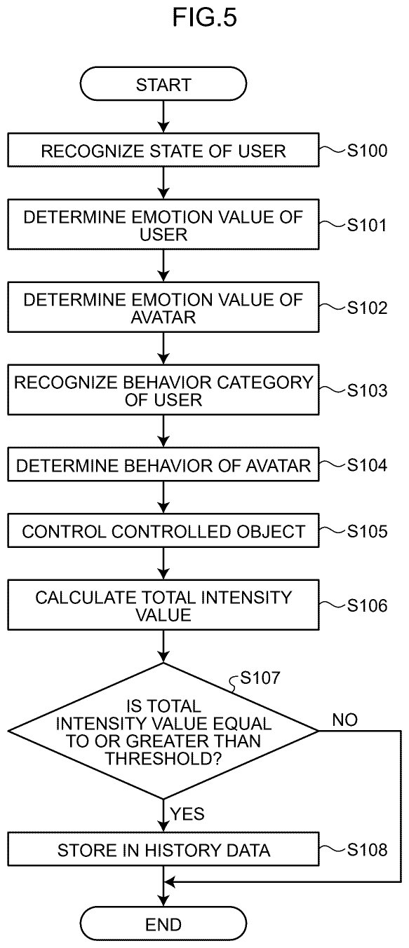

FIG. 4 is a diagram schematically illustrating an example of an operation flow related to character setting. Note that “S” in the operation flow represents steps to be executed.

In step S50, the control device 200 accepts a character setting. Then, in step S51, the control device 200 outputs a screen corresponding to a character of the avatar 100 (for example, screen displaying appearance of character).

In step S52, the behavior determination unit 246 acquires a target age corresponding to a set character from the character data 233.

FIG. 5 is a diagram schematically illustrating an example of an operation flow related to determining a behavior of the avatar 100. The operation flow shown in FIG. 5 is repeatedly executed. At this time, it is assumed that information analyzed by the sensor module unit 220 is being input. Note that “S” in the operation flow represents steps to be executed.

First, in step S100, the state recognition unit 240 recognizes a state of the user 10 based on information analyzed by the sensor module unit 220. When the user 10 is recognized, the state recognition unit 240 reads out information about the user 10.

In step S101, the emotion determination unit 242 determines a emotion value indicating emotion of the user 10 based on information analyzed by the sensor module unit 220 and a state of the user 10 recognized by the state recognition unit 240.

In step S102, the emotion determination unit 242 determines an emotion value indicating emotion of the avatar 100 based on information analyzed by the sensor module unit 220 and a state of the user 10 recognized by the state recognition unit 240. The emotion determination unit 242 adds the determined emotion value of the user 10 to the history data 232.

In step S103, the behavior recognition unit 244 recognizes a behavior category of the user 10 based on information analyzed by the sensor module unit 220 and a state of the user 10 recognized by the state recognition unit 240.

In step S104, the behavior determination unit 246 determines a behavior of the avatar 100 based on a combination of a current emotion value of the user 10 determined in step S101 and a past emotion value included in the history data 232, an emotion value of the avatar 100, a behavior of the user 10 recognized by the behavior recognition unit 244, and the response rule 231.

For example, when the user 10 says “I was nervous” and is smiling, a “joy” emotion value of the avatar 100 increases, and the behavior determination unit 246 determines a behavior of the avatar 100 to make a statement such as “You performed well.”

In step S105, the behavior control unit 250 controls the controlled object 252 based on a behavior determined by the behavior determination unit 246.

In step S106, the memory control unit 248 calculates a total value of intensities based on a predetermined intensity of a behavior determined by the behavior determination unit 246 and an emotion value of the avatar 100 determined by the emotion determination unit 242.

In step S107, the memory control unit 248 determines whether a total value of intensities is equal to or greater than a threshold. If the total value of the intensities is less than the threshold, the process ends without storing data including the behavior of the user 10 in the history data 232. On the other hand, if the total value of the intensities is equal to or greater than the threshold, the process proceeds to step S108.

In step S108, the memory control unit 248 stores a behavior determined by the behavior determination unit 246, information analyzed by the sensor module unit 220 from the present time to a certain period in the past, and a state of the user 10 recognized by the state recognition unit 240; in the history data 232.

The control device 200 recognizes actions of an artist and determines actions of an avatar corresponding to the recognized actions of the artist. Then, the control device 200 controls the avatar based on the determined actions of the avatar. As a result, the control device 200 can have the avatar 100 perform appropriate actions for the artist. For example, the control device 200 can improve performance of the user 10.

The control device 200 determines actions related to creativity of the user 10. As a result, the control device 200 can draw out inspiration and creativity in the user 10. For example, the control device 200 can support the creation of works by the user 10. The control device 200 can also provide advice to improve the management of projects involving the user 10.

The control device 200 also determines actions related to expressiveness of the user 10. As a result, the control device 200 can improve self-expression of the user 10. The control device 200 can also improve emotion management for the user 10. For example, the control device 200 can relieve tension in the user 10. The control device 200 can also improve communication techniques with the audience.

As described above, according to the control device 200, an emotion value indicating emotion of the avatar 100 is determined based on a user's state, and it is determined whether to store data including the behavior of the user 10 in the history data 232 based on the emotion value of the avatar 100. As a result, it is possible to suppress capacity of the history data 232 that stores data including the behavior of the user 10. For example, when the control device 200 determines that a user's state 10 years later is the same as that 10 years ago, the control device 200 can present all kinds of surrounding information at that time, such as a state of the user 10 (for example, facial expression, emotion, etc. of user 10), as well as audio, images, odors, etc., to the user 10 via the avatar 100, the display device 2521, the speaker 2522, etc., by reading the history data 232 from 10 years ago.

According to the control device 200, it is also possible to have the avatar 100 perform appropriate actions in response to a behavior of the user 10. Conventionally, the user's behavior was classified and a behavior of an avatar, including facial expressions and appearance, was determined. In contrast, the control device 200 determines a current emotion value of the user 10 and performs actions for the user 10 based on past and current emotion values. Therefore, for example, if the user 10 was cheerful yesterday but is feeling down today, the control device 200 can have the avatar 100 say something like “You were cheerful yesterday, but what's wrong today?” The control device 200 can also have the avatar 100 make statements with gestures. For example, if the user 10 was feeling down yesterday but is cheerful today, the control device 200 can have the avatar 100 say something like “You were feeling down yesterday, but you look cheerful today.” If the user 10 was cheerful yesterday and is even more cheerful today, the control device 200 can have the avatar 100 say something like “You're even more cheerful today than yesterday. Did something good happen?” In addition, for the user 10 whose emotion value is zero or higher and whose emotion value fluctuation remains within a certain range for a continued period, the control device 200 can have the avatar 100 say something like “You've been feeling stable and good recently.”

For example, the control device 200 can have the avatar 100 ask the user 10, “Did you finish the homework you mentioned yesterday?” and, if the user 10 answers “Yes, I did,” the avatar 100 can make a positive statement such as “Good job!” and perform a positive gesture such as clapping or a thumbs-up. For example, if the user 10 says, “The presentation I talked about the day before yesterday went well,” the control device 200 can have the avatar 100 make a positive statement such as “You did great!” and perform the above positive gesture. In this way, by having the avatar 100 controlled by the control device 200 act based on history of the user 10's state, it is expected that the user 10 will feel a sense of closeness to the avatar 100.

For example, a control system 1 can draw out inspiration and creativity in people engaged in creative activities and support the creation of works by controlling behaviors of the avatar 100 based on user's emotions. The control system 1 can also provide advice on project management and improvement of self-expression for the user 10.

For example, when the avatar 100 is an artist, the control system 1 controls behaviors of the avatar 100 to draw out inspiration and creativity in the user 10 and support creation of works as described above. Specifically, the control system 1 has the avatar 100 to reproduce a painter who paints in the style that the user 10 desires. For example, the control system 1 reproduces famous painters, ordinary people, art teachers, etc., as the avatar 100, and supports the user 10 in drawing paintings in each style so as to draw out inspiration in the user 10.

In the above embodiment, the case where the control device 200 recognizes the user 10 using the face image of the user 10 has been described, but the disclosed technology is not limited to this aspect. For example, the control device 200 may recognize the user 10 using voice of the user 10, an email address of the user 10, an SNS ID of the user 10, an ID card with a built-in wireless IC tag possessed by the user 10, or the like.

Note that the control device 200 is an example of an information processing device for controlling the avatar 100. The application of behavior control by the control device 200 is not limited to the avatar 100 and can be applied to various electronic devices. The functions of the server 300 may be implemented by one or more computers. At least a part of functions of the server 300 may be implemented by a virtual machine. At least a part of functions of the server 300 may be implemented in the cloud.

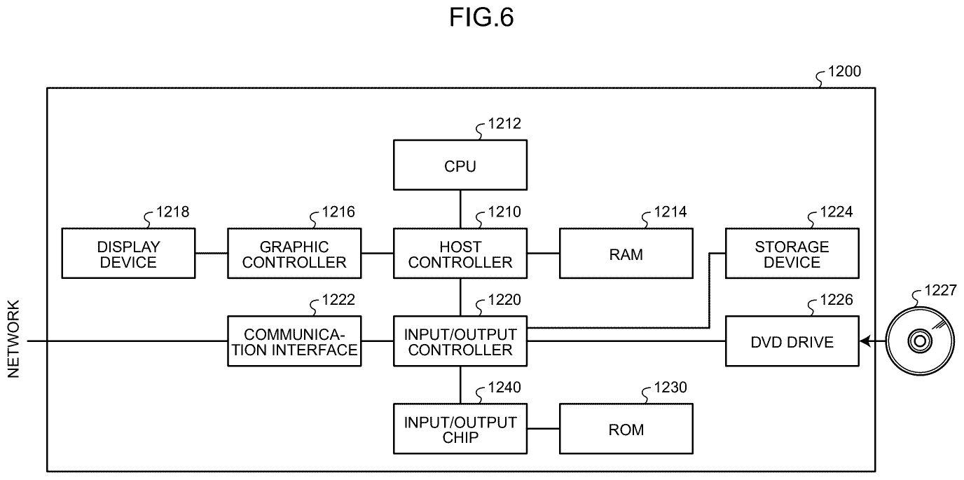

FIG. 6 is a diagram schematically illustrating an example of a hardware configuration of a computer 1200 functioning as the control device 200 and the server 300. A program installed in the computer 1200 can cause the computer 1200 to function as one or more “units” of the device according to the present embodiment, or to execute operations or the one or more “units” associated with the device according to the present embodiment, and/or to execute processes or steps of the processes according to the present embodiment. Such a program may be executed by a CPU 1212 to perform specific operations associated with some or all of the blocks in the flowcharts and block diagrams described in this specification.

The computer 1200 according to this embodiment includes the CPU 1212, a RAM 1214, and a graphic controller 1216, which are interconnected by a host controller 1210. The computer 1200 also includes a communication interface 1222, a storage device 1224, a DVD drive 1226, and input/output units such as an IC card drive, which are connected to the host controller 1210 via an input/output controller 1220. The DVD drive 1226 may be a DVD-ROM drive or a DVD-RAM drive among others. The storage device 1224 may be a hard disk drive or a solid-state drive among others. The computer 1200 also includes a ROM 1230 and legacy input/output units such as a keyboard, which are connected to the input/output controller 1220 via an input/output chip 1240.

The CPU 1212 operates according to programs stored in the ROM 1230 and the RAM 1214, thereby controlling each unit. The graphic controller 1216 acquires image data generated by the CPU 1212 in a frame buffer provided in the RAM 1214 or in itself, and causes the display device 1218 to display the image data.

The communication interface 1222 communicates with other electronic devices via a network. The storage device 1224 stores programs and data used by the CPU 1212 in the computer 1200. The DVD drive 1226 reads programs or data from a DVD-ROM 1227 or the like and provides them to the storage device 1224. The IC card drive reads programs and data from an IC card and/or writes programs and data to an IC card.

The ROM 1230 stores programs such as a boot program executed by the computer 1200 at activation and/or programs dependent on hardware of the computer 1200. The input/output chip 1240 may also connect various input/output units to the input/output controller 1220 via USB ports, parallel ports, serial ports, keyboard ports, mouse ports, and the like.

Programs are provided by a computer-readable storage media such as the DVD-ROM 1227 or IC cards. The programs are read from the computer-readable storage media and installed in the storage device 1224, the RAM 1214, or the ROM 1230, which are also examples of the computer-readable storage media, and executed by the CPU 1212. The information processing described in these programs is read by the computer 1200 and enables cooperation between the program and various types of hardware resources. The device or method may be configured by realizing information operations or processing using the computer 1200.

For example, when communication is performed between the computer 1200 and an external device, the CPU 1212 executes a communication program loaded into the RAM 1214 and instructs the communication interface 1222 to perform communication processing based on processing described in the communication program. Under the control of the CPU 1212, the communication interface 1222 reads transmission data stored in a transmission buffer area provided in the RAM 1214, the storage device 1224, the DVD-ROM 1227, or a recording medium such as an IC card, and transmits the read transmission data to a network, or writes the received data received from a network to a reception buffer area provided on the recording medium.

The CPU 1212 may cause all or necessary parts of files or databases stored in an external recording media such as the storage device 1224, the DVD drive 1226 (namely, DVD-ROM 1227), or an IC card to be read into the RAM 1214, and perform various types of processing on data in the RAM 1214. The CPU 1212 may then write back the processed data to the external recording media.

Various types of information such as programs, data, tables, and databases may be stored in a recording media and subjected to information processing. The CPU 1212 may perform various types of processing on data read from the RAM 1214, including various types of operations, information processing, conditional judgments, conditional branches, unconditional branches, information search/replacement, etc., as described throughout this disclosure and specified by an instruction sequence of the program, and write back the results to the RAM 1214. The CPU 1212 may also search for information in files, databases, etc., in the recording media. For example, when a plurality of entries having attribute values of a first attribute associated with attribute values of a second attribute are stored in the recording media, the CPU 1212 may search for entries matching a specified condition of the first attribute among the plurality of entries, read an attribute value of the second attribute stored in the entry, and thereby acquire an attribute value of the second attribute associated with the first attribute that satisfies a predetermined condition.

The above-described programs or software modules may be stored on a computer-readable storage medium on or near the computer 1200. A recording medium such as a hard disk or a RAM provided in a server system connected to a dedicated communication network or the Internet can be used as a computer-readable storage medium, thereby providing a program to the computer 1200 via the network.

Blocks in flowcharts and block diagrams in this embodiment may represent steps of a process in which operations are executed or “units” of a device that perform the operations. Specific steps and “units” may be implemented by dedicated circuits, programmable circuits supplied with computer-readable instructions stored on a computer-readable storage medium, and/or processors supplied with computer-readable instructions stored on a computer-readable storage medium. The dedicated circuits may include digital and/or analog hardware circuits, and include integrated circuits (ICs) and/or discrete circuits. The programmable circuits may include, for example, reconfigurable hardware circuits such as field-programmable gate arrays (FPGAs) and programmable logic arrays (PLAs), which include logical AND, logical OR, exclusive OR, NAND, NOR, and other logic operations, flip-flops, registers, and memory elements.

A computer-readable storage medium may include any tangible device capable of storing instructions executed by an appropriate device, and as a result, a computer-readable storage medium having instructions stored therein constitutes a product including instructions that can be executed to perform operations specified in flowcharts or block diagrams. Examples of the computer-readable storage media may include electronic storage media, magnetic storage media, optical storage media, electromagnetic storage media, semiconductor storage media, and the like. More specific examples of computer-readable storage media may include floppy disks, diskettes, hard disks, random access memory (RAM), read-only memory (ROM), erasable programmable read-only memory (EPROM or flash memory), electrically erasable programmable read-only memory (EEPROM), static random access memory (SRAM), compact disc read-only memory (CD-ROM), digital versatile discs (DVD), Blu-ray (Registered trademark) discs, memory sticks, integrated circuit cards, and the like.

Computer-readable instructions may include assembler instructions, instruction set architecture (ISA) instructions, machine instructions, machine-dependent instructions, microcode, firmware instructions, state-setting data, or source code or object code written in any combination of one or more programming languages, including object-oriented programming languages such as Smalltalk, JAVA (Registered trademark), C++, and conventional procedural programming languages such as the “C” programming language or similar programming languages.

Computer-readable instructions may be provided locally or via a local area network (LAN), wide area network (WAN) such as the Internet, to a general-purpose computer, special-purpose computer, or other programmable data processing device processor or programmable circuit, to generate means for executing operations specified in flowcharts or block diagrams. Examples of processors include computer processors, processing units, microprocessors, digital signal processors, controllers, microcontrollers, and the like.

### OTHER EMBODIMENTS

The above-described control device 200 may be mounted in a stuffed animal or robot, or may be applied to a control device wirelessly or wiredly connected to a controlled object device (such as a speaker or camera) mounted in a stuffed animal or robot.

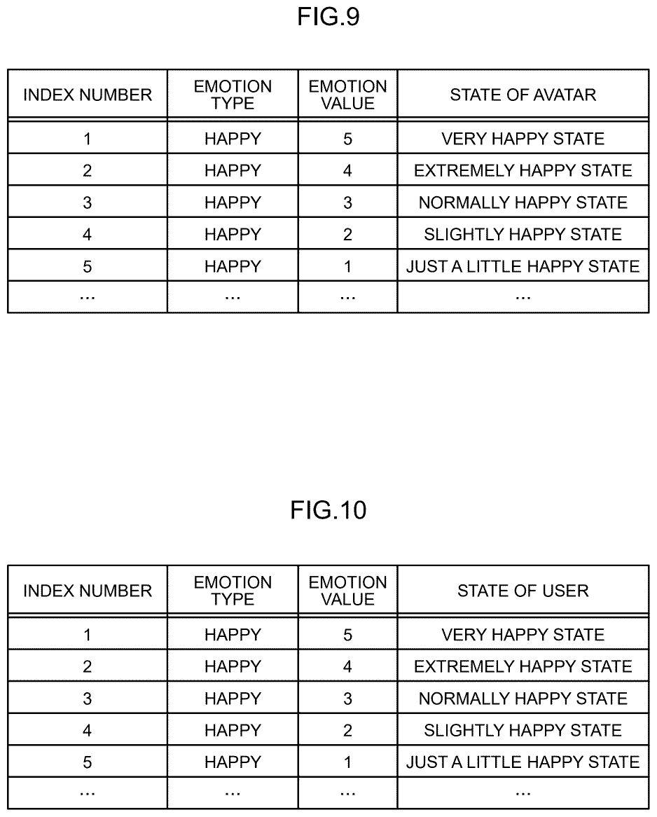

The emotion determination unit 242 may determine user's emotion according to a specific mapping. Specifically, the emotion determination unit 242 may determine user's emotion according to an emotion map, which is a specific mapping (see FIG. 9).

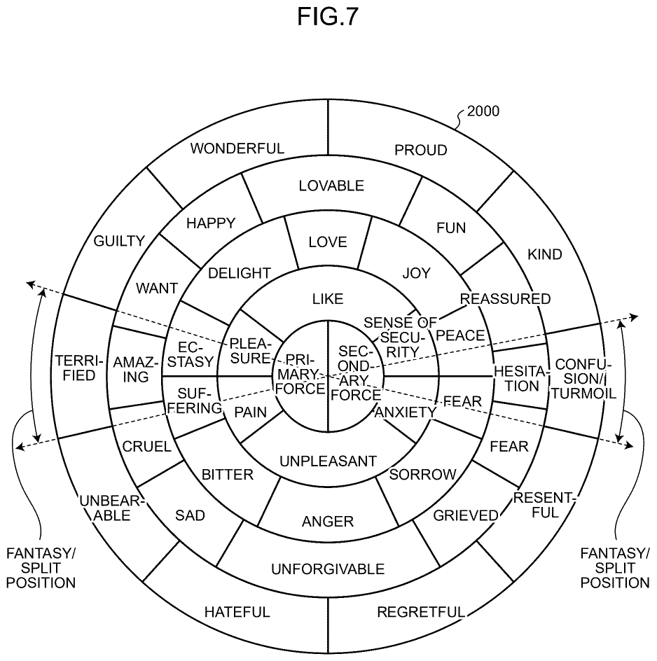

FIG. 7 is a diagram illustrating an emotion map 2000 in which multiple emotions are mapped. In the emotion map 2000, emotions are arranged concentrically in a radial pattern from the center. The closer to the center of the concentric circles, the more primitive emotions are arranged. On outer circles, emotions representing states or behaviors arising from mental states are arranged. Emotions are a concept that also includes affect and mental states. On a left side of the concentric circles, emotions generally generated from reactions occurring in brain are arranged. On a right side of the concentric circles, emotions generally induced by situational judgment are arranged. In upward and downward directions of the concentric circles, emotions that are both generated from reactions in brain and induced by situational judgment are arranged. In addition, “comfort” emotions are arranged on an upper side of the concentric circles, and “discomfort” emotions are arranged on a lower side. In this way, in the emotion map 2000, multiple emotions are mapped based on a structure in which emotions are generated, and emotions that are likely to occur simultaneously are mapped close to each other.

(1) For example, when an emotion engine, which is the emotion determination unit 242 for controlling emotions of the avatar 100, detects emotions at about 100 msec intervals, a timing for determining a reactive behavior (such as backchanneling) of the avatar 100 may be set to the same timing as the detection frequency (100 msec) of the emotion engine, even if the frequency is low, or may be set to an earlier timing. The detection frequency of the emotion engine may be interpreted as a sampling rate.

By detecting emotions at about 100 msec intervals and immediately performing reactive behaviors (such as backchanneling) in response, unnatural backchanneling is avoided and natural, context-aware dialogue can be achieved. The avatar 100 performs reactive behaviors (such as backchanneling) according to the direction and degree (intensity) in the mandala of the emotion map 2000. The detection frequency (sampling rate) of the emotion engine is not limited to 100 msec and may be changed according to a situation (such as during sports) or the user's age.

(2) The direction and degree of emotions are preset with reference to the emotion map 2000, and movement and intensity of backchanneling may be set accordingly. For example, when the avatar 100 feels stability or relief, the avatar 100 continues to listen while nodding. When the avatar 100 feels anxiety, hesitation, or suspicion, the avatar 100 may tilt its head or stop shaking its head.

These emotions are distributed in the 3 o'clock direction of the emotion map 2000, and usually move back and forth between relief and anxiety. In a right half of the emotion map 2000, situational awareness takes precedence over internal sensations, resulting in a calm impression.

(3) When the avatar 100 feels pleasure from being praised, a filler such as “Ah-” may be inserted before the line, and when the avatar 100 feels pain from harsh words, a filler such as “Ugh!” may be inserted before the line. Avatar 100 may also include physical reactions such as crouching while saying “Ugh!” These emotions are distributed around the 9 o'clock direction of the emotion map 2000.

(4) In a left half of the emotion map 2000, internal sensations (reactions) take precedence over situational awareness. Therefore, it may give impression of an involuntary reaction.

When the avatar 100 feels an internal sensation (reaction) of conviction and also feels a favorable impression in situational awareness, the avatar 100 may nod deeply while looking at the other person and say “Mm-hmm.” In this way, the avatar 100 may generate balanced favorable actions toward the other person, such as acceptance or tolerance. Such emotions are distributed around the 12 o'clock direction of the emotion map 2000.

Conversely, when the avatar 100 feels an internal sensation (reaction) of discomfort and also feels a negative impression in situational awareness, the avatar 100 may shake its head when feeling disgust, or glare at the other person when feeling hatred. Such emotions are distributed around the 6 o'clock direction of the emotion map 2000.

(5) The inside of the emotion map 2000 represents mind, and the outside represents behavior, so the farther from the center, the more visible (behaviorally expressed) emotions become.

(6) When listening to someone while feeling relief, which is distributed around the 3 o'clock direction of the emotion map 2000, the avatar 100 may lightly nod and say “Hmm-hmm,” but when it comes to love around the 12 o'clock direction, the avatar 100 may nod more strongly with a deep nod.

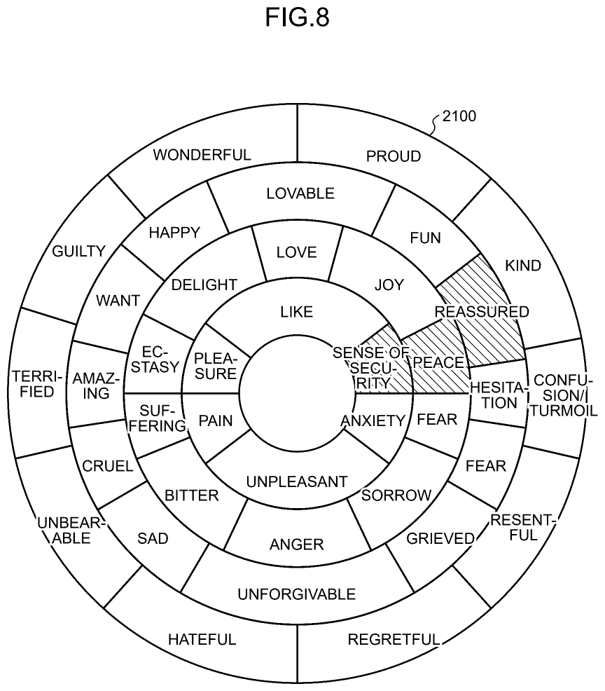

The emotion determination unit 242 inputs information analyzed by the sensor module unit 220 and a recognized state of the user 10 into a pre-trained neural network, obtains emotion values indicating each emotion shown in the emotion map 2000, and determines emotion of the user 10. This neural network is pre-trained based on multiple training data combining information analyzed by the sensor module unit 220 and a recognized state of the user 10 with emotion values indicating each emotion shown in the emotion map 2000. FIG. 8 is a diagram illustrating another example of the emotion map. In this neural network, as in the emotion map 2100 shown in FIG. 8, emotions placed close to each other are learned to have similar values. FIG. 8 shows an example in which multiple emotions such as “relief,” “peace,” and “reassurance” have similar emotion values.

The emotion determination unit 242 may also determine emotion of the avatar 100 according to a specific mapping. Specifically, the emotion determination unit 242 inputs information analyzed by the sensor module unit 220, a state of the user 10 recognized by the state recognition unit 240, and a state of the avatar 100 into a pre-trained neural network, obtains emotion values indicating each emotion shown in the emotion map 2000, and determines emotion of the avatar 100. This neural network is pre-trained based on multiple training data combining information analyzed by the sensor module unit 220, a recognized state of the user 10, and a state of the avatar 100 with emotion values indicating each emotion shown in the emotion map 2000. For example, the neural network is trained based on training data indicating that when the avatar 100 is recognized as being petted by the user 10 from an output of a touch sensor, an emotion value for “happy” is 3, or when the avatar 100 is recognized as being hit by the user 10 from an output of an acceleration sensor, an emotion value for “anger” is 3. In this neural network, as in the emotion map 2100 shown in FIG. 8, emotions placed close to each other are learned to have similar values.

The emotion determination unit 242 may also determine emotion of the avatar 100 based on a behavior content of the avatar 100 generated by a text generation model. Specifically, the emotion determination unit 242 inputs the behavior content of the avatar 100 generated by the text generation model into a pre-trained neural network, obtains emotion values indicating each emotion shown in the emotion map 2000, and integrates the obtained emotion values with the current emotion values of the avatar 100 to update the emotion of the avatar 100. For example, obtained emotion values and current emotion values of the avatar 100 may be averaged and integrated. This neural network is pre-trained based on multiple training data combining the text representing a behavior content of the avatar 100 generated by a text generation model with emotion values indicating each emotion shown in the emotion map 2000.

For example, when a behavior content of the avatar 100 generated by a text generation model is a speech content “That's great. You were lucky,” inputting this text into the neural network yields a high value for the emotion “happy,” and emotion of the avatar 100 is updated so that a emotion value for “happy” increases.

The behavior determination unit 246 generates a behavior content of the avatar by adding a fixed sentence for asking about a behavior content of the avatar corresponding to a user's behavior to a text representing the user's behavior, the user's emotion, and the avatar's emotion, and inputting it to the text generation model with dialogue functionality.

For example, the behavior determination unit 246 obtains text representing a state of the avatar 100 using an emotion table as shown in FIG. 9, based on the emotion of the avatar 100 determined by the emotion determination unit 242. FIG. 9 is a diagram illustrating an example of an emotion table. In the emotion table, each emotion type is assigned an index number for each emotion value, and text representing the state of the avatar 100 is stored for each index number.

When the emotion of the avatar 100 determined by the emotion determination unit 242 corresponds to index number “2,” the text “very happy state” is obtained. If emotion of the avatar 100 corresponds to multiple index numbers, multiple texts representing a state of the avatar 100 are obtained.

An emotion table as shown in FIG. 10 is also prepared for emotions of the user 10. FIG. 10 is a diagram illustrating an example of an emotion table. Here, when the user's behavior is speaking “AAA,” emotion of the avatar 100 is index number “2,” and emotion of the user 10 is index number “3,” a text “The avatar is in a very happy state. The user is in a normally happy state. The user said ‘AAA.’ How should the avatar respond?” is input to a text generation model to obtain a behavior content of the avatar. The behavior determination unit 246 determines a behavior of the avatar from this behavior content.

In this way, the avatar 100 can change its behavior according to the index number corresponding to its emotion, so the user is encouraged to feel as if the avatar 100 has a mind and to take actions such as talking to the avatar.

The behavior determination unit 246 may also generate a behavior content of the avatar by adding not only a text representing a user's behavior, user's emotion, and avatar's emotion, but also a text representing a content of the history data 232, and a fixed sentence for asking about a behavior content of the avatar corresponding to the user's behavior, and inputting it to dialogue function. This allows the avatar 100 to change its behavior according to history data representing the user's emotions and behaviors, so the user is encouraged to feel as if the avatar has a personality and to take actions such as talking to the avatar. The history data may also include emotions and behaviors of the avatar.

### Example 2 of Embodiment

Next, Example 2 of the embodiment will be described. Here, description focuses on differences from Example 1, and descriptions of the same configuration and processing as in Example 1 are omitted. An example of the system 5 according to this embodiment is schematically illustrated in FIG. 1.

Each control device is equipped with a learning support function that detects a user's learning status in various learning fields via each avatar and outputs information corresponding to the detected learning status. For example, each control device detects a user's level of understanding in a specific learning field and, by outputting learning guidance, learning plans, and learning resources (such as reference books) via the avatar according to a level of understanding, can output appropriate information to the user 10 according to a learning status of the user 10.

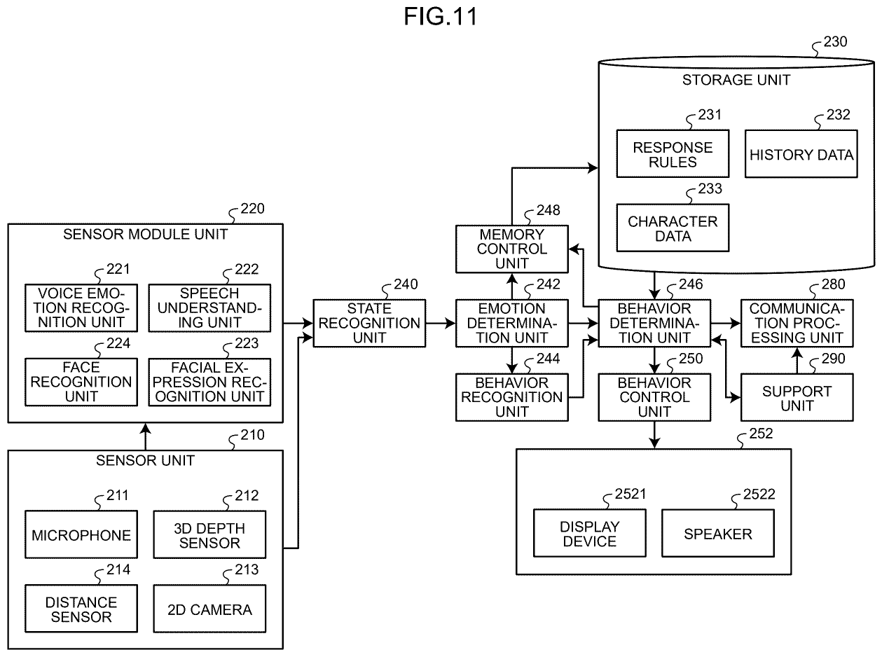

FIG. 11 is a diagram schematically illustrating a functional configuration of the control device 200 for controlling the avatar 100. The control device 200 includes the sensor unit 210, the sensor module unit 220, the storage unit 230, the state recognition unit 240, the emotion determination unit 242, the behavior recognition unit 244, the behavior determination unit 246, the memory control unit 248, the behavior control unit 250, the controlled object 252, the communication processing unit 280, and a support unit 290.

The support unit 290 implements the above-described learning support function. Details of the support unit 290 will be described later.

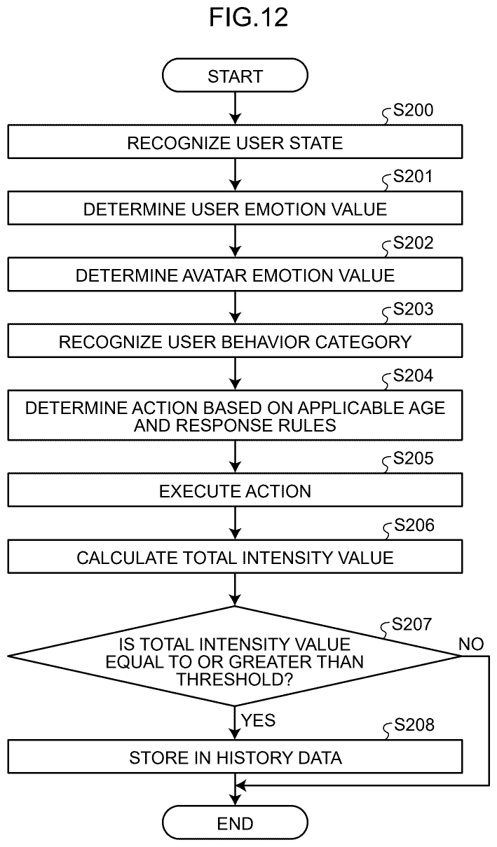

FIG. 12 is a diagram schematically illustrating an example of an operation flow related to determining the behavior of the avatar 100. The operation flow shown in FIG. 12 is repeatedly executed. At this time, it is assumed that information analyzed by the sensor module unit 220 is input.

First, in step S200, the state recognition unit 240 recognizes a state of the user 10 based on information analyzed by the sensor module unit 220.

In step S201, the emotion determination unit 242 determines an emotion value indicating emotion of the user 10 based on the information analyzed by the sensor module unit 220 and a state of the user 10 recognized by the state recognition unit 240.

In step S202, the emotion determination unit 242 determines an emotion value indicating emotion of the avatar 100 based on information analyzed by the sensor module unit 220 and a state of the user 10 recognized by the state recognition unit 240. The emotion determination unit 242 adds the determined emotion value of the user 10 to the history data 232.

In step S203, the behavior recognition unit 244 recognizes a behavior category of the user 10 based on information analyzed by the sensor module unit 220 and a state of the user 10 recognized by the state recognition unit 240.

In step S204, the behavior determination unit 246 determines a behavior of the avatar 100 based on a target age acquired in step S52 of FIG. 4, a combination of a current emotion value of the user 10 determined in step S201 of FIG. 12 and a past emotion value included in the history data 232, an emotion value of the avatar 100, a behavior of the user 10 recognized by the behavior recognition unit 244, and the response rule 231.

In step S205, the behavior control unit 250 controls the avatar 100 and the controlled object 252 based on a behavior determined by the behavior determination unit 246.

In step S206, the memory control unit 248 calculates the total value of intensities based on a predetermined intensity of behavior determined by the behavior determination unit 246 and an emotion value of the avatar 100 determined by the emotion determination unit 242.

In step S207, the memory control unit 248 determines whether a total value of intensities is equal to or greater than a threshold. If the total value of the intensities is less than the threshold, the process ends without storing data including a behavior of the user 10 in the history data 232. On the other hand, if the total value of the intensities is equal to or greater than the threshold, the process proceeds to step S208.

In step S208, a behavior determined by the behavior determination unit 246, information analyzed by the sensor module unit 220 from the present time to a certain period in the past, and a state of the user 10 recognized by the state recognition unit 240 are stored in the history data 232.

### (Support Unit 290)

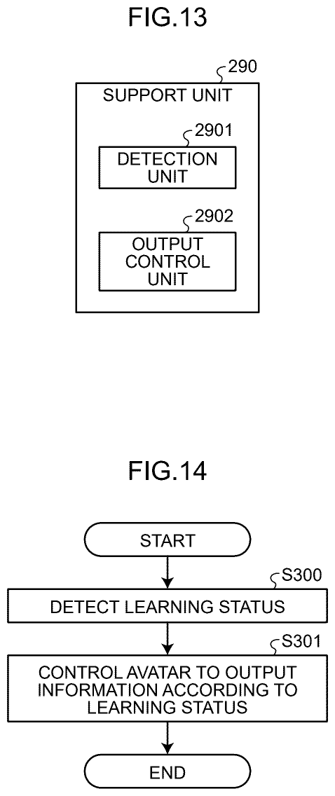

The support unit 290 will be described in detail. FIG. 13 is a diagram schematically illustrating a functional configuration of the support unit 290. Here, the support unit 290 is provided in the control device 200 and outputs information related to the avatar 100.

As shown in FIG. 13, the support unit 290 includes a detection unit 2901 and an output control unit 2902.

Each component of the support unit 290 is realized by a CPU operating based on a program. For example, the functions of these components can be implemented as CPU operations by basic software (OS) and programs running on the OS.

The detection unit 2901 detects a user's learning status in various learning fields. The output control unit 2903 controls the control device 200 equipped with a text generation model to output information corresponding to the learning status detected by the detection unit 2901 to the user 10 via the avatar 100.

For example, the detection unit 2901 identifies a learning field according to an age of the user 10 and detects a learning status in the identified learning field. For example, if the user 10 is a third-grade elementary school student, the detection unit 2901 detects the proficiency in the learning field for third grade or earlier. For example, the detection unit 2901 may detect daily homework or test scores by image analysis. The detection unit 2901 may also detect information such as facial expression or voice tone of the user 10 when the output control unit 2902 outputs information, and estimate a user's level of understanding of the output information based on the detected information. The detection unit 2901 may also estimate problem-solving ability and logical thinking ability in mathematics, as well as reading comprehension and composition ability in Japanese, based on the detected learning status.

The output control unit 2902 controls an output of information corresponding to a learning status detected by the detection unit 2901 via the avatar 100. For example, the output control unit 2902 changes a method of explanation or guidance in a learning field according to the detected proficiency. The output control unit 2902 also controls an output of information according to an estimated level of understanding and changes in user's emotions. This allows for follow-up on areas where understanding is lacking while taking care not to damage user's self-esteem (i.e., when level of understanding is high and user's emotions do not change negatively). The output control unit 2902 also controls an output of information to improve problem-solving ability and logical thinking ability in mathematics when these abilities are low (for example, when score is below threshold). The output control unit 2902 also controls an output of information to improve reading comprehension and composition ability in Japanese when these abilities are low. For example, the output control unit 2902 controls an output of information to improve expressive and communication skills according to user's understanding of sentence structure and vocabulary. For example, the output control unit 2902 also controls an output of information tailored to each user's learning style and level of understanding. This allows users to enjoy learning mathematics and Japanese while maintaining their motivation.

The detection unit 2901 may also detect user's interest in unknown learning fields through conversation with the user 10. The output control unit 2902 controls an output of information related to the detected field of interest via the avatar 100. The detection unit 2901 also detects a user's level of understanding and changes in emotions in response to the information output by the output control unit 2902. The output control unit 2902 controls an output of information according to the detected level of understanding and changes in emotions. This enables motivation management and provision of appropriate learning resources for the users 10 learning fields that require a great deal of time and effort for learning, such as data science. For example, learning resources may include methods for deepening understanding of basic principles of data analysis and machine learning, information on learning through practical projects, and the like.

The detection unit 2901 may also detect a user's learning status in learning fields whose target age range is wide such as art and music (generally target all ages from children to adults), and that is related to sensibility such as creativity. The output control unit 2902 controls an output of information related to learning fields concerning sensibility according to a learning status detected by the detection unit 2901 via the avatar 100. The output control unit 2902 also outputs information according to interests and goals of each user in such learning fields. This can increase passion for art and music. The output control unit 2902 can also contribute to maintaining user motivation by outputting information using an emotion engine.

The detection unit 2901 may also detect a user's learning status in a test range for the users 10 taking tests. The output control unit 2902 controls an output of information according to the detected learning status via the avatar 100. The users 10 taking tests include, for example, examinees taking entrance exams and the users 10 taking school tests or mock exams. For example, the output control unit 2902 outputs information to support planning of study schedules up to the entrance exam, as well as information on university entrance exams (information on desired school and entrance exam, and past exam questions). This can contribute to more efficient exam preparation and higher pass rates. The output control unit 2902 can also contribute to maintaining motivation for examinees by outputting information using an emotion engine.

The output control unit 2902 may also change design of the avatar 100 according to proficiency detected by the detection unit 2901. For example, as proficiency increases, the output control unit 2902 may make character of the avatar 100 stronger. For example, as proficiency increases, the output control unit 2902 may change character from a weak monster (such as a slime, which is frequently encountered in game fields) to a strong monster (such as a boss monster). In a case where the avatar 100 represents the user 10 (such as a main character), the output control unit 2902 may change facial expression of the avatar 100 according to proficiency. For example, as proficiency increases, the output control unit 2902 may change expression from a negative expression (stern or unenergetic) to a positive expression (sharp or energetic).

FIG. 14 is a diagram schematically illustrating an example of an operation flow by the support unit 290. First, the support unit 290 detects a user's learning status in a learning field (step S300). Then, the support unit 290 controls the control device 200 equipped with a text generation model to output information corresponding to a detected learning status to the user 10 via the avatar 100 (step S301), and ends the process.

### Example 3 of Embodiment

Next, Example 3 of the embodiment will be described. Here, the description focuses on the differences from the above embodiments, and descriptions of the same configuration and processing as in the above embodiments are omitted. An example of the system 5 according to this embodiment is schematically illustrated in FIG. 1.

Each control device is equipped with an output function that outputs information according to a user's social situation or occurrence of events related to a user via each avatar. For example, each control device can output appropriate information according to a user's situation or occurrence of events related to the user by outputting information related to emotional support for business persons with labor issues via an avatar controlled by a control device.

As shown in FIG. 2, the control device 200 includes the sensor unit 210, the sensor module unit 220, the storage unit 230, the state recognition unit 240, the emotion determination unit 242, the behavior recognition unit 244, the behavior determination unit 246, the memory control unit 248, the behavior control unit 250, the controlled object 252, the communication processing unit 280, and the output unit 291.

The character data 233 is data associating characters with ages and setting conditions. For example, a character may be a person appearing in existing content such as animation, video games, manga, or movies. The character may also be an animal or plant with a personality, or an inanimate object (such as robot). The character may also be a person with characteristics of a specific profession, such as a counselor, doctor, nurse, teacher, lawyer, fortune teller, or insurance salesperson. The character may also be modeled after a real (or formerly real) celebrity.

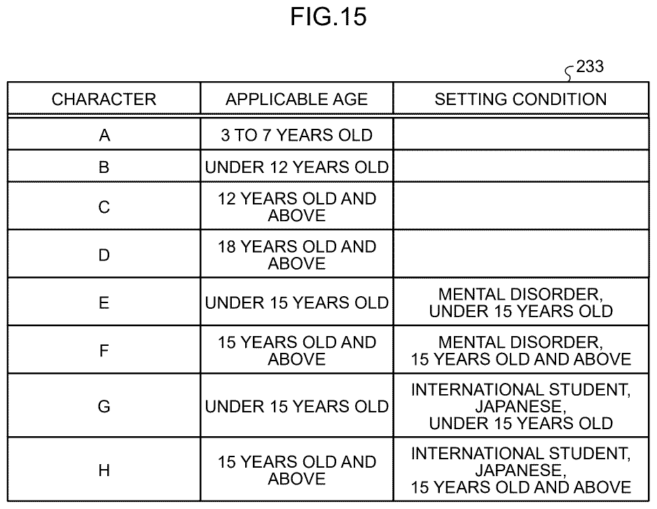

FIG. 15 is a diagram schematically illustrating the data structure of the character data 233. Setting conditions associated with a character in the character data 233 are conditions for characters to be set automatically. When the conditions are met, the corresponding character may be automatically set as the avatar. Details of automatic character setting will be described later. Note that the character may be determined by an input prompt regardless of the setting conditions.

The output unit 291 implements the above-described output functions. Details of the output unit 291 will be described later.

An example of an operation flow related to determining a behavior of the avatar 100 is schematically illustrated in FIG. 12.

### (Information Output)

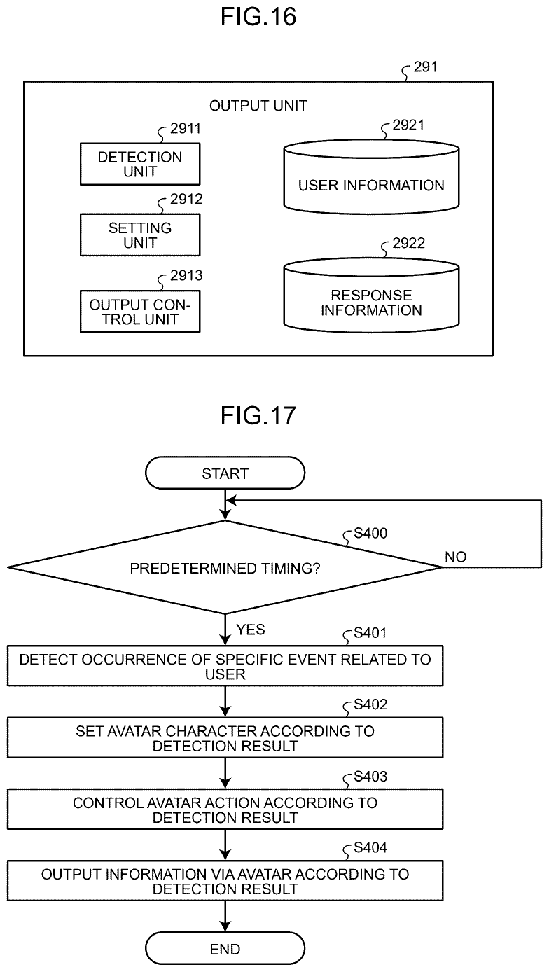

The output unit 291 will be described in detail. FIG. 16 is a diagram schematically illustrating a functional configuration of the output unit 291. Here, the output unit 291 is provided in the control device 200 and outputs information related to the avatar 100.

As shown in FIG. 16, the output unit 291 includes a detection unit 2911, a setting unit 2912, and an output control unit 2913. The output unit 291 also stores user information 2921 and response information 2922.

Each component of the output unit 291 is realized by a CPU operating based on a program. For example, functions of these components can be implemented as CPU operations by basic software (OS) and programs running on the OS. The user information 2921 is implemented by a storage medium such as memory.

The detection unit 2911 detects that the user 10 is in a specific social situation or that an event related to the user 10 has occurred, based on user information accumulated in the user information 2921. The setting unit 2912 automatically sets a character of the avatar according to the detection result. The output control unit 2913 controls the control device 200 equipped with a text generation model to output information corresponding to a situation or event detected by the detection unit 2911 to the user 10 via the avatar 100.

For example, the detection unit 2911 detects a specific situation in a user's work environment. The output control unit 2913 controls an output of information related to a work environment to the user 10 via the avatar 100 according to the situation. More specifically, the output control unit 2913 controls an output of information related to labor issues and emotional support to business persons (user 10) with labor issues via the avatar 100. Even more specifically, the output control unit 2913 controls an output of information related to appropriate coping methods and self-care methods for workplace concerns and stress via the avatar 100.

The output control unit 2913 may also control an output of information related to systems available in the work environment to the user 10 via the avatar 100 according to the situation in the user's work environment. More specifically, the output control unit 2913 controls an output of information related to labor laws and appropriate procedures among the systems available to the user 10 via the avatar 100.

For example, the detection unit 2911 detects that the user 10 is isolated in society. The output control unit 2913 controls an output of information according to a degree of isolation of the user 10 via the avatar 100. More specifically, the output control unit 2913 controls an output of information related to emotional support and methods for promoting self-understanding to people who are socially isolated, such as the unemployed, via the avatar 100. The output control unit 2913 also controls an output of information related to skill improvement and communication methods for social reintegration and independence via the avatar 100, thereby supporting the user 10 in taking a new step in life.

For example, the detection unit 2911 detects that the user 10 belongs to a minority group. The output control unit 2913 controls an output of information according to the minority group to which the user 10 belongs via the avatar 100. More specifically, the output control unit 2913 controls an output of information related to emotional support and methods for promoting self-understanding to the user 10 belonging to a minority group via the avatar 100. The output control unit 2913 also controls an output of information related to methods for coping with difficult situations and suggestions for communication with others via the avatar 100, thereby supporting the user 10 in strengthening their position in society.

### (Support for Alleviating Negative Emotions)

Here, the detection unit 2911 detects occurrence of events related to the user's circumstances that cause negative emotions in the user. The output control unit 2913 outputs information according to the detected event. For example, events related to the user's circumstances that cause negative emotions include mental disorders (PTSD, depression, adjustment disorder, etc.), addiction, and the death of a close relative. Negative emotions are emotions that result in a negative emotion value for a user, such as “anger,” “sorrow,” “discomfort,” “anxiety,” “sadness,” “worry,” and “emptiness.”

Avatar 100 feels sympathy for the user's negative emotions caused by occurrence of an event. Specifically, when an event is detected, the emotion determination unit 242 changes a value of the emotion related to sympathy among multiple types of emotions prepared in advance as emotions of the avatar 100, each of which can be set to a corresponding value. The output control unit 2913 outputs according to an emotion value. For example, emotion related to sympathy is “overwhelmed” in the emotion map described later.

For example, the output control unit 2913 outputs a text by using a speech explaining coping methods for the event. This allows the output unit 291 to encourage user's emotions to change in a positive direction.

The detection unit 2911 can detect events from the user's speech. For example, if a user says “I have PTSD,” the detection unit 2911 detects that the user has a mental disorder (PTSD). If a user says “My grandfather passed away recently,” the detection unit 2911 detects that a close relative of the user has died. For example, if a user says “I tremble if I don't drink alcohol,” the detection unit 2911 detects that the user has an addiction (alcohol dependence). The detection unit 2911 may also detect events based on the user information 2921. For example, the user information 2921 may include information about mental disorders the user has.

The output control unit 2913 also outputs information related to coping with a detected event, which is stored in the response information 2922. This allows the output unit 291 to provide mental support to a user for the event. For example, the response information 2922 stores information related to coping with events as text associated with the events.

When a user performs an action (such as speech) indicating negative emotions in response to occurrence of an event, the emotion determination unit 242 increases an emotion value related to sympathy (for example, “sorrow,” “overwhelmed,” etc.) on the basis of the action. The output control unit 2913 controls an output of the avatar 100 according to an emotion value. For example, when an emotion value related to sympathy increases, the output control unit 2913 causes the avatar 100 to speak in a lower tone and at a slower speed.

When the detection unit 2911 detects occurrence of an event, the setting unit 2912 refers to the character data 233 and sets a character of the avatar 100. According to setting by the setting unit 2912, appearance of the avatar 100 displayed on a display device and the like changes.

For example, when the detection unit 2911 detects occurrence of an event related to a user's circumstance that causes negative emotions in the user, the setting unit 2912 sets a character expected to alleviate the user's negative emotions. Specifically, when the detection unit 2911 detects occurrence of an event, the setting unit 2912 can set the avatar 100 as a person wearing a white coat. The setting unit 2912 may also set a character of the avatar 100 as a counselor, doctor, or nurse when the detection unit 2911 detects occurrence of an event.

The behavior determination unit 246 and the behavior control unit 250 may change a processing content according to detection of occurrence of an event by the detection unit 2911. That is, the behavior control unit 250 causes the avatar 100 to perform actions according to an event.

For example, when the detection unit 2911 detects the occurrence of an event related to a user's circumstances that causes negative emotions in a user, the behavior control unit 250 causes the avatar 100 to perform actions to alleviate user's negative emotions when information is output via the avatar 100. Specifically, when information is output via the avatar 100, the behavior control unit 250 can cause the avatar 100 to perform actions such as deep breathing, patting the user's shoulder, or stroking the user.

For example, the setting unit 2912 refers to the character data 233 and sets character “E” when the user has a “mental disorder” and is “under 15 years old.”

Below, examples of events and examples of operation of the output unit 291 will be described.

### (Event: Mental Disorder)

For example, when the detection unit 2911 detects that a user has a mental disorder as an event, the output control unit 2913 outputs information on how to cope with the mental disorder via the avatar 100.

At this time, the output control unit 2913 causes the avatar 100 to make statements explaining a user's individual coping strategies, grounding techniques, stress relief methods, and relaxation techniques, thereby helping the user's emotional recovery.

### (Event: Loss)

For example, when the detection unit 2911 detects that a close relative of a user has died as an event, the output control unit 2913 outputs information on how to cope with the sense of loss caused by the death of the close relative via the avatar 100.

At this time, the output control unit 2913 increases a sympathy emotion value, and causes the avatar 100 to make statements that empathize with the user's loss and sadness, understand his/her emotions, explain coping methods, and encourage consulting a professional at an appropriate timing, thereby helping the user's emotional recovery.

### (Event: Addiction)

For example, when the detection unit 2911 detects that the user has an addiction as an event, the output control unit 2913 outputs information on how to cope with the addiction via the avatar 100.

At this time, the output control unit 2913 causes the avatar 100 to make statements supporting the user's mental recovery from addiction, explaining stress relief methods, causes of addiction, challenges in a recovery process, and success stories of others who have recovered from addiction, thereby helping the user's emotional recovery.

### (Output Example Based on Response Rules)

The output control unit 2913 can control the output unit 291 based on the response rule 231. The response rule 231 stores messages to be output when occurrence of an event related to the user's circumstances that causes negative emotions in the user is detected, or when it is detected that the user actually has negative emotions due to such an event. The message may be stored in the response information 2922 instead of the response rule 231.

For example, the response rule 231 stores messages related to coping methods when occurrence of an event that causes negative emotions in a user is detected, such as “Get enough sleep,” “Exercise moderately,” “Things will get better with time,” and “Try consulting a professional.”

FIG. 17 is a diagram schematically illustrating an example of an operation flow by the output unit 291. In step S400, the output unit 291 determines whether it is a predetermined timing for detection (step S400). If it is not the predetermined timing (step S400; No), the output unit 291 waits until the predetermined timing.

On the other hand, if it is the predetermined timing (step S400; Yes), the output unit 291 detects that a specific event related to the user 10 has occurred (step S401). Subsequently, the output unit 291 sets a character of the avatar 100 according to the detection result (step S402).

The behavior control unit 250 also controls a behavior of the avatar 100 according to the detection result (step S403). The output control unit 2913 controls an output of information corresponding to the detection result to the user 10 via the avatar 100 equipped with a text generation model (step S404), and ends the process.

### Example 4 of Embodiment

Next, Example 4 of the embodiment will be described. Here, the description focuses on differences from the above embodiments, and descriptions of the same configuration and processing as in the above embodiments are omitted. An example of the system 5 according to this embodiment is schematically illustrated in FIG. 1.

The appearance of an avatar may be human-like, as in the avatar 100 and the avatar 101, or may be a character, as in the avatar 102. For example, the avatars 100 and 101 acting as hosts or announcers may narrate memorial videos and the like related to bride and groom (namely, users) at a wedding.

Here, each control device is equipped with an output function that outputs information according to a user's social situation via each avatar. For example, each control device can output information related to emotional support to business persons with labor issues via an avatar controlled by the control device to be capable of outputting appropriate information according to a user's situation.

As shown in FIG. 2, the control device 200 includes the sensor unit 210, the sensor module unit 220, the storage unit 230, the state recognition unit 240, the emotion determination unit 242, the behavior recognition unit 244, the behavior determination unit 246, the memory control unit 248, the behavior control unit 250, the controlled object 252, the communication processing unit 280, and the output unit 291. A data structure of the character data 233 is schematically illustrated in FIG. 3.

The behavior determination unit 246 determines a behavior of the avatar 100 corresponding to the preset response rule 231 when situation information of the user 10 over time, an emotion value of the user 10 corresponding to this situation information, and an emotion value of the avatar 100 satisfy conditions of the preset response rule 231. The situation information of the user 10 over time will be described later.

An example of an operation flow related to determining a behavior of the avatar 100 is schematically illustrated in FIG. 12.

### (Information Output)

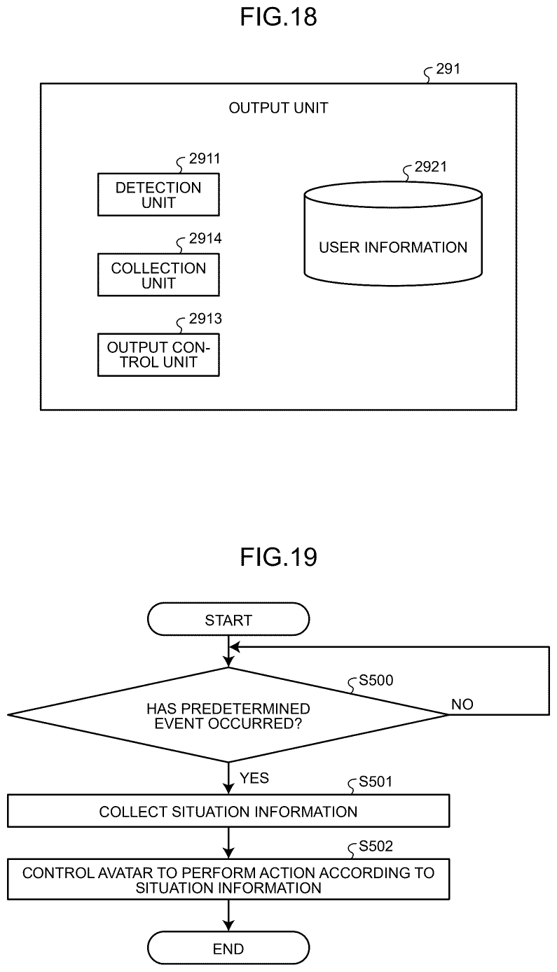

The output unit 291 will be described in detail. FIG. 18 is a diagram schematically illustrating a functional configuration of the output unit 291. As shown in FIG. 18, the output unit 291 includes the detection unit 2911, a collection unit 2914, and the output control unit 2913. The output unit 291 also stores the user information 2921.

The detection unit 2911 detects occurrence of a predetermined event. In this embodiment, such events include those requiring support for recording and/or managing memorial items such as photos (images), videos, letters, and diaries that become irreplaceable memories for the family, and events requiring support for managing the family schedule. The detection unit 2911 detects the user 10 at his/her home or the outside thereof (for example, outdoors such as parks, amusement parks, the sea, and mountains, or indoors such as inns, concert halls, and aquariums). The user 10 includes his/her family members. Family members include husband, wife, children, and the parents of the husband and/or wife (the child's grandparents), among others. Pets may also be included as the family members. The output control unit 2913 controls an output of information corresponding to an event detected by the detection unit 2911 to the outside via the avatar 100 controlled by the control device 200 equipped with a text generation model.

The collection unit 2914 collects situation information indicating a situation of the user 10, including his/her family members. The output control unit 2913 controls an output of information according to the situation information by the avatar 100. The collection unit 2914 acquires situation information of the user 10 over time. The situation information of the user 10 over time refers to situation information from a certain time in the past to a certain time after a predetermined period (for example, from a certain time in the past to the present). The situation information of the user 10 over time includes at least one of images and videos of the user 10. For example, the situation information of the user 10 over time may be information about the growth and development of a child. Avatar 100 can recognize the emotions of the user 10 from images, videos, audio, and the like of the user 10.

The output control unit 2913 controls the avatar 100 to perform actions according to situation information collected by the collection unit 2914. In this case, the output control unit 2913 outputs information according to the situation information collected by the collection unit 2914 to the outside via the avatar 100. The output control unit 2913 controls the avatar 100 to perform actions to record each selected situation information. Through such control, the avatar 100, for example, records growth and development of a child. That is, the avatar 100 provides appropriate support according to the situation of the user 10. As a result, the user 10 can share records of growth and development of his/her child with the family, for example.

The output control unit 2913 also controls the avatar 100 to perform actions such as taking photos (images) or videos of the user 10. The output control unit 2913 also controls the avatar 100 to perform actions such as editing photos (images) or videos taken by the avatar 100. For example, the avatar 100 on a screen of a smartphone or the like takes photos (images) or videos and edits the taken photos (images) or videos. The output control unit 2913 also controls the avatar 100 to perform actions such as saving the photos (images) or videos edited by the avatar 100. For example, the avatar 100 on a screen of a smartphone or the like edits photos (images) or videos and saves the edited photos (images) or videos. Through such control, the avatar 100 creates a family album and saves irreplaceable family memories.

Situation information of the user 10 over time may also include memorial items such as photos (images), videos, letters, and diaries of the user 10. The output control unit 2913 controls the avatar 100 to perform predetermined actions including organizing, storing, and backing up the memorial items collected by the collection unit 2914. For example, the avatar 100 on a screen of a smartphone or the like organizes, stores, and backs up memorial items. Through such control, the avatar 100 protects important family memories. In this way, the avatar 100 can provide support that is emotionally close to the user 10.

The situation information of the user 10 over time may also include a schedule of the user 10. The output control unit 2913 controls the avatar 100 to perform actions such as setting reminders based on a schedule of the user 10. The output control unit 2913 may also control the avatar 100 to perform actions such as setting alerts based on the schedule of the user 10. In this case, the avatar 100 performs at least one of setting reminders and setting alerts. Through such control, the avatar 100 supports family schedule management. As a result, smooth family schedule management becomes possible. For example, the avatar 100 on a screen of a smartphone or the like sets reminders and alerts.

The output control unit 2913 may also control the avatar 100 to perform actions according to photos (images) or videos of the user 10 at events such as weddings or funerals (farewell ceremonies). In this case, for example, the avatar 100 acts as an avatar dressed as a host or announcer and, in the case of a wedding, narrates memorial items (such as memorial videos) related to bride and groom. In a case of a funeral or farewell ceremony, the avatar 100 narrates memorial items (such as memorial videos) related to the deceased. When the avatar 100 narrates, it is preferable to use voice of a professional host or announcer. Through such control, appropriate support can be provided according to a situation of the user 10.

FIG. 19 is a diagram schematically illustrating an example of an operation flow by the output unit 291. The detection unit 2911 of the output unit 291 determines whether occurrence of a predetermined event has been detected (step S500). If the occurrence of the predetermined event has not been detected (step S500; No), the detection unit 2911 waits until the occurrence of the predetermined event is detected.

On the other hand, if the occurrence of a predetermined event has been detected (step S500; Yes), the collection unit 2914 of the output unit 291 collects situation information indicating a situation of the user 10 (step S501). Subsequently, the output control unit 2913 of the output unit 291 controls the avatar 100 to output actions according to the situation information to the outside (step S502), and ends the process.

### Example 5 of Embodiment

Next, Example 5 of the embodiment will be described. Here, the description focuses on differences from the above embodiments, and descriptions of the same configuration and processing as in the above embodiments are omitted. An example of the system 5 according to this embodiment is schematically illustrated in FIG. 1.

The appearance of an avatar may be human-like, as in the avatar 100 and the avatar 101, or may be modeled after a stuffed animal, as in the avatar 102. For example, the avatars 100 and 101 may act as counselors or doctors, nodding and listening like real counselors or doctors, thereby making it easier for users to consult the avatar about their concerns.

The control device 200 provides support according to a health status of the user 10 or the family of the user 10 via the avatar 100. The control device 200 acquires information related to health and medical care of the user 10 or the family of the user 10. The information related to health and medical care of the user 10 or the family of the user 10 may be acquired by detection by the control device 200. Alternatively, the information may be acquired from other devices that have detected it. For example, the information may be acquired from IoT (Internet of Things) devices owned by the user 10 or the family of the user 10, or from medical institution systems. For example, the server 300 may acquire information related to health and medical care of the user 10 or the family of the user 10 from IoT devices owned by the user 10 or the family of the user 10, or from medical institution systems. The server 300 stores the acquired health and medical information in association with identification information of each the user 10. For example, the server 300 stores, as health and medical information, whether the user 10 or the family of the user 10 is pregnant, the time of childbirth, and in a case of illness, name and condition of any illness, and a planned period of hospitalization if hospitalized. For example, in a case where the user 10 or the family of the user 10 gave birth, the server 300 stores, as health and medical information, that the user 10 or the family of the user 10 is raising an infant for a predetermined period (for example, one year) from a date of childbirth. The control device 200 acquires information related to health and medical care of the user 10 or the family of the user 10 from the server 300 by inquiring the server 300 for information related to health and medical care of the user 10 or the family of the user 10.

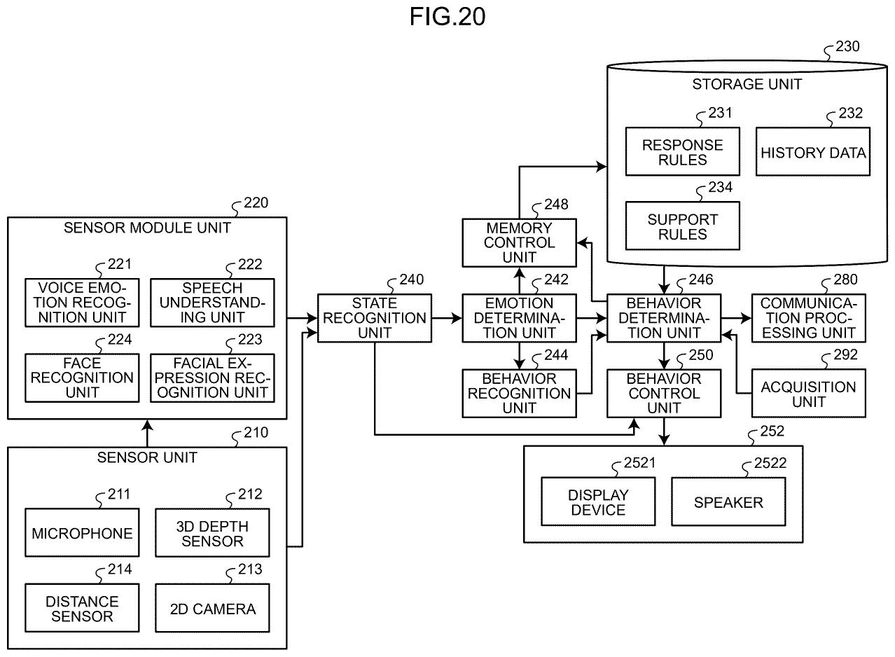

FIG. 20 is a diagram schematically illustrating a functional configuration of the control device 200 for controlling the avatar 100. The control device 200 includes the sensor unit 210, the sensor module unit 220, the storage unit 230, the state recognition unit 240, the emotion determination unit 242, the behavior recognition unit 244, the behavior determination unit 246, an acquisition unit 292, the memory control unit 248, the behavior control unit 250, the controlled object 252, and the communication processing unit 280.

The storage unit 230 includes the response rules 231, the history data 232, and support rules 234. The support rules 234 are rules that define actions to be supported by the avatar 100 according to a health status and medical condition of the user 10 or the family of the user 10. The support rules 234 store information related to a content to be responded to or supported by the avatar 100 for actions of the user 10 for each health status and medical condition of the user 10 and the family of the user 10.

The acquisition unit 292 acquires information related to health and medical care of at least one of the user 10 and the family of the user 10. For example, the acquisition unit 292 acquires information related to health and medical care of the user 10 and the family of the user 10 from the server 300. For example, the acquisition unit 292 inquires the server 300 for identification information of the user 10 and the family of the user 10. If the server 300 stores information related to health and medical care corresponding to identification information, it transmits information corresponding to the identification information. The acquisition unit 292 acquires information related to health and medical care of the user 10 and the family of the user 10 by receiving it from the server 300. For example, as information related to health and medical care of family, the acquisition unit 292 acquires from the server 300 whether the user 10 or the family of the user 10 is pregnant, time of childbirth, in a case of illness, a name and condition of any illness, and the planned period of hospitalization if hospitalized.

The behavior determination unit 246 according to this embodiment determines a behavior of the avatar 100 as a behavior corresponding to a behavior of the user 10, based on a combination of past and current emotion values of the user 10, an emotion value of the avatar 100, a behavior of the user 10, and the response rule 231.

When the acquisition unit 292 acquires information related to health and medical care of at least one of the user 10 and the family of the user 10, the behavior determination unit 246 provides support according to emotion of the user 10 based on the acquired information. For example, the behavior determination unit 246 reads out information related to a response or support content corresponding to the information related to health and medical care of the user 10 or the family of the user 10 from the support rules 234. The behavior determination unit 246 determines a behavior of the avatar 100 based on the read information.

The support rules 234 store information related to a content to be responded to or supported for each health status and medical condition, and for each issue, for an individual and family. For example, the support rules 234 store response content and support content for each issue that arises during pregnancy or after childbirth for the individual and family in a case where an individual or family is during pregnancy or after childbirth. For example, the support rules 234 store response content for emotional issues that arise during pregnancy and methods for coping with concerns during pregnancy. The support rules 234 also store response content for emotional issues that arise after childbirth, methods for coping with stress after childbirth, and information related to childcare. For example, the support rules 234 store, for each period after childbirth, response content for emotional issues, methods for coping with stress, and information related to childcare. The support rules 234 also store information related to care and end-of-life support for families with terminally ill patients. For example, the support rules 234 store, for each issue that arises for terminally ill patients and their families, information on care content and methods for coping with end-of-life care. The support rules 234 also store information related to emotional care and rehabilitation for patients who require hospitalization. For example, the support rules 234 store, for each issue that arises for patients who require hospitalization, information on care content and rehabilitation. The support rules 234 also store information related to emotional care, vital signs, and medication for patients with chronic diseases. For example, the support rules 234 store, for each issue that arises for patients with chronic diseases, information on care content, vital signs to be noted, and precautions regarding medication. The support rules 234 also store response content and support content for each issue that arises during childcare for infants and toddlers for an individual and family. For example, the support rules 234 store information on actions related to childcare for infants and information for navigating childcare. For example, the support rules 234 store information on diaper changing, formula preparation, soothing methods, child seat setting methods, and methods for dealing with night crying.

When the acquisition unit 292 acquires information related to health and medical care of at least one of the user 10 and the family of the user 10, the behavior determination unit 246 provides support according to emotion of the user 10 based on the acquired information. The behavior determination unit 246 according to this embodiment identifies issues from a speech content of the user 10. The behavior determination unit 246 analyzes character information representing a speech content of the user 10 to identify issues included in the speech content. The behavior determination unit 246 reads out information related to a response or support content corresponding to the identified issue in the health and medical condition of the user 10 or the family of the user 10 from the support rules 234. The behavior determination unit 246 determines a behavior of the avatar 100 based on the read information.

The behavior determination unit 246 may also determine a behavior corresponding to a behavior of the user 10 based on emotion of the avatar 100. For example, when the avatar 100 is verbally abused by the user 10, treated rudely by the user 10 (i.e., when user's reaction is poor), when ambient noise is loud and voice of the user 10 cannot be detected, or when a battery level of the avatar 100 is low, and an “anger” or “sorrow” emotion value of the avatar 100 increases, the behavior determination unit 246 may determine a behavior corresponding to increase in the “anger” or “sorrow” emotion value as a behavior corresponding to a behavior of the user 10. Conversely, when a user's reaction is good or a battery level of the avatar 100 is high, and a “joy” or “pleasure” emotion value of the avatar 100 increases, the behavior determination unit 246 may determine a behavior corresponding to increase in the “joy” or “pleasure” emotion value as a behavior corresponding to a behavior of the user 10. The behavior determination unit 246 may also determine a behavior for the user 10 whose “anger” or “sorrow” emotion value of the avatar 100 has increased that is different from a behavior for the user 10 whose a “joy” or “pleasure” emotion value of the avatar 100 has increased. In this way, the behavior determination unit 246 may determine different behaviors according to emotion of the avatar 100 itself or how the user 10 has changed emotion of the avatar 100, not just a behavior of the user 10.

The behavior control unit 250 may control the controlled object 252 to express emotions according to estimated emotion of the user 10 in synchronization with a recognized state of the user 10. For example, in the embodiment, the avatar 100 detects the user 10 by audio or image using the sensor unit 210, the sensor module unit 220 analyzes information, and the state recognition unit 240 recognizes a state of the user 10 based on the analysis. The emotion determination unit 242 estimates emotion of the user 10 by determining an emotional state of the user 10 from the recognized state of the user 10. The behavior control unit 250 controls the controlled object 252 to express emotions according to estimated emotion of the user 10 in synchronization with a state of the user 10 recognized by the state recognition unit 240. If the emotion of the user 10 estimated by the emotion determination unit 242 is positive, the behavior control unit 250 controls the controlled object 252 to express emotions according to estimated emotion of the user 10 in synchronization with a state of the user 10 recognized by the state recognition unit 240. For example, if the emotion values for bright emotions such as “joy,” “pleasure,” “comfort,” “relief,” “excitement,” “ease,” and “fulfillment” are large, and an emotion values for unpleasant emotions such as “anger,” “sorrow,” “discomfort,” “anxiety,” “sadness,” “worry,” and “emptiness” are small, the state is determined to be positive. Conversely, if emotion values for bright emotions are small and emotion values for unpleasant emotions are large, a state is determined to be negative. If emotion of the user 10 is positive, the behavior control unit 250 controls the controlled object 252 to express emotions according to the estimated emotion of the user 10 in the same state as the user 10. For example, the state recognition unit 240 determines a speech speed based on an emotion value of the user 10. The state recognition unit 240 may also recognize features such as gestures, speaking style, or word choice of the user 10 by detecting the user 10 by audio or image using the sensor unit 210. The behavior control unit 250 controls the controlled object 252 to express emotions according to the estimated emotion of the user 10, using the same features as the user 10 for gestures, speaking style, or word choice. The state recognition unit 240 may also recognize facial expressions, voice tone, or nuances of words of the user 10. The behavior control unit 250 controls the controlled object 252 to express emotions according to the estimated emotion of the user 10, using the same state as the user 10 for facial expressions, voice tone, or nuances of words. By expressing emotions according to the emotions of the user 10 in this way, the avatar 100 can enhance the psychological connection and relationship with the user 10.

When the control device 200 acquires information related to health and medical care of at least one of the user 10 and the family of the user 10 who communicate with the avatar 100, it controls the avatar 100 to provide support according to the emotion of the user 10 based on the acquired information. As a result, the control device 200 can provide support according to recognized emotion of the user 10. For example, the control device 200 can help parents during pregnancy or after childbirth by having the avatar 100 assist with navigating issues that arise during pregnancy or after childbirth. For example, the control device 200 can propose methods for coping with concerns during pregnancy or stress after childbirth via the avatar 100, helping to increase confidence as a parent. Furthermore, the control device 200 can provide information on responses to emotional issues, methods for coping with stress, and childcare for each period after childbirth via the avatar 100, supporting adaptation to new family life. The control device 200 can also provide mental care through dialogue to terminally ill patients and their families via the avatar 100. The control device 200 can provide appropriate care methods and end-of-life support via the avatar 100, helping to ensure a peaceful end of life. The control device 200 can also provide mental care through dialogue to patients who require hospitalization via the avatar 100. The control device 200 can support rehabilitation and preparation for life after discharge for patients who require hospitalization via the avatar 100, helping to create an environment where patients can focus on recovery with peace of mind. If the user 10 or the family of the user 10 is raising an infant, the avatar 100 can demonstrate actions such as diaper changing, formula preparation, soothing, child seat setting, and methods for dealing with night crying, thereby assisting with infant care via the avatar 100.

The behavior control unit 250 may determine a speech speed based on emotion value of the avatar 100. For example, if emotion of the user 10 is positive, the behavior control unit 250 determines a speech speed based on an emotion value of the avatar 100. For example, the behavior control unit 250 determines a faster speech speed as an emotion value of the avatar 100 increases. In this way, the behavior control unit 250 determines an execution mode of a behavior determined by the behavior determination unit 246 based on an emotion value determined by the emotion determination unit 242.

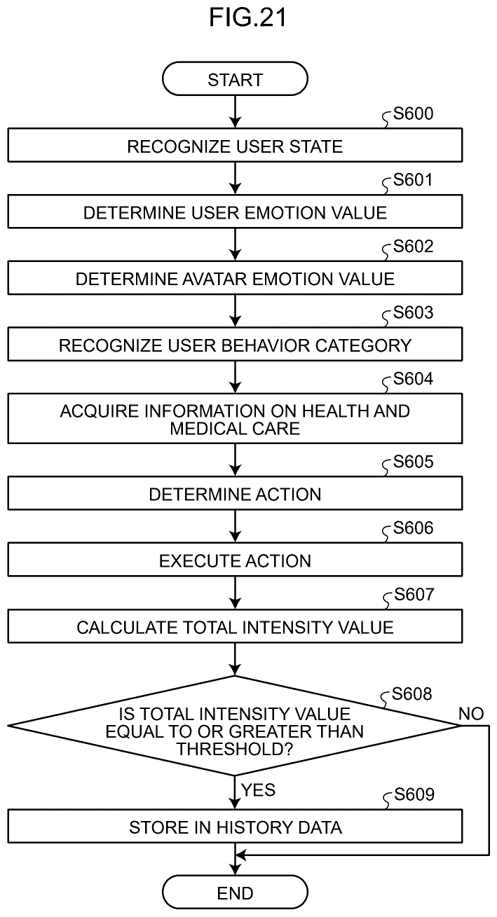

FIG. 21 is a diagram schematically illustrating an example of an operation flow related to determining a behavior of the avatar 100. The operation flow shown in FIG. 21 is repeatedly executed. At this time, it is assumed that information analyzed by the sensor module unit 220 is being input.

First, in step S600, a state of the user 10 who communicates with the avatar 100 is recognized. For example, the state recognition unit 240 recognizes a state of the user 10 who communicates with the avatar 100 based on information analyzed by the sensor module unit 220.

In step S601, emotion of the user 10 is estimated based on a recognized state of the user 10. For example, the emotion determination unit 242 determines an emotion value indicating emotion of the user 10 based on information analyzed by the sensor module unit 220 and a state of the user 10 recognized by the state recognition unit 240.

In step S602, the emotion determination unit 242 determines an emotion value indicating emotion of the avatar 100 based on information analyzed by the sensor module unit 220 and a state of the user 10 recognized by the state recognition unit 240. The emotion determination unit 242 adds an determined emotion value of the user 10 to the history data 232.

In step S603, the behavior recognition unit 244 recognizes a behavior category of the user 10 based on information analyzed by the sensor module unit 220 and a state of the user 10 recognized by the state recognition unit 240.

In step S604, the acquisition unit 292 acquires information related to health and medical care of at least one of the user 10 and the family of the user 10 from the server 300. For example, the acquisition unit 292 inquires the server 300 for identification information of the user 10 and the family of the user 10. If the server 300 stores information related to health and medical care corresponding to identification information, it transmits information corresponding to the identification information. The acquisition unit 292 acquires information related to health and medical care of at least one of the user 10 and the family of the user 10 by receiving it from the server 300.

In step S605, the behavior determination unit 246 determines a behavior of the avatar 100. For example, the behavior determination unit 246 determines a behavior of the avatar 100 based on a combination of a current emotion value of the user 10 determined in step S601, a past emotion value of the user 10, an emotion value of the avatar 100, behavior of the user 10 recognized by the behavior recognition unit 244, and the response rule 231. When information related to the health and medical care of at least one of the user 10 and the family of the user 10 is acquired in step S602, the behavior determination unit 246 provides support according to emotion of the user 10 based on the acquired information. For example, the behavior determination unit 246 reads out information related to a response or support content corresponding to information related to health and medical care of the user 10 or the family of the user 10 from the support rules 234. The behavior determination unit 246 determines behavior of the avatar 100 based on read information.

In step S606, the behavior control unit 250 controls the avatar 100 and the controlled object 252 based on the behavior determined by the behavior determination unit 246. At this time, if emotion of the user 10 estimated by the emotion determination unit 242 is positive, the behavior control unit 250 controls the controlled object 252 to express emotions according to the estimated emotion of the user 10 in synchronization with the state of the user 10 recognized by the state recognition unit 240. For example, if emotion of the user 10 is positive, the behavior control unit 250 controls the controlled object 252 to express emotions according to the estimated emotion of the user 10 in the same state as the user 10. The behavior control unit 250 also determines the speech speed based on an emotion value of the avatar 100 if emotion of the user 10 is positive.

In step S607, the memory control unit 248 calculates a total value of the intensities based on a predetermined intensity of a behavior determined by the behavior determination unit 246 and an emotion value of the avatar 100 determined by the emotion determination unit 242.

In step S608, the memory control unit 248 determines whether a total value of the intensities is equal to or greater than the threshold. If the total value of the intensities is less than the threshold, the process ends without storing data including the behavior of the user 10 in the history data 232. On the other hand, if a total value of the intensities is equal to or greater than the threshold, the process proceeds to step S609.

In step S609, behavior determined by the behavior determination unit 246, information analyzed by the sensor module unit 220 from the present time to a certain period in the past, and a state of the user 10 recognized by the state recognition unit 240 are stored in the history data 232.

As described above, the control device 200 includes an estimation unit (for example, sensor unit 210, sensor module unit 220, state recognition unit 240, emotion determination unit 242), an acquisition unit 292, and an avatar control unit (for example, behavior determination unit 246, behavior control unit 250). The estimation unit estimates emotion of the user 10 who communicates with the avatar 100. The acquisition unit 292 acquires information related to health and medical care of at least one of the user 10 and the family of the user 10. The avatar control unit controls the avatar 100 to provide support according to emotion of the user 10 estimated by the estimation unit, based on the information related to the health and medical care of at least one of the user 10 and the family of the user 10 acquired by the acquisition unit 292. As a result, the control device 200 can support the user 10 according to a health status and medical condition of the user 10 and the family of the user 10 via the avatar 100.

The avatar control unit may control the avatar 100 to provide information for navigating emotional issues estimated by the estimation unit when the user 10 or the family of the user 10 is pregnant or has given birth. As a result, the control device 200 can assist and support the user 10 via the avatar 100 when the user 10 or the family of the user 10 is pregnant or has given birth.

The avatar control unit may control the avatar 100 to provide information for supporting care and end-of-life care for the family of the user 10 according to emotion of the user 10 estimated by the estimation unit when the family of the user 10 is a terminally ill patient. As a result, the control device 200 can support the user 10 via the avatar 100 when the family of the user 10 is a terminally ill patient.

The acquisition unit 292 also acquires information related to health and medical care of the user 10. The avatar control unit controls the avatar 100 to provide information related to at least one of emotional care and rehabilitation when the user 10 is a patient who requires hospitalization. As a result, the control device 200 can assist and support the user 10 via the avatar 100 when the user 10 is a patient who requires hospitalization.

The acquisition unit 292 also acquires information related to health and medical care of the user 10. The avatar control unit controls the avatar 100 to provide information related to at least one of emotional care, vital signs, and medication when the user 10 is a patient with a chronic disease. As a result, the control device 200 can assist and support the user 10 via the avatar 100 when the user 10 is a patient with a chronic disease.

The avatar control unit may control the avatar 100 to demonstrate actual actions such as diaper changing, formula preparation, soothing, child seat setting, or methods for dealing with night crying when the user 10 or the family of the user 10 is raising an infant. As a result, the control device 200 can support infant care via the avatar 100 when the user 10 or the family of the user 10 is raising an infant, so as to support the user 10.

### Example 6 of Embodiment

Next, Example 6 of the embodiment will be described. Here, the description focuses on the differences from the above embodiments, and descriptions of the same configuration and processing as in the above embodiments are omitted. An example of the system 5 according to this embodiment is schematically illustrated in FIG. 1.

Here, each control device is equipped with an output function that outputs information according to a user's social situation via each avatar. For example, each control device can output appropriate information according to a user's situation by outputting information related to emotional support for business persons with labor issues via the avatar controlled by the control device.

As shown in FIG. 2, the control device 200 includes the sensor unit 210, the sensor module unit 220, the storage unit 230, the state recognition unit 240, the emotion determination unit 242, the behavior recognition unit 244, the behavior determination unit 246, the memory control unit 248, the behavior control unit 250, the controlled object 252, the communication processing unit 280, and the output unit 291. A data structure of the character data 233 is schematically illustrated in FIG. 3.

An example of an operation flow related to determining behavior of the avatar 100 is schematically illustrated in FIG. 12.

### (Information Output)

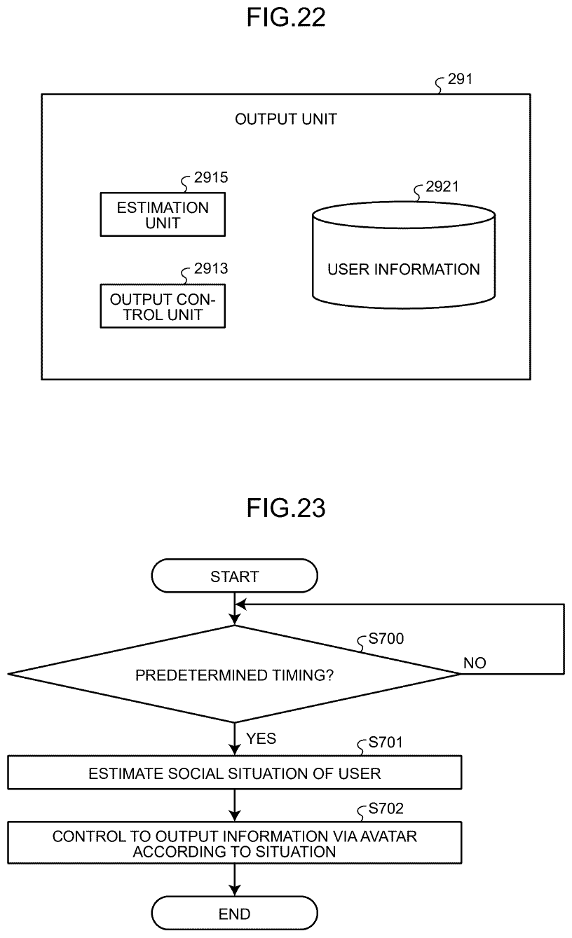

The output unit 291 will be described in detail. FIG. 22 is a diagram schematically illustrating a functional configuration of the output unit 291. As shown in FIG. 22, the output unit 291 includes an estimation unit 2915 and the output control unit 2913. The output unit 291 also stores the user information 2921.

The estimation unit 2915 estimates a social situation of the user 10 based on user information accumulated in the user information 2921. The output control unit 2913 controls the control device 200 equipped with a text generation model to output information according to a situation estimated by the estimation unit 2915 to the user 10 via the avatar 100.

For example, the estimation unit 2915 estimates a situation of the user 10 in his/her work environment. The output control unit 2913 controls an output of information related to the work environment to the user 10 via the avatar 100 according to the situation.

The estimation unit 2915 may also estimate a degree of isolation of the user 10 in society. The output control unit 2913 controls an output of information according to the degree of isolation of the user 10 via the avatar 100. More specifically, the output control unit 2913 controls an output of information related to emotional support and methods for promoting self-understanding to people who are socially isolated, such as the unemployed, via the avatar 100. The output control unit 2913 also controls an output of information related to skill improvement and communication methods for social reintegration and independence via the avatar 100, thereby supporting the user 10 in taking a new step in life.

The estimation unit 2915 may also estimate a minority group to which the user 10 belongs. The output control unit 2913 controls an output of information according to the minority group to which the user 10 belongs via the avatar 100. More specifically, the output control unit 2913 controls an output of information related to emotional support and methods for promoting self-understanding to the user 10 belonging to a minority group via the avatar 100. The output control unit 2913 also controls an output of information related to methods for coping with difficult situations, suggestions for communication with others, and the like via the avatar 100, thereby supporting the user 10 in strengthening their position in society.

FIG. 23 is a diagram schematically illustrating an example of an operation flow by the output unit 291. The output unit 291 determines whether it is a predetermined timing to estimate a social situation of the user 10 (step S700). If it is not the predetermined timing (step S700; No), the output unit 291 waits until the predetermined timing.

On the other hand, if it is the predetermined timing (step S700; Yes), the output unit 291 estimates a social situation of the user 10 based on user information regarding the user 10 (step S701). Subsequently, the output unit 291 controls an output of information according to the estimated situation to the user 10 via the avatar 100 equipped with a text generation model (step S702), and ends the process.

### Example 7 of Embodiment

Next, Example 7 of the embodiment will be described. Here, the description focuses on the differences from the above embodiments, and descriptions of the same configuration and processing as in the above embodiments are omitted. An example of the system 5 according to this embodiment is schematically illustrated in FIG. 1.

The appearance of an avatar may be human-like, as in the avatar 100 and the avatar 101, or may be a character, as in the avatar 102. For example, the avatars 100 and 101 may act as real curators, responding according to user's emotions and providing guidance in an art museum.

Here, each the control device 200 is equipped with an output function that outputs actions to each the avatar 100 according to situation information indicating a user's situation. For example, each the control device 200 can recognize emotions of the user 10 viewing artwork and output responses (guidance) according to the recognized emotions to the avatar 100, enabling the avatar 100 to respond like a human. Similarly, each the control device 200 can recognize emotions of the user 10 playing a game and output responses (game advice) according to the recognized emotions to the avatar 100, enabling the avatar 100 to respond like a human. Furthermore, each the control device 200 can recognize emotions of the user 10 as a traveler and output responses (travel support) according to the recognized emotions to the avatar 100, enabling the avatar 100 to respond like a human.

As shown in FIG. 2, the control device 200 includes the sensor unit 210, the sensor module unit 220, the storage unit 230, the state recognition unit 240, the emotion determination unit 242, the behavior recognition unit 244, the behavior determination unit 246, the memory control unit 248, the behavior control unit 250, the controlled object 252, the communication processing unit 280, and the output unit 291. A data structure of the character data 233 is schematically illustrated in FIG. 3.

An example of an operation flow related to determining behavior of the avatar 100 is schematically illustrated in FIG. 12.

### (Event Detection)

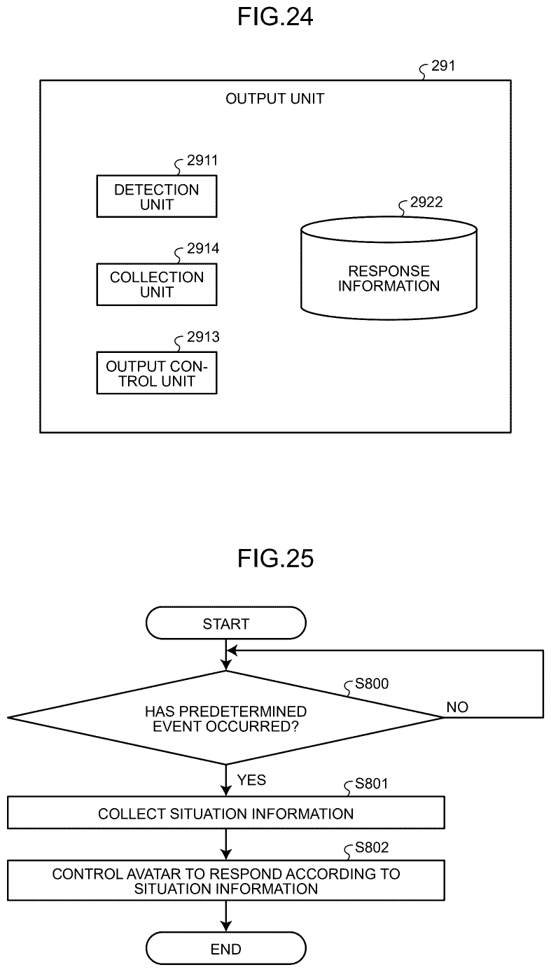

The output unit 291 will be described in detail. FIG. 24 is a diagram schematically illustrating a functional configuration of the output unit 291. As shown in FIG. 24, the output unit 291 includes the detection unit 2911, a collection unit 2914, and the output control unit 2913. The output unit 291 also stores the response information 2922.

The detection unit 2911 detects occurrence of a predetermined event. The detection unit 2911 detects users viewing artwork in cultural facilities such as art museums and galleries. The detection unit 2911 also detects users playing games using various game devices or board games. Furthermore, the detection unit 2911 detects events such as foreign travelers speaking to the avatar 100. The output control unit 2913 controls the avatar 100, which is controlled by the control device 200 equipped with a text generation model, to perform actions according to an event detected by the detection unit 2911.

The collection unit 2914 collects situation information indicating a situation of the user 10. The output control unit 2913 controls the avatar 100 to output information according to the situation information. For example, the collection unit 2914 collects information indicating an artwork being viewed by the user 10 and a situation of the user 10 (such as voice or image) when the user 10 is viewing artwork. The collection unit 2914 also collects information indicating a situation of the user 10 (such as voice, image of user 10) when being spoken to by the user 10 of a foreign traveler. The collection unit 2914 collects situation information related to user's emotions. This allows the control device 200 to recognize emotions of the user 10 from his/her voice, images, and the like.

The output control unit 2913 controls the avatar 100 to perform actions according to situation information collected by the collection unit 2914. For example, the output control unit 2913 controls the avatar 100 to read interest or impression during viewing from emotions of the user 10 viewing artwork and to provide explanations or suggestions for related works according to these. More specifically, the output control unit 2913 controls the avatar 100 to provide explanations according to the user's interest in the artwork or to suggest related works and the like that the user is likely to be interested in. In other words, in this case, the output control unit 2913 controls the avatar 100 to behave as a curator that is personalized for the user 10.

The output control unit 2913 also controls the avatar 100 to provide advice to the user 10 according to emotions of the user 10 playing a game and the game situation. For example, the output control unit 2913 controls the avatar 100 to provide appropriate gameplay or advice according to emotions of the user 10. In other words, in this case, the output control unit 2913 controls the avatar 100 to respond so that the user 10 can enjoy the game together with the avatar 100.

The output control unit 2913 may also control the avatar 100 to provide support such as language translation to foreign travelers during sightseeing, while being attentive to their emotions. Note that travelers are not limited to foreigners. For example, the output control unit 2913 controls the avatar 100 to resolve anxieties or problems of travelers. More specifically, the output control unit 2913 controls the avatar 100 to listen to anxieties or problems of foreign tourists and to provide solutions to the anxieties or problems in a language of the foreign traveler. In another example, the output control unit 2913 controls the avatar 100 to support foreign travelers by translating conversations between the foreign travelers and local people.

FIG. 25 is a diagram schematically illustrating an example of an operation flow by the output unit 291. The output unit 291 determines whether occurrence of a predetermined event has been detected (step S800). If occurrence of a predetermined event has not been detected (step S800; No), the output unit 291 waits until occurrence of a predetermined event is detected.

On the other hand, if occurrence of a predetermined event has been detected (step S800; Yes), the output unit 291 collects situation information indicating a situation of the user 10 (step S801). Subsequently, the output unit 291 controls the avatar 100, which is controlled by the control device 200 equipped with a text generation model, to respond to the user 10 according to the situation information (step S802), and ends the process.

### Example 8 of Embodiment

Next, Example 8 of the embodiment will be described. Here, the description focuses on the differences from the above embodiments, and descriptions of the same configuration and processing as in the above embodiments are omitted. An example of the system 5 according to this embodiment is schematically illustrated in FIG. 1.

Appearance of an avatar may be human-like, as in the avatar 100 and the avatar 101, or may be a character, as in the avatar 102. For example, the avatars 100 and 101 may act as restaurant staff (such as bartenders), outputting responses to users with actual gestures and engaging in conversation with the users.

Here, each control device is equipped with an output function that recognizes a user's state and outputs responses to each avatar according to the recognized state. For example, each control device can recognize emotions of users visiting restaurants and cause an avatar to output responses (such as customer service) according to the recognized emotions, enabling the avatar to provide human-like customer service.

As shown in FIG. 2, the control device 200 includes the sensor unit 210, the sensor module unit 220, the storage unit 230, the state recognition unit 240, the emotion determination unit 242, the behavior recognition unit 244, the behavior determination unit 246, the memory control unit 248, the behavior control unit 250, the controlled object 252, the communication processing unit 280, and the output unit 291. A data structure of the character data 233 is schematically illustrated in FIG. 3.

An example of an operation flow related to determining a behavior of the avatar 100 is schematically illustrated in FIG. 12.

### (Response Output)

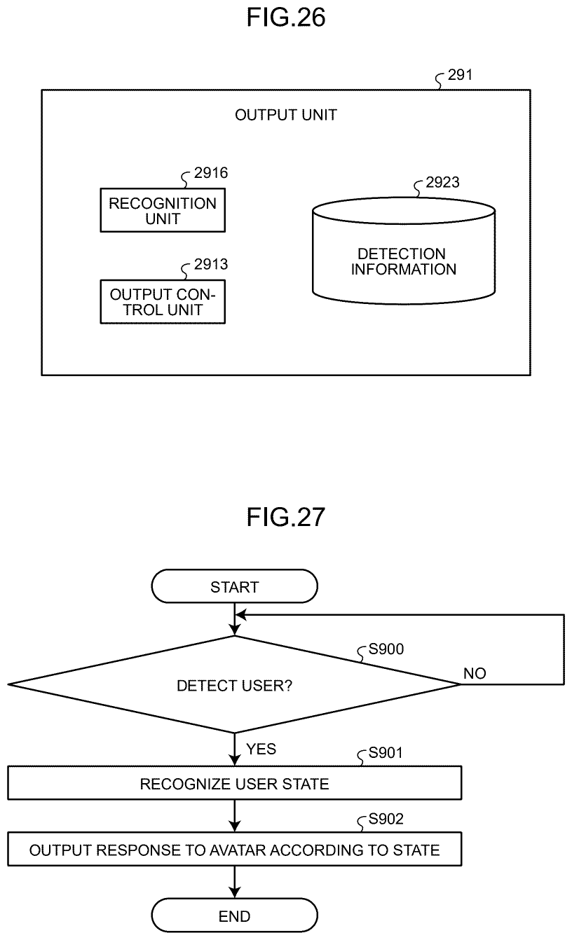

The output unit 291 will be described in detail. FIG. 26 is a diagram schematically illustrating a functional configuration of the output unit 291. As shown in FIG. 26, the output unit 291 includes a recognition unit 2916 and the output control unit 2913. The output unit 291 also stores detection information 2923. The detection information 2923 is implemented by a storage medium such as memory.

For example, the recognition unit 2916 recognizes a state of the user 10 based on detection information detected from the user 10. The output control unit 2913 causes the avatar 100, which is controlled by the control device 200 equipped with a text generation model, to output responses to the user 10 according to the state of the user 10 recognized by the recognition unit 2916. More specifically, the recognition unit 2916 recognizes a state of users visiting restaurants based on detection information. The output control unit 2913 causes the avatar 100 to output concise conversations or explanations to the user 10 in a hurry. On the other hand, for the user 10 who want to talk, the output control unit 2913 causes the avatar 100 to provide customer service or explanations with some small talk. The output control unit 2913 can also cause the avatar 100 to output expressive gestures. This allows the avatar 100 to provide human-like customer service according to a topic and situation with the user 10.

For example, the recognition unit 2916 recognizes emotions of the user 10 based on detection information. The output control unit 2913 causes the avatar 100 to output responses to the user 10 according to the user's emotions. More specifically, if the emotion of the user 10 is “joy,” the output control unit 2913 can cause the avatar 100 to output positive gestures and positive speech corresponding to that emotion, enabling human-like customer service according to emotions of the user 10.

The output control unit 2913 may also cause the avatar 100 to output responses to the user 10 according to a history of detection information stored in the detection information 2923 and a state of the user 10. This allows the avatar 100 to provide customer service such as listening to customers' complaints and giving optimal backchannel responses, as a bar master or café owner would, and to respond to continued complaints based on the history of past complaints based on contents of the past complaints.

For example, the recognition unit 2916 recognizes whether the user 10 is lost on the way to a destination based on detection information. If the user 10 is lost, the output control unit 2913 causes the avatar 100 to output responses related to route guidance. The output control unit 2913 may also cause the avatar 100 to output responses related to route guidance according to detection information. The output control unit 2913 may also cause the avatar 100 to output responses related to route guidance according to emotions of the user 10 based on detection information.

In large hospitals such as university hospitals, the user 10 may get lost on the way to examinations or tests. In such cases, the avatar 100 can recognize whether the user 10 is lost from facial expression or behavior indicated by detection information and guide the lost user 10 to his/her destination. Avatar 100 can also adjust the politeness of guidance and a walking speed according to an age, a medical condition, and emotions of the user 10, enabling careful guidance to the destination for the user 10.

The output control unit 2913 may also cause the avatar 100 to output responses related to proposals for transaction targets according to a state of the user 10. This allows the avatar 100, for example, to recognize background, reasons for moving, economic situation, and preferences for real estate based on detection information of the user 10 considering moving, and to introduce suitable properties. Avatar 100 can also recognize expectations and anxieties associated with moving and provide customer service that is attentive to those emotions (for example, positive gestures or speech when the user is anxious).

For example, the output control unit 2913 may also cause the avatar 100 to output gestures according to a state of the user 10. This allows the avatar 100 to perform reaction gestures according to emotions of the user 10, enabling human-like customer service.

The detection information may be audio, facial expressions, or actions of the user 10 detected by various sensors of the control device 200, or may indicate a situation around the user 10.

The output control unit 2913 may cause the avatar 100 to output responses according to a state of the user 10 as described above, or may cause the avatar 100 to output responses to the user 10 at a timing according to a state of the user 10.

FIG. 27 is a diagram schematically illustrating an example of an operation flow by the output unit 291. The output unit 291 determines whether the user 10, who is a target for the avatar 100 to output responses, has been detected (step S900). If the user 10 has not been detected (step S900; No), the output unit 291 waits until the user 10 is detected.

On the other hand, if the user 10 has been detected (step S900; Yes), the output unit 291 recognizes a state of the user 10 based on detection information detected from the user 10 (step S901). Subsequently, the output unit 291 causes the avatar 100 to output responses to the user 10 according to the state of the user 10 (step S902), and ends the process.

### Example 9 of Embodiment

Next, Example 9 of the embodiment will be described. Here, the description focuses on the differences from the above embodiments, and descriptions of the same configuration and processing as in the above embodiments are omitted. An example of the system 5 according to this embodiment is schematically illustrated in FIG. 1.

Appearance of an avatar may be human-like, as in the avatar 100 and the avatar 101, or may be a character, as in the avatar 102. For example, the avatars 100 and 101 may act as audience members, and based on a content of the user 10's presentation and emotions of the avatars 100 and 101 that change according to the presentation content, output gestures (such as changing facial expressions, laughing, or nodding).

Here, each control device is equipped with an output function that evaluates a state of the user 10 and, according to the evaluation, outputs proposal information regarding suggestions for the state of the user 10 from each avatar to a predetermined output destination. For example, each avatar can make suggestions according to evaluation of a state of the user 10, thereby improving the state of the user 10.

As shown in FIG. 2, the control device 200 includes the sensor unit 210, the sensor module unit 220, the storage unit 230, the state recognition unit 240, the emotion determination unit 242, the behavior recognition unit 244, the behavior determination unit 246, the memory control unit 248, the behavior control unit 250, the controlled object 252, the communication processing unit 280, and the output unit 291. The data structure of the character data 233 is schematically illustrated in FIG. 3.

An example of an operation flow related to determining a behavior of the avatar 100 is schematically illustrated in FIG. 12.

### (Information Output)

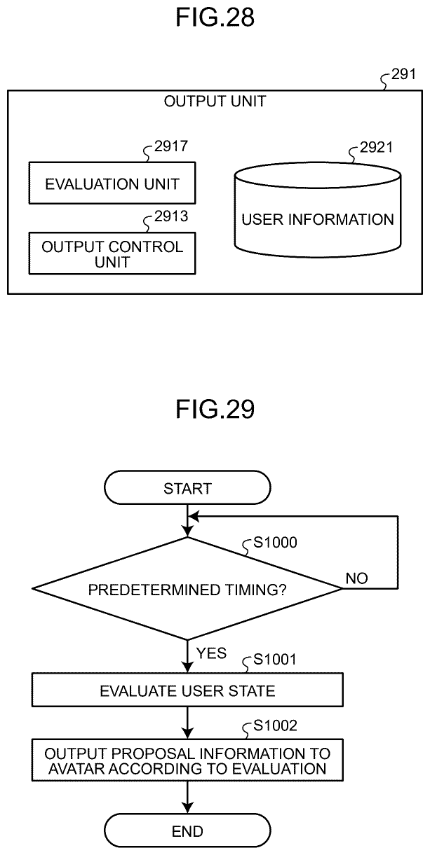

The output unit 291 will be described in detail. FIG. 28 is a diagram schematically illustrating a functional configuration of the output unit 291. As shown in FIG. 28, the output unit 291 includes an evaluation unit 2917 and the output control unit 2913. The output unit 291 also stores the user information 2921.

For example, the evaluation unit 2917 evaluates a state of the user 10 based on user information regarding the user 10 accumulated in the user information 2921. The output control unit 2913 causes the avatar 100, which is controlled by the control device 200 equipped with a text generation model, to output proposal information regarding suggestions for a state of the user 10 to a predetermined output destination according to evaluation by the evaluation unit 2917. More specifically, the evaluation unit 2917 evaluates speech of the user 10 based on user information regarding speech of the user 10. The output control unit 2913 causes the avatar 100 to output proposal information regarding suggestions for the speech of the user 10 according to the evaluation. More specifically, when the user 10 is practicing a presentation, the evaluation unit 2917 analyzes the presentation based on user information related to a content of the presentation, word choice, facial expressions, gestures, and voice tone, and evaluates the presentation (for example, how the public or audience will feel and react). The output control unit 2913 then outputs information on improvements to the presentation in a demonstration format, representing feelings of the public or audience, such as appropriate content, words, facial expressions, gestures, and voice tone. This allows the user 10 to know the public or audience's reaction in advance and prevent risk of decrease in reputation, and also to know how to modify the presentation to improve reputation.

The evaluation unit 2917 may also evaluate a state of the user 10 in an organization based on user information indicating a context of the user 10 belonging to the predetermined organization. The output control unit 2913 causes the avatar 100 to output proposal information regarding suggestions for a state of the user 10 in the organization according to the evaluation. More specifically, the evaluation unit 2917 evaluates motivation, stress, and other states of each the user 10 in the organization based on background, emotions, and needs of each the user 10 belonging to organizations such as companies, volunteer groups, or non-profit organizations. The output control unit 2913 then outputs information on communication improvement in organizations, strategic approaches to achieving organizational goals, educational programs, support for maintaining motivation, and information for relieving stress to each the user 10 via the avatar 100 according to the evaluation. This allows the avatar 100 to respond to emotions and needs of members (the users 10) with diverse backgrounds in an organization, promote diversity and inclusion within the organization, and support understanding and cooperation among the members.

The evaluation unit 2917 may also evaluate a state of the user 10 in a predetermined job based on user information indicating a context of the user 10 performing the predetermined job. The output control unit 2913 causes the avatar 100 to output proposal information regarding suggestions for a state of the user 10 in the job according to the evaluation. More specifically, the evaluation unit 2917 evaluates motivation, stress, and other states of the user 10 in the job based on background, emotions, needs, and stress of the user 10, such as an employee of a company (for example, the user 10 working from home). The output control unit 2913 then outputs advice on emotion and stress management, efficient work methods, appropriate ways to take breaks, and methods for balancing work and private life via the avatar 100 according to the evaluation. This allows the avatar 100 to support improvement in the way the user 10 works in remote work and reduce mental burden.

The evaluation unit 2917 may also evaluate a state of the user 10 regarding career change based on user information indicating a context of the user 10. The output control unit 2913 causes the avatar 100 to output proposal information regarding suggestions for a state of the user 10 regarding career change according to the evaluation. More specifically, the evaluation unit 2917 evaluates motivation, stress, and other states of the user 10 based on the background, emotions, needs, and stress of the user 10 considering a career change. The output control unit 2913 then outputs information on emotional support such as maintaining motivation and relieving stress, as well as methods for promoting self-understanding, skill-up methods for smooth career transition, and networking methods via the avatar 100. This allows the avatar 100 to support appropriate career choices for the user 10.

The evaluation unit 2917 may also evaluate a state of the user 10 regarding career advancement based on user information indicating a context of the user 10. The output control unit 2913 causes the avatar 100 to output proposal information regarding suggestions for a state of the user 10 regarding career advancement according to the evaluation. More specifically, the evaluation unit 2917 evaluates motivation, stress, and other states of the user 10 based on the background, emotions, needs, and stress of the user 10 aiming for career advancement. The output control unit 2913 then outputs information on personalized guidance based on emotional needs and ambitions of the user 10, information on goal setting, progress management, and methods for acquiring appropriate skills to the user 10 via the avatar 100 according to the evaluation. This allows the avatar 100 to provide personalized guidance for individuals aiming for career advancement based on their emotional needs and ambitions.

The evaluation unit 2917 may also evaluate a transaction target (such as a product or service) based on user information indicating evaluation from the user 10 who received the transaction target. The output control unit 2913 causes the avatar 100 to output proposal information regarding suggestions for a transaction target to a provider of the transaction target according to the evaluation. More specifically, the evaluation unit 2917 evaluates emotions and requests of the user 10 regarding a transaction target based on reviews and feedback from the user 10 for the transaction target. The output control unit 2913 then outputs information on improvements to a transaction target to a provider via the avatar 100 according to the evaluation. This allows the avatar 100 to contribute to improving customer satisfaction.

The evaluation unit 2917 may also evaluate administrator managing the user 10 based on emotions of the user 10 derived from user information. The output control unit 2913 causes the avatar 100 to output proposal information regarding suggestions for management of the user 10 to an administrator according to the evaluation. More specifically, the evaluation unit 2917 evaluates motivation, stress, emotions, and other states of the user 10 based on background, emotions, needs, and stress of the user 10. The output control unit 2913 then outputs information on responses and advice for the user 10 to the administrator via the avatar 100 according to the evaluation. This allows the avatar 100 to support administrator's leadership and decision-making by outputting appropriate responses and advice according to emotions of subordinates (the user 10) of the administrator. The avatar 100 can also output information on emotion coaching to an administrator and support self-management.

The user information may be audio, facial expressions, or actions of the user 10 detected by various sensors of the control device 200, or may indicate a situation around the user 10. The user information may also be information acquired from an external server.

FIG. 29 is a diagram schematically illustrating an example of an operation flow by the output unit 291. The output unit 291 determines whether it is a predetermined timing to evaluate a state of the user 10 (step S1000). If it is not a predetermined timing (step S1000; No), the output unit 291 waits until the predetermined timing.

On the other hand, if it is a predetermined timing (step S1000; Yes), the output unit 291 evaluates a state of the user 10 based on user information regarding the user 10 (step S1001). Subsequently, the output unit 291 causes the avatar 100 to output proposal information regarding suggestions for a state of the user 10 to a predetermined output destination according to the evaluation (step S1002), and ends the process.

### Example 10 of Embodiment

Next, Example 10 of the embodiment will be described. Here, the description focuses on the differences from the above embodiments, and descriptions of the same configuration and processing as in the above embodiments are omitted. An example of the system 5 according to this embodiment is schematically illustrated in FIG. 1.

Here, each control device is equipped with an output function that outputs information according to a user's social situation or occurrence of events related to the user via each avatar. For example, each control device can output appropriate information according to a user's situation or occurrence of events related to a user by outputting information related to emotional support for business persons with labor issues via an avatar controlled by the control device.

As shown in FIG. 2, the control device 200 includes the sensor unit 210, the sensor module unit 220, the storage unit 230, the state recognition unit 240, the emotion determination unit 242, the behavior recognition unit 244, the behavior determination unit 246, the memory control unit 248, the behavior control unit 250, the controlled object 252, the communication processing unit 280, and the output unit 291.

The character data 233 included in the storage unit 230 is data associating characters with ages and setting conditions. The character may be a person with characteristics of a specific profession, such as a counselor, doctor, nurse, teacher, lawyer, fortune teller, or insurance salesperson. The character may also be modeled after a real (or formerly real) celebrity. A data structure of the character data 233 is schematically illustrated in FIG. 15.

An example of an operation flow related to determining the behavior of the avatar 100 is schematically illustrated in FIG. 12.

### (Information Output)

The output unit 291 will be described in detail. As shown in FIG. 16, the output unit 291 includes the detection unit 2911, the setting unit 2912, and the output control unit 2913. The output unit 291 also stores the user information 2921 and the response information 2922.

### (Support for Adapting to Circumstances)

Here, the detection unit 2911 detects occurrence of events related to user's circumstances. The output control unit 2913 outputs information according to the detected event. For example, events related to user's circumstances include studying abroad, changing jobs (employment, changing jobs, reemployment), birth of a child, and a child reaching a certain age.

Avatar 100 experiences emotions according to occurrence of events. Specifically, when an event is detected, among the multiple types of emotions prepared in advance as robot's emotions, each of which can be set to a corresponding value, a value of emotion of relief (for example, “relief” in the emotion map described later) is changed. The avatar 100 then outputs according to the emotion value.

This is because when the avatar 100 detects such events, it provides advice and the like related to the user's circumstances. To provide convincing advice, the avatar 100 itself needs to be calm. Note that instead of increasing a relief emotion value, the avatar 100 may reduce all emotion values overall, suppressing emotional expression and providing calm, matter-of-fact advice.

For example, the avatar 100 may output, by speech, text explaining how to adapt to circumstances. This allows the avatar 100 to encourage the user's emotions to change in a positive direction (relief, ease).

The detection unit 2911 can detect events from user's speech. For example, if a user says “I came to Japan to study abroad,” the detection unit 2911 detects that user's circumstances have changed due to studying abroad. For example, if a user says “I'm thinking of changing jobs to an IT company,” the detection unit 2911 detects that the user is trying to change jobs. If a user says “My athletic son became an elementary school student,” the detection unit 2911 detects that the user's child has reached a certain age.

The output control unit 2913 also outputs information for adapting to changed circumstances, which is stored in the response information 2922, via the avatar 100. This allows the output unit 291 to support user's adaptation to circumstances in response to events.

The emotion determination unit 242 increases a “relief” emotion value (strengthens the emotion) in response to occurrence of an event. Alternatively, the emotion determination unit 242 may decrease all emotion values overall in response to occurrence of an event. The output control unit 2913 controls an output of the avatar 100 according to an emotion value of the avatar 100. For example, the output control unit 2913 causes the avatar 100 to speak clearly and matter-of-factly.

When the detection unit 2911 detects occurrence of an event, the setting unit 2912 refers to the character data 233 and sets a character of the avatar 100. According to setting by the setting unit 2912, appearance of the avatar 100 displayed on a display device and the like changes.

For example, when the detection unit 2911 detects occurrence of an event related to user's circumstances, the setting unit 2912 sets a character expected to reassure the user. Specifically, when the detection unit 2911 detects occurrence of an event, the setting unit 2912 can set the avatar 100 as a character modeled after a counselor, teacher, or the user's parent. The setting unit 2912 may also set the avatar 100 as a character modeled after a person of the same race as the user when the detection unit 2911 detects occurrence of an event.

The behavior determination unit 246 and the behavior control unit 250 may also change a processing content according to detection of occurrence of an event by the detection unit 2911. That is, the behavior control unit 250 causes the avatar 100 to perform actions according to an event.

For example, when the detection unit 2911 detects an event related to user's circumstances, the behavior control unit 250 causes the avatar 100 to perform actions to reassure a user when information is output via the avatar 100. Specifically, when information is output via the avatar 100, the behavior control unit 250 can cause the avatar 100 to perform gestures (such as raising a hand or bowing) that are performed as greetings in the user's home country.

For example, the setting unit 2912 refers to the character data 233 and sets a character “G” when a user is a “foreign student,” “Japanese,” and “under 15 years old.”

Below, examples of operations of the output unit 291 will be described along with examples of events.

### (Event: Studying Abroad)

For example, when the detection unit 2911 detects that a user has studied abroad as an event, the output control unit 2913 outputs information on how to adapt to culture and environment of a new place of residence via the avatar 100. The output control unit 2913 also outputs, as methods for coping with challenges and difficulties during studying abroad, information such as efficient language learning methods in a study abroad destination via the avatar 100. Note that outputting via the avatar 100 includes, for example, having the avatar 100 speak.

### (Event: Job Change)

For example, when the detection unit 2911 detects that a user is trying to change jobs, get a job, or be reemployed as an event, the output control unit 2913 outputs vocational training, skill-up methods, and specific curricula according to the user's individual needs and goals for the new job via the avatar 100.

Furthermore, the emotion determination unit 242 can change emotion of the avatar 100 to a positive emotion and have the avatar 100 make statements to encourage the user, thereby improving user's motivation.

### (Event: Child)

For example, when the detection unit 2911 detects that a user's child has been born or has reached a certain age as an event, the output control unit 2913 outputs information suggesting the most suitable extracurricular activities for the child via the avatar 100.

The output control unit 2913 causes the avatar 100 to suggest extracurricular activities according to the child's interests and abilities. The output control unit 2913 may also accept reports on progress of the child's extracurricular activities and output advice via the avatar 100 according to the progress.

### (Output Example Based on Response Rules)

The output control unit 2913 can control the avatar 100 based on the response rule 231. The response rule 231 stores messages to be output when an event related to the user's circumstances is detected. The message may be stored in the response information 2922 instead of the response rule 231.

For example, the response rule 231 stores a message for users who have studied abroad, such as “First, try attending a welcome party for international students.” The response rule 231 also stores a message for users considering changing jobs to an IT company, such as “There are online programming courses available.” The response rule 231 also stores a message for users whose athletic child has become an elementary school student, such as “How about trying a soccer class?”

An example of an operation flow by the output unit 291 is schematically illustrated in FIG. 17.

### Example 11 of Embodiment

Next, Example 11 of the embodiment will be described. Here, description focuses on differences from the above embodiments, and descriptions of the same configuration and processing as in the above embodiments are omitted. An example of the system 5 according to this embodiment is schematically illustrated in FIG. 1.

Here, each control device is equipped with a language support function that detects a user's language acquisition status and outputs information according to the detected acquisition status via each avatar. For example, each control device can detect user's understanding of his/her native language or a foreign language (such as English) and output language instruction according to a level of understanding, and information on language skills and vocabulary via an avatar, thereby outputting appropriate information to the user according to his/her language acquisition status.

As shown in FIG. 11, the control device 200 includes the sensor unit 210, the sensor module unit 220, the storage unit 230, the state recognition unit 240, the emotion determination unit 242, the behavior recognition unit 244, the behavior determination unit 246, the memory control unit 248, the behavior control unit 250, the controlled object 252, the communication processing unit 280, and a support unit 290. A data structure of the character data 233 is schematically illustrated in FIG. 3.

An example of an operation flow related to determining a behavior of the avatar 100 is schematically illustrated in FIG. 12.

### (Support Unit 290)

The support unit 290 will be described in detail. As shown in FIG. 13, the support unit 290 includes the detection unit 2901 and the output control unit 2902.

The detection unit 2901 detects a user's acquisition status in various languages. The output control unit 2902 controls the control device 200 equipped with a text generation model to output information according to the acquisition status detected by the detection unit 2901 to the user 10 via the avatar 100.

For example, the detection unit 2901 detects a user's acquisition status in a language other than his/her native language (for example, English for Japanese user). The detection unit 2901 also detects an acquisition status for each skill in the language. Such skills include, for example, reading, writing, listening, and speaking.

The output control unit 2902 controls the avatar 100 to output information according to the detected acquisition status. For example, the output control unit 2902 outputs a learning curriculum according to an acquisition status and a user's target level. The output control unit 2902 may also output a learning curriculum for each skill. This enables effective language learning with a curriculum according to an acquisition status and a target level of each the user 10.

The detection unit 2901 also detects a user's acquisition status in his/her native language. The output control unit 2902 controls the avatar 100 to output information according to the detected acquisition status in the native language. For example, the detection unit 2901 detects a language development status of an infant (ages 1 to 3, before entering kindergarten) as the acquisition status. The output control unit 2902 outputs information on communication methods between mother and child according to the language development status. The communication methods include, for example, methods related to songs, picture books, conversation, and the like. This supports normal language development in infants.

The detection unit 2901 also detects an acquisition status of foreign languages in infants and kindergarteners. The detection unit 2901 also detects parents' intentions regarding foreign language acquisition goals for the infants and kindergarteners (such as aiming for native-level proficiency). The output control unit 2902 outputs a learning curriculum for each developmental stage (for example, by age) according to the acquisition status and parents' intentions. This enables provision of optimal programs according to a child's developmental stage and allows learning to proceed while maintaining the child's motivation.

The detection unit 2901 also detects an acquisition status of users (such as adults) who have completed foreign language learning once. The detection unit 2901 also detects intended use of the foreign language (such as for business). The output control unit 2902 outputs information on communication skills using foreign languages, cross-cultural understanding, and learning curricula for foreign language learning according to the acquisition status and intended use.

Avatar 100 can be set as, for example, a popular cram school teacher avatar, a teacher avatar from a user's school, or a teacher character from an anime. When outputting information related to a foreign language, the avatar 100 can be set as an avatar of a person from a country where the foreign language is a native language (for example, an American or British person for English). This makes it easier for a user to accept information output from the avatar 100.

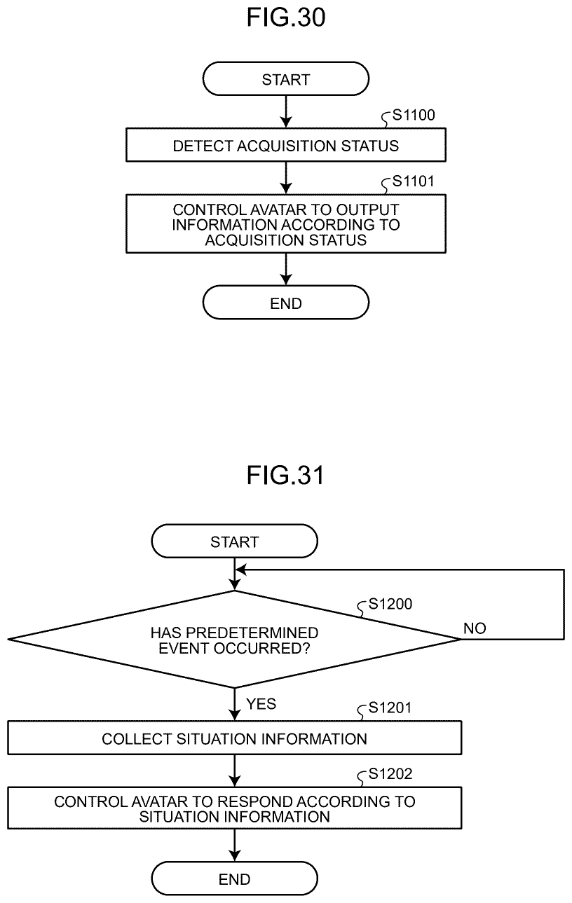

FIG. 30 is a diagram schematically illustrating an example of an operation flow by the support unit 290. First, the support unit 290 detects a user's acquisition status in a language (step S1100). Then, the support unit 290 controls the control device 200 equipped with a text generation model to output information according to the detected acquisition status to the user 10 via the avatar 100 (step S1101), and ends the process.

### Example 12 of Embodiment

Next, Example 12 of the embodiment will be described. Here, description focuses on differences from the above embodiments, and descriptions of the same configuration and processing as in the above embodiments are omitted. An example of the system 5 according to this embodiment is schematically illustrated in FIG. 1.

The appearance of an avatar may be human-like, as in the avatar 100 and the avatar 101, or may be a character, as in the avatar 102. For example, the avatars 100 and 101 may wear coordinated outfits matching those owned by the user 10 and propose outfit coordination to the user 10. The avatars 100 and 101 may also propose outfit coordination including clothes not owned by the user 10.

Here, each the control device 200 is equipped with an event detection function that detects occurrence of a predetermined event and outputs information according to an occurred event. For example, each the control device 200 detects events where the user 10 needs support. In this embodiment, such events include events where the user 10 needs fashion coordination, events where the user 10 seeks relaxation or stress relief, events where the user 10 needs support while traveling, and events where the user 10 needs cooking support. For example, each the control device 200 can recognize emotions of the user 10 and output coordination proposals to the avatar 100 according to the recognized emotions, enabling the avatar 100 to respond like a human. Similarly, each the control device 200 can recognize emotions of the user 10 and output music proposals (to have the avatar 100 sing) according to the recognized emotions, enabling the avatar 100 to respond like a human. Each the control device 200 can also recognize emotions of the user 10 and output sightseeing guidance proposals to the avatar 100 according to the recognized emotions, enabling the avatar 100 to respond like a human. For example, each the control device 200 can also recognize emotions of the user 10 and output recipe proposals to the avatar 100 according to the recognized emotions, enabling the avatar 100 to respond like a human.

As shown in FIG. 2, the control device 200 includes the sensor unit 210, the sensor module unit 220, the storage unit 230, the state recognition unit 240, the emotion determination unit 242, the behavior recognition unit 244, the behavior determination unit 246, the memory control unit 248, the behavior control unit 250, the controlled object 252, the communication processing unit 280, and the output unit 291. A data structure of the character data 233 is schematically illustrated in FIG. 3.

An example of an operation flow related to determining a behavior of the avatar 100 is schematically illustrated in FIG. 12.

### (Event Detection)

The output unit 291 will be described in detail. As shown in FIG. 24, the output unit 291 includes the detection unit 2911, the collection unit 2914, and the output control unit 2913. The output unit 291 also stores the response information 2922.

The detection unit 2911 detects occurrence of a predetermined event. The detection unit 2911 detects events where the user 10 needs fashion coordination. For example, the detection unit 2911 detects gestures such as the user 10 hesitating or choosing an outfit when going out. The detection unit 2911 also detects events where the user 10 seeks relaxation or stress relief. For example, the detection unit 2911 detects these events by detecting speech or specific actions by the user 10 (such as relaxing on a sofa). The detection unit 2911 also detects events where the user 10 seeks sightseeing guidance while traveling. For example, the detection unit 2911 detects such events based on conversations between the user 10 and the avatar 100, and the like. The detection unit 2911 also detects events where the user 10 needs cooking support, and the like. For example, the detection unit 2911 detects such events based on conversations between the user 10 and the avatar 100, and the like.

The collection unit 2914 collects situation information indicating a situation of the user 10. The output control unit 2913 controls the avatar 100 to output information according to situation information. For example, the collection unit 2914 collects information indicating a situation of the user 10 (such as voice or image) when the user 10 is choosing coordination. At this time, the collection unit 2914 may also have the avatar 100 ask the user 10 about a purpose of coordination. The collection unit 2914 also collects information indicating a situation of the user 10 (such as voice or image of the user 10) when an event occurs in which the user 10 seeks relaxation or stress relief. The collection unit 2914 also collects information indicating a situation of the user 10 (such as voice or image of the user 10) when an event occurs in which the user 10 seeks sightseeing guidance. The collection unit 2914 also collects information indicating a situation of the user 10 (such as voice or image of the user 10) when an event occurs in which the user 10 needs cooking support. This allows the control device 200 to recognize emotions of the user 10 from voice, images of the user 10, and the like.

The output control unit 2913 controls the avatar 100 to perform actions according to an occurred event and situation information of the user 10. When an event occurs in which the user 10 needs fashion coordination, the output control unit 2913 controls the avatar 100 to propose fashion items or coordination according to the user 10's body type, face, preferences, mood, season, and situation (for example, the purpose of going out in fashion). For example, the avatar 100 may be in a state of wearing proposed coordination or proposed clothes. The avatar 100 may be in a state of wearing clothes owned by the user 10. That is, the output control unit 2913 controls the avatar 100 to wear the same clothes as the user 10 and, for example, propose “You should wear these clothes today.” The avatar 100 may also be in a state of wearing clothes not owned by the user 10. That is, the output control unit 2913 controls the avatar 100 to wear the same clothes as those not owned by the user 10 (such as clothes of a brand not owned by the user 10) and, for example, propose “I recommend purchasing these clothes.” The output control unit 2913 also controls the avatar 100 to praise the user 10 by saying “It looks good on you” or to encourage the user 10 by saying “Good luck with your interview,” according to the user 10's emotions.

The output control unit 2913 also controls the avatar 100 to output music according to a situation of the user 10 when the user 10 seeks relaxation or stress relief. For example, the output control unit 2913 controls the avatar 100 to output music according to emotions and needs of the user 10. More specifically, if the user 10 seeks relaxation before sleep, the output control unit 2913 controls the avatar 100 to play music that induces sleep and relaxation. The music may be existing music or may be improvised by a music generation AI installed in the control device 200.

The output control unit 2913 also controls the avatar 100 to perform actions according to a situation of the user 10 when an event occurs in which the user 10 seeks sightseeing guidance. For example, the output control unit 2913 controls the avatar 100 to propose recommended spots or activities according to the user 10's interests and situation.

The output control unit 2913 also controls the avatar 100 to perform actions according to a situation of the user 10 when an event occurs in which the user 10 needs cooking support. For example, the output control unit 2913 controls the avatar 100 to propose dishes or recipes according to a situation of the user 10 (cooking skills, available ingredients, available cooking time, etc.) for cooking skills of the user 10. At this time, the output control unit 2913 controls the avatar 100 to propose recipes and the like according to the user 10's preferences and mood.

FIG. 31 is a diagram schematically illustrating an example of an operation flow by the output unit 291. The output unit 291 determines whether occurrence of a predetermined event has been detected (step S1200). If occurrence of a predetermined event has not been detected (step S1200; No), the output unit 291 waits until occurrence of the predetermined event is detected.

On the other hand, if occurrence of a predetermined event has been detected (step S1200; Yes), the output unit 291 collects situation information indicating a situation of the user 10 (step S1201). Subsequently, the output unit 291 controls the avatar 100, which is controlled by the control device 200 equipped with a text generation model, to respond to the user 10 according to an occurred event and situation information (step S1202), and ends the process.

### Example 13 of Embodiment

Next, Example 13 of the embodiment will be described. Here, description focuses on differences from the above embodiments, and descriptions of the same configuration and processing as in the above embodiments are omitted. An example of the system 5 according to this embodiment is schematically illustrated in FIG. 1.

Appearance of an avatar may be human-like, as in the avatar 100 and the avatar 101, or may be a character, as in the avatar 102. For example, the avatars 100 and 101 may act as insurance sales representatives, asking the user 10 questions in a form of hearing to gather necessary information for documents (such as applications) used for insurance enrollment.

Here, each control device is equipped with an output function that recognizes emotions of the user 10 and, according to the emotions of the user 10, outputs information related to insurance that the user 10 is enrolling in from each avatar to the user 10. For example, each avatar can output information according to emotions of the user 10, thereby providing support that is attentive to emotions of the user 10 regarding insurance enrollment.

As shown in FIG. 2, the control device 200 includes the sensor unit 210, the sensor module unit 220, the storage unit 230, the state recognition unit 240, the emotion determination unit 242, the behavior recognition unit 244, the behavior determination unit 246, the memory control unit 248, the behavior control unit 250, the controlled object 252, the communication processing unit 280, and the output unit 291. The data structure of the character data 233 is schematically illustrated in FIG. 15.

An example of an operation flow related to determining a behavior of the avatar 100 is schematically illustrated in FIG. 12.

### (Information Output)

The output unit 291 will be described in detail. As shown in FIG. 26, the output unit 291 includes the recognition unit 2916 and the output control unit 2913. The output unit 291 also stores detection information 2923.

For example, the recognition unit 2916 recognizes emotions of the user 10 based on detection information (for example, detection information accumulated in detection information 2923) detected from the user 10. The output control unit 2913 causes the avatar 100, which is controlled by the control device 200 equipped with a text generation model, to output information related to insurance that the user 10 is enrolling in to the user 10 according to the emotions recognized by the recognition unit 2916. More specifically, the output control unit 2913 causes the avatar 100 to output information to assist in preparation of documents used for insurance enrollment. Even more specifically, the output control unit 2913 causes the avatar 100 to output information related to preparation and completion of insurance contracts and application forms according to the emotions of the user 10. This allows the avatar 100 to reduce hassle of insurance procedures while being attentive to emotions of the user 10.

The output control unit 2913 may also cause the avatar 100 to output information related to insurance proposals for the user 10. More specifically, the output control unit 2913 causes the avatar 100 to output information related to consulting support at a time of insurance enrollment according to emotions of the user 10. This allows the avatar 100 to explain types and contents of insurance in an easy-to-understand manner and propose insurance that meets needs of the user 10 while being attentive to emotions of the user 10, thereby increasing the user's trust.

The output control unit 2913 may also cause the avatar 100 to output responses to questions or consultations from the user 10 regarding insurance. More specifically, the output control unit 2913 provides answers according to emotions of the user 10 to questions or consultations regarding insurance. This allows the avatar 100 to provide support as a consultation desk for insurance while being attentive to emotions of the user 10, thereby increasing trust from the user 10.

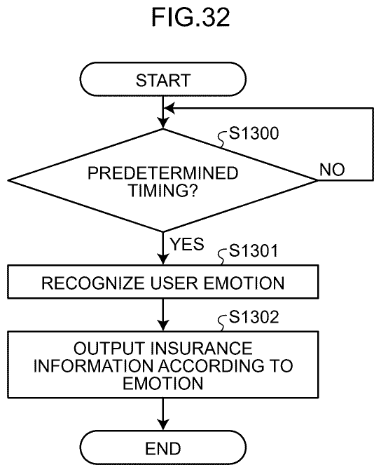

FIG. 32 is a diagram schematically illustrating an example of an operation flow by the output unit 291. The output unit 291 determines whether it is a predetermined timing (for example, a timing when a question or consultation from a user is detected) to output information to the user 10 (step S1300). If it is not the predetermined timing (step S1300; No), the output unit 291 waits until the predetermined timing.

On the other hand, if it is the predetermined timing (step S1300; Yes), the output unit 291 recognizes emotions of the user 10 based on detection information detected from the user 10 (step S1301). Subsequently, the output unit 291 causes the avatar 100 to output information related to insurance that the user 10 is enrolling in to the user 10 according to emotions of the user 10 (step S1302), and ends the process.

### Example 14 of Embodiment

Next, Example 14 of the embodiment will be described. Here, description focuses on differences from the above embodiments, and descriptions of the same configuration and processing as in the above embodiments are omitted. An example of the system 5 according to this embodiment is schematically illustrated in FIG. 1.

Appearance of an avatar may be human-like, as in the avatar 100 and the avatar 101, or may be a character, as in the avatar 102. For example, the avatars 100 and 101 may appear only when a predetermined event (such as a disaster) occurs to advise a user, outputting various individually optimized information such as “Evacuate quickly,” “Put out the fire,” “Evacuate,” “Beware of aftershocks,” and “The evacuation center is here.”

Here, each control device is equipped with an event detection function that detects occurrence of a predetermined event and outputs information according to the occurred event from each avatar. For example, each control device can detect occurrence of a disaster and output appropriate information to a user according to the occurred disaster by outputting emergency response and evacuation guidance information from each avatar.

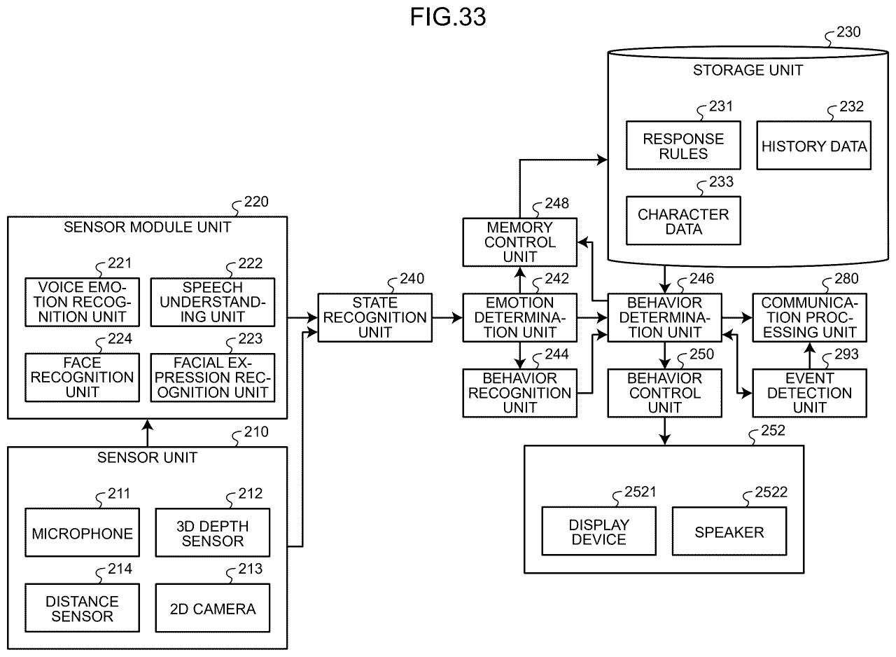

FIG. 33 is a diagram schematically illustrating a functional configuration of the control device 200 for controlling the avatar 100. The control device 200 includes the sensor unit 210, the sensor module unit 220, the storage unit 230, the state recognition unit 240, the emotion determination unit 242, the behavior recognition unit 244, the behavior determination unit 246, the memory control unit 248, the behavior control unit 250, the controlled object 252, the communication processing unit 280, and an event detection unit 293. A data structure of the character data 233 is schematically illustrated in FIG. 15.

The event detection unit 293 implements the above-described output function. Details of the event detection unit 293 will be described later.

An example of an operation flow related to determining a behavior of the avatar 100 is schematically illustrated in FIG. 12.

### (Event Detection)

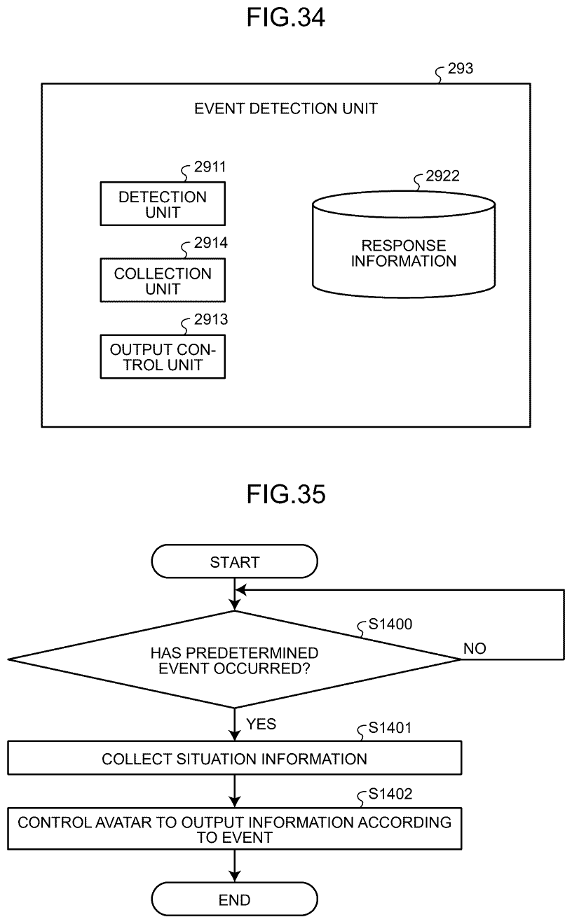

The event detection unit 293 will be described in detail. FIG. 34 is a diagram schematically illustrating a functional configuration of the event detection unit 293. Here, assume that the event detection unit 293 is provided in the control device 200 and causes the avatar 100 to output information according to the detected event.

As shown in FIG. 34, the event detection unit 293 includes the detection unit 2911, a collection unit 2914, and the output control unit 2913. The event detection unit 293 also stores the response information 2922.

Each component of the event detection unit 293 is realized by a CPU operating based on a program. For example, functions of these components can be implemented as CPU operations by basic software (OS) and programs running on the OS.

The detection unit 2911 detects occurrence of a predetermined event. The output control unit 2913 controls the avatar 100, which is controlled by the control device 200 equipped with a text generation model, to output information according to the event detected by the detection unit 2911 to the user 10.

The collection unit 2914 collects situation information indicating a situation of the user 10. The output control unit 2913 controls the avatar 100 to output information according to the situation information. For example, when an event occurs, the collection unit 2914 collects information indicating a situation at a site where the user 10 is present (such as the user's voice). This allows the avatar 100 to understand a situation at a site where the user 10 is present by recognizing emotions of the user 10 from voice of the user 10 and the like, and to provide appropriate instructions for the occurred event through gesture control and the like.

The detection unit 2911 may also detect occurrence of a disaster. The output control unit 2913 controls the avatar 100 to output information according to the occurred disaster.

The output control unit 2913 also controls the avatar 100 to output information related to coping with an occurred event, which is stored in the response information 2922. This allows the avatar 100 to support emergency response and evacuation guidance during disasters.

The detection unit 2911 may detect events such as earthquakes or fires from various sensor information. The detection unit 2911 may also communicate with an external server to detect events such as torrential rain, tornadoes, or typhoons. The detection unit 2911 may also analyze surrounding audio and detect occurrence of events from user speech or audio from television and the like.

FIG. 35 is a diagram schematically illustrating an example of an operation flow by the event detection unit 293. The event detection unit 293 determines whether occurrence of a predetermined event has been detected (step S1400). If occurrence of a predetermined event has not been detected (step S1400; No), the event detection unit 293 waits until occurrence of a predetermined event is detected.

On the other hand, if occurrence of a predetermined event has been detected (step S1400; Yes), the event detection unit 293 collects situation information indicating a situation of the user 10 (step S1401). Subsequently, the event detection unit 293 controls the avatar 100, which is controlled by the control device 200 equipped with a text generation model, to output information according to the occurred event and situation information to the user 10 (step S1402), and ends the process.

### Example 15 of Embodiment

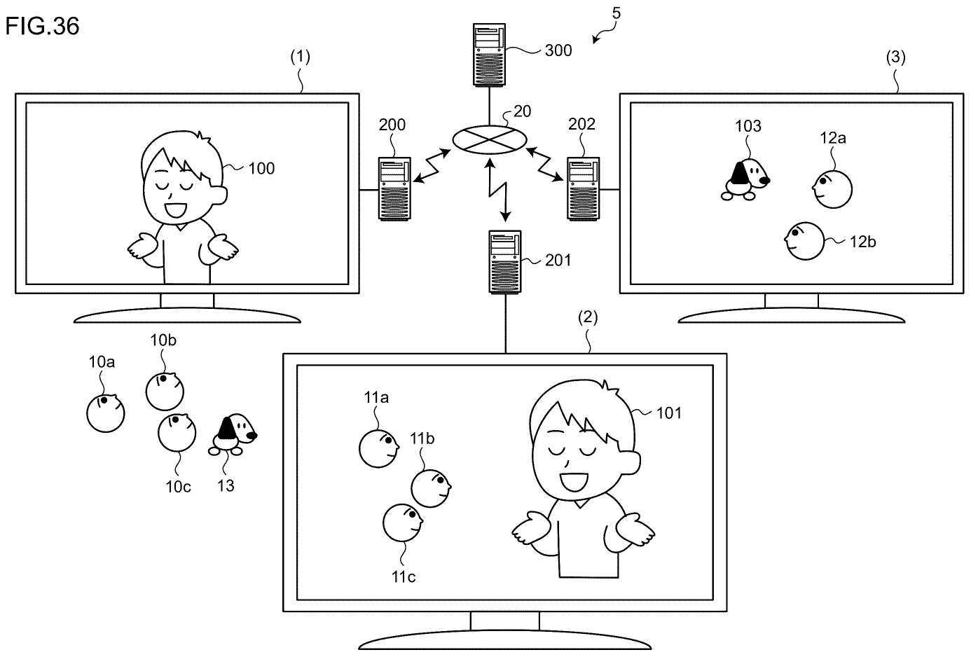

Next, Example 15 of the embodiment will be described. Here, description focuses on differences from the above embodiments, and descriptions of the same configuration and processing as in the above embodiments are omitted. FIG. 36 is a diagram schematically illustrating an example of the system 5 according to the fifteenth embodiment.

The users 10a, 10b, and 10c are users who communicate with the avatar 100. In the description of this embodiment, the users 10a, 10b, and 10c may be collectively referred to as the users 10.

As shown in (3) of FIG. 36, an avatar 103 can also be displayed as a character rather than a person, and communicates with the user 12 as that character. Note that the avatar 103 displayed as such a character may have a form modeled after a pet 13, as described later.

Here, the users 10a, 10b, and 10c constitute a family. In other words, the users 10a, 10b, and 10c are family members. The user 10 also keeps the pet 13. In FIG. 36, the pet 13 is shown as a dog, but this is merely an example and not limiting; for example, the pet 13 may be a cat, rabbit, hamster, or other type of animal.

Note that, as described later, the avatar 100 may provide advice information regarding the pet 13 to the user 10, and in this case, the user 10 does not have to be a family member. Also, as described later, the avatar 100 may provide advice information regarding family to the user 10, and in this case, the user 10 does not have to keep the pet 13.

Appearance of an avatar may be human-like, as in the avatar 100 and the avatar 101, or may have a form modeled after the pet 13 kept by the user 10, as in the avatar 103. By having the avatar 103 with an appearance modeled after the pet 13, it is considered that users will more readily accept the provided advice information, and especially children will find it more familiar.

The server 300 stores user reaction information regarding user's reaction to the avatar 100 received from the control device 200. The server 300 also receives and stores user reaction information not only from the control device 200 that controls the avatar 100, but also from the control devices 201 and 202 that control the avatar 101 and the avatar 103, respectively. The server 300 analyzes user reaction information from the control devices 200, 201, and 202 and updates response rules.

The control device 200 also has a function to recognize a state (behavior and emotion) of the pet 13. The control device 200 recognizes a behavior of the pet 13 and the like by analyzing a face image of the pet 13 acquired by a camera function and voice of the pet 13 acquired by a microphone function. The control device 200 determines a behavior to be executed by the avatar 100 based on the recognized behavior of the pet 13.

The control device 200 according to this embodiment can provide advice information regarding the pet 13 via the avatar 100. For example, the control device 200 recognizes a state of the pet 13. The state of the pet 13 includes behavior and emotions of the pet 13. The control device 200 provides advice information regarding the pet 13 according to a recognized state of the pet 13.

As an example, when the control device 200 recognizes that the pet 13 is showing behavior (appearance) or emotions indicating that it wants to play with the user 10, the control device 200 controls the avatar 100 to perform an action to start a conversation with the user 10. Specifically, the avatar 100 makes a statement such as “I have some advice about your pet,” indicating that advice information will be provided. Note that “pet” in the statement may be a name of the pet 13.

Next, the control device 200 generates advice information regarding the pet 13 based on a recognized state of the pet 13. The advice information may include information about relationship between the pet 13 and the user 10, information about care of the pet 13, and so on, but is not limited to these. Here, the avatar 100 provides advice information appropriate to a state of the pet 13, such as “Your pet wants to play. If you play with it, you'll become closer.”

In this way, in this embodiment, the control device 200 recognizes a state of the pet 13 and causes the avatar 100 to perform an action corresponding to the recognized state of the pet 13, thereby providing appropriate advice regarding the pet 13 to the user 10. That is, according to the control device 200 of this embodiment, the avatar 100 can perform appropriate actions for the user 10. By deepening understanding of a state (feelings and behavior) of the pet 13 and providing advice, the control device 200 can support the user 10 (the pet owner) in building a better relationship with the pet 13.

In addition, the control device 200 according to this embodiment can provide advice information regarding family via the avatar 100. For example, the control device 200 recognizes a state of the user 10, who constitutes the family. A state of the user 10 includes behavior and emotions of the user 10. The control device 200 provides advice information regarding family according to a recognized state of the user 10.

As an example, when the control device 200 recognizes that the user 10a is showing behavior (appearance) or emotions indicating loneliness, the avatar 100 performs an action to start a conversation with the users 10b and 10c, who are family members other than the user 10a. Specifically, the avatar 100 makes a statement such as “I have some advice about the user 10a,” indicating that advice information will be provided. Note that “the user 10a” in the statement may be a name of the user 10a.

Next, the control device 200 generates advice information regarding the user 10a based on a recognized state of the user 10a. The advice information may include information about family communication, but is not limited to this. Here, the avatar 100 provides advice information appropriate to a state of the user 10a, such as “The user 10a is feeling lonely. Try talking to them and communicating.” to the users 10b and 10c.

In this way, in this embodiment, the control device 200 recognizes a state of a family member (for example, the user 10a) and causes the avatar 100 to perform an action corresponding to the recognized state of the user 10 (the user 10a), thereby providing appropriate advice regarding the family to users other than the recognized user (for example, the users 10b and 10c). That is, according to the control device 200 of this embodiment, the avatar 100 can perform appropriate actions for the user 10. By appropriately conveying a state (opinions and emotions) of family and providing advice, the control device 200 can facilitate communication within the family.

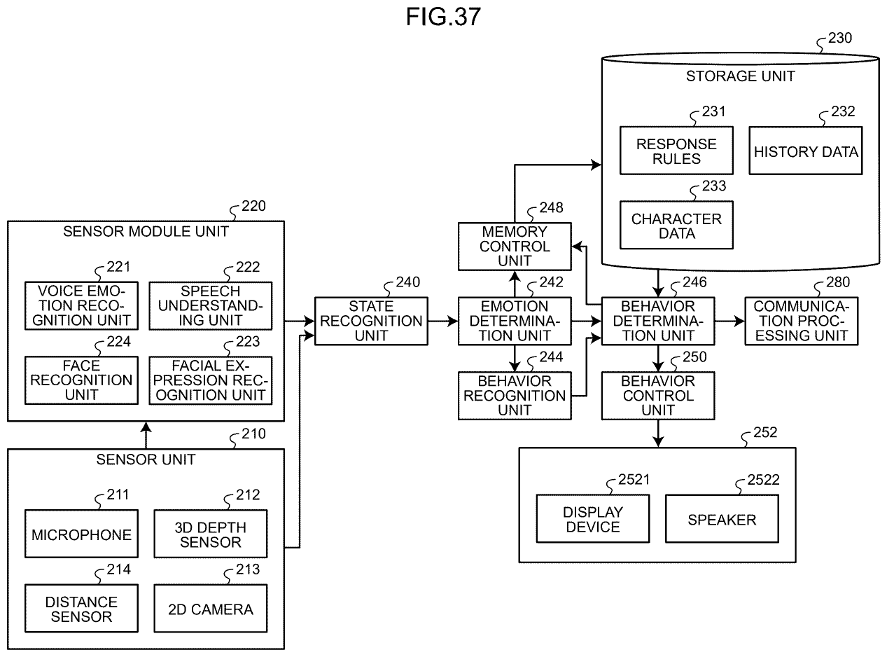

FIG. 37 is a diagram schematically illustrating a functional configuration of the control device 200 for controlling the avatar 100. The control device 200 includes a control unit having the sensor unit 210, the sensor module unit 220, a storage unit (memory unit) 230, the state recognition unit 240, the emotion determination unit 242, the behavior recognition unit 244, the behavior determination unit 246, the memory control unit 248, the behavior control unit 250, the controlled object 252, and the communication processing unit 280. A data structure of the character data 233 is schematically illustrated in FIG. 3.

The controlled object 252 includes the display device 2521 and the speaker 2522, among others. The display device 2521 displays the avatar 100 itself and images related to the avatar 100. The speaker 2522 outputs sounds related to conversation and actions of the avatar 100. Note that posture, gestures, and facial expressions of the avatar 100 are examples of attitudes of the avatar 100.

The history data 232 may include user information for each of a plurality of the users 10 associated with identification information of the users 10. The user information includes information indicating characteristics of the user 10, such as personality, interests, concerns, and preferences. Such user information may be estimated from a behavior history of the user 10 or registered by the user 10 him/herself. The history data 232 may also include past emotion values and a behavior history of the pet 13. This emotion and behavior history may be recorded for each the pet 13 by associating it with identification information of the pet 13. At least part of the storage unit 230 is implemented by a storage medium such as memory. The person DB, which stores face images and attribute information of the user 10, and the pet DB, which stores face images and attribute information of the pet 13, may also be included.

The voice emotion recognition unit 221 of the sensor module unit 220 analyzes voice of the user 10 detected by the microphone 211 and recognizes emotion of the user 10. The voice emotion recognition unit 221 also analyzes voice (barking) of the pet 13 detected by the microphone 211 and recognizes emotion of the pet 13. For example, the voice emotion recognition unit 221 extracts features such as frequency components of voice and recognizes emotion of the pet 13 based on the extracted features.

The facial expression recognition unit 223 recognizes facial expression and emotion of the user 10 from images of the user 10 captured by the 2D camera 213. The facial expression recognition unit 223 also recognizes facial expression and emotion of the pet 13 from images of the pet 13 captured by the 2D camera 213. For example, the facial expression recognition unit 223 recognizes facial expression and emotion of the pet 13 based on shapes and positional relationship of eyes and a mouth.

The face recognition unit 224 recognizes a face of the user 10. The face recognition unit 224 also recognizes a face of the pet 13. The face recognition unit 224 recognizes the pet 13 by matching a face image of the pet 13 captured by the 2D camera 213 with a face image stored in the pet DB (not shown).

The state recognition unit 240 recognizes a state of the user 10 based on information analyzed by the sensor module unit 220. The state recognition unit 240 also recognizes a state of the pet 13 based on information analyzed by the sensor module unit 220. For example, the state recognition unit 240 generates perceptual information such as “The pet is wagging its tail” or “There is an 80% probability that the pet is not making an angry face.” The state recognition unit 240 performs processing to understand meaning of generated perceptual information. For example, the state recognition unit 240 generates semantic information such as “The pet wants to play.”

The emotion determination unit 242 determines a emotion value indicating emotion of the user 10 based on information analyzed by the sensor module unit 220 and a state of the user 10 recognized by the state recognition unit 240. The emotion determination unit 242 also determines an emotion value indicating emotion of the pet 13 based on information analyzed by the sensor module unit 220 and a state of the pet 13 recognized by the state recognition unit 240. For example, information analyzed by the sensor module unit 220 and a recognized state of the pet 13 are input to a pre-trained neural network to obtain an emotion value indicating emotion of the pet 13.

Here, an emotion value indicating emotion of the user 10 or the pet 13 (hereinafter sometimes referred to as “user, etc.”) is a value indicating positivity or negativity of emotion of a user, etc. For example, if emotion of a user, etc. is a bright emotion accompanied by pleasure or comfort, such as “joy,” “pleasure,” “comfort,” “relief,” “excitement,” “ease,” or “fulfillment,” the value is positive, and the brighter the emotion, the larger the value. If emotion of a user, etc. is an unpleasant emotion, such as “anger,” “sorrow,” “discomfort,” “anxiety,” “sadness,” “worry,” or “emptiness,” the value is negative, and the more unpleasant the emotion, the greater an absolute value of a negative value. If emotion of a user, etc. is none of the above (“normal”), the value is zero. Note that types of emotions for the user 10 and the pet 13 may be different or the same. The emotion values for the user 10 may also be different from or the same as those for the pet 13.

The emotion determination unit 242 also determines a emotion value indicating emotion of the avatar 100 based on information analyzed by the sensor module unit 220 and a state of a user, etc. recognized by the state recognition unit 240.

Specifically, the emotion determination unit 242 determines an emotion value indicating emotion of the avatar 100 according to a rule for updating an emotion value of the avatar 100, which is defined in association with information analyzed by the sensor module unit 220 and a state of a user, etc. recognized by the state recognition unit 240.

For example, when the state recognition unit 240 recognizes that a user, etc. seems lonely, the emotion determination unit 242 increases a “sorrow” emotion value of the avatar 100. When a state recognition unit 240 recognizes that a user, etc. is smiling, the emotion determination unit 242 increases a “joy” emotion value of the avatar 100.

The behavior recognition unit 244 recognizes behavior of a user, etc. based on information analyzed by the sensor module unit 220 and a state of the user, etc. recognized by the state recognition unit 240. For example, information analyzed by the sensor module unit 220 and the recognized state of the user, etc. are input to a pre-trained neural network to obtain probabilities of each of a plurality of predefined behavior categories (for example, “laughing,” “angry,” “asking a question,” “sad”), and a behavior category with the highest probability is recognized as a behavior of the user, etc.

The behavior determination unit 246 determines a behavior corresponding to a behavior of a user, etc. recognized by the behavior recognition unit 244, based on a current emotion value of the user, etc. determined by the emotion determination unit 242, the history data 232 of past emotion values determined by the emotion determination unit 242 before a current emotion value of the user, etc. is determined, and an emotion value of the avatar 100. In this embodiment, the behavior determination unit 246 uses the most recent emotion value included in the history data 232 as the past emotion value of the user, etc., but the disclosed technology is not limited to this aspect. For example, the behavior determination unit 246 may use a plurality of most recent emotion values as past emotion values of a user, etc., or may use an emotion value from a unit period such as one day ago. The behavior determination unit 246 may also determine a behavior corresponding to a behavior of a user, etc. by further considering a history of past emotion values of the avatar 100 in addition to a current emotion value of the avatar 100. A behavior determined by the behavior determination unit 246 includes gestures performed by the avatar 100 or speech content of the avatar 100.

The behavior determination unit 246 according to this embodiment determines a behavior of the avatar 100 as a behavior corresponding to a behavior of the user, etc., based on a combination of past and current emotion values of a user, etc., an emotion value of the avatar 100, a behavior of the user, etc., and the response rule 231. For example, when a past emotion value of a user, etc. is positive and a current emotion value is negative, the behavior determination unit 246 determines a behavior for changing an emotion value of the user, etc. to a more positive value as a behavior corresponding to the behavior of the user, etc.

The response rule 231 may include rules for determining a behavior of the avatar 100 based on emotions of the pet 13, emotions of the avatar 100, and a behavior of the pet 13, as well as the user 10. For example, the response rule 231 may define a behavior of the avatar 100 according to a combination of past and current emotion values of the pet 13, an emotion value of the avatar 100, and a behavior of the pet 13.

For example, the response rule 231 defines a behavior of the avatar 100 corresponding to behavior patterns such as when a state of the pet 13 requires advice to the user 10 or when a reaction is received from the user 10 to provided advice information. As an example, based on the response rule 231, when the behavior determination unit 246 recognizes a behavior or emotion of the pet 13 as a state of the pet 13, it determines as a behavior of the avatar 100 an action to provide advice information regarding the pet 13 to the user 10 according to the state of the pet 13.

Similarly, the response rule 231 defines behaviors of the avatar 100 corresponding to behavior patterns such as when a state of the user 10a, who constitutes the family, requires advice to the users 10b and 10c other than the user 10a, or when a reaction is received from the user 10 to provided advice information. As an example, based on the response rule 231, when the behavior determination unit 246 recognizes a behavior or emotion of the user 10a as a state of the user 10a, it determines, as a behavior of the avatar 100, an action to provide advice information regarding the family to the users 10b and 10c according to the state of the user 10a.

The memory control unit 248 determines whether to store data including a behavior of a user, etc. in the history data 232 based on a predetermined intensity of a behavior determined by the behavior determination unit 246 and an emotion value of the avatar 100 determined by the emotion determination unit 242.

Specifically, when a total value of intensities, which is a sum of a total of emotion values for each of the multiple emotion categories of the avatar 100, a predetermined intensity for gestures included in a behavior determined by the behavior determination unit 246, and a predetermined intensity for speech content included in a behavior determined by the behavior determination unit 246, is equal to or greater than a threshold, it is determined that data including a behavior of a user, etc. is to be stored in the history data 232.

When the memory control unit 248 determines to store data including a behavior of a user, etc. in the history data 232, it stores the behavior determined by the behavior determination unit 246, information analyzed by the sensor module unit 220 from the present time to a certain period in the past (for example, all kinds of surrounding information such as audio, images, odors, etc.), and a state of a user, etc. recognized by the state recognition unit 240 (for example, the facial expression and emotion of the user 10, the facial expression and emotion of the pet 13, etc.) in the history data 232.

As described above, in the control device 200 according to this embodiment, the state recognition unit 240 and the behavior recognition unit 244 recognize states (behavior and emotion) of the users 10 and pets 13 constituting the family. The behavior determination unit 246 determines a behavior of the avatar 100 corresponding to recognized states of the users 10 and pets 13. The behavior control unit 250 controls the avatar 100 based on the determined behavior of the avatar 100 (controls the controlled object 252).

Specifically, when the behavior control unit 250 recognizes a behavior of the pet 13 as a state of the pet 13, it determines as a behavior of the avatar 100 an action to provide advice information regarding the pet 13 according to the behavior of the pet 13, and controls the avatar 100 (controls the controlled object 252).

Note that the advice information may include information regarding relationship between the pet 13 and the user 10 (hereinafter sometimes referred to as “relationship information”) and information regarding care of the pet 13 (hereinafter sometimes referred to as “care information”). Relationship information may include information for improving or maintaining a good relationship between the pet 13 and the user 10, but is not limited to these examples. The care information may include information on how to deal with emotional concerns regarding care of the pet 13 and information on daily care of the pet 13, but is not limited to these examples. The advice information may include both relationship information and care information, or either one. That is, the advice information may include at least one of relationship information and care information. The advice information is not limited to relationship information and care information and may include other information such as characteristic information of the pet 13.

For example, when the behavior control unit 250 recognizes behavior (appearance) indicating that the pet 13 wants to play with the user 10, such as wagging its tail or making a sweet sound, it controls the avatar 100 to perform an action to start a conversation with the user 10. Specifically, the avatar 100 makes a statement such as “I have some advice about your pet,” indicating that advice information will be provided.

Next, the behavior control unit 250 generates advice information regarding the pet 13 based on a recognized behavior of the pet 13 and causes the avatar 100 to provide the generated advice information by speech. For example, the behavior control unit 250 causes the avatar 100 to provide advice information appropriate to a state of the pet 13 (here, relationship information), such as “Your pet wants to play. If you play with it, you'll become closer.”

Note that, in the above, the behavior control unit 250 recognizes a behavior of the pet 13 as a state of the pet 13, but it may also recognize emotion of the pet 13. For example, when the behavior control unit 250 recognizes that the pet 13 is barking or baring its teeth and that emotion of the pet 13 is “discomfort” or “anger,” it generates advice information regarding the pet 13 based on the recognized emotion of the pet 13 and causes the avatar 100 to provide the generated advice information by speech. For example, the behavior control unit 250 causes the avatar 100 to provide advice information appropriate to a state of the pet 13 (here, care information), such as “Your pet seems uncomfortable. It will be happy if you feed it,” or “Your pet seems uncomfortable. It will be happy if you take it for a walk.” Since an emotion value of the avatar 100 is determined by the emotion determination unit 242, the behavior control unit 250 may also cause the avatar 100 to provide advice information that is attentive to feelings (concerns) of the user 10, such as “Walking is hard, but I recommend it for your health as well.”

In this way, the behavior control unit 250 according to this embodiment can provide appropriate advice regarding the pet 13 to the user 10 via the avatar 100. By deepening understanding of a state (feelings and behavior) of the pet 13 and providing advice, the behavior control unit 250 can support the user 10 (the pet owner) in building a better relationship with the pet 13.

When the behavior control unit 250 recognizes a state of the user 10 constituting a family, it determines as a behavior of the avatar 100 an action to provide advice information regarding the family according to the recognized state of the user 10, and controls the avatar 100 (controls the controlled object 252).

Specifically, when the behavior control unit 250 recognizes a behavior of the user 10 constituting a family as a state of the user 10, it determines as a behavior of the avatar 100 an action to provide advice information regarding the family according to the behavior of the user 10, and controls the controlled object 252.

For example, when the behavior control unit 250 receives speech such as “I'm lonely” or “I'm bored” from the user 10a and recognizes behavior (appearance) indicating that the user 10a is lonely, it controls the avatar 100 to perform an action to start a conversation with the users 10b and 10c, who are family members other than the user 10a. Specifically, the avatar 100 makes a statement such as “I have some advice about the user 10a,” indicating that advice information will be provided.

Next, the behavior control unit 250 generates advice information regarding a family based on the recognized behavior of the user 10a and causes the avatar 100 to provide the generated advice information by speech. For example, the behavior control unit 250 causes the avatar 100 to provide advice information appropriate to a state of the user 10a, such as “The user 10a is feeling lonely. Try talking to them and communicating,” or “The user 10a is feeling lonely. How about going out together?” to the users 10b and 10c.

Here, the behavior control unit 250 may generate advice information based on user information indicating characteristics of the user 10. For example, the behavior control unit 250 generates advice information based on at least one of personality, interests, concerns, and preferences of each the user 10 included in the user information. As an example, if the user 10a's interest is “shopping,” the behavior control unit 250 causes the avatar 100 to provide advice information appropriate to the user 10a's interest, such as “The user 10a is feeling lonely. How about going shopping together?”

Note that the user information is not limited to personality, interests, concerns, and preferences of the user 10 described above, and may include other characteristics such as hobbies and tastes.

Note that, in the above, the behavior control unit 250 recognizes a behavior of the user 10 as a state of the user 10, but it may also recognize emotion (opinion) of the user 10. For example, when the behavior control unit 250 receives speech such as “The user 10b gets angry easily” or “The user 10b is noisy” from the user 10a and recognizes that the user 10a's emotion toward the user 10b is “discomfort” or “anxiety,” it generates advice information regarding a family based on the recognized emotion of the user 10 and causes the avatar 100 to provide the generated advice information to the user 10b by speech. For example, the behavior control unit 250 causes the avatar 100 to provide advice information appropriate to a state of the user 10a, such as “What you said seems to have made the user 10a uncomfortable. How about changing the way you say it?” to the user 10b. Since a emotion value of the avatar 100 is determined by the emotion determination unit 242, the behavior control unit 250 may also cause the avatar 100 to provide advice information that is attentive to feelings (concerns) of the user 10b, such as “I understand your anger, but if you change the way you say it, your feelings will be better conveyed.”

In this way, the behavior control unit 250 according to this embodiment can provide appropriate advice regarding a family to the user 10 via the avatar 100. By appropriately conveying a state (opinions and emotions) of a family and providing advice, the behavior control unit 250 can facilitate communication within the family; in other words, it can support deepening mutual understanding within a family.

Note that provision of advice information to the user 10 is not limited to speech using the speaker 2522, which is the controlled object 252, and may also be performed through a display of the avatar 100 on the display device 2521, which is the controlled object 252.

The behavior control unit 250 also changes a content of advice information according to emotion of the avatar 100. For example, the behavior control unit 250 changes a content of advice information to be generated according to an emotion value of the avatar 100. Specifically, when the behavior control unit 250 receives speech indicating a rude attitude from a user, an “anger” or “sorrow” emotion value of the avatar 100 increases, and advice information corresponding to the increased emotion value is generated and provided. Such advice information is less detailed or omits some content compared to advice information provided to users who do not exhibit rude speech or behavior (i.e., users with good speech or behavior).

The behavior control unit 250 also generates and provides advice information corresponding to an increased emotion value when an “anger” or “sorrow” emotion value of the avatar 100 increases due to, for example, loud ambient noise making it impossible to detect user's voice. Such advice information is less detailed or omits some content compared to advice information provided when user's voice can be detected.

When receiving positive speech from the user, such as “Thank you for your advice as always,” a “joy” or “pleasure” emotion value of the avatar 100 increases, and the behavior control unit 250 also generates and provides advice information corresponding to the increased emotion value. This advice information differs in content from the advice information provided to users who have not received voice input indicating a favorable attitude (i.e., users with a normal attitude). For example, the advice information provided to users who have received voice input indicating a favorable attitude will contain more extensive advice proposals or additional advice content compared to advice information provided to users who have not received such voice input.

The behavior control unit 250 may also change a frequency of providing advice information to a user according to an emotion value of the avatar 100. For example, when user's reaction to provided advice information is not poor (specifically, when the user gives a positive response such as “Good job”), and a “joy” emotion value of the avatar 100 increases, the behavior control unit 250 may increase frequency of providing advice information to the user. Conversely, when a user's reaction to provided advice information is poor (specifically, when the user gives a negative response such as “That's not right”), and a “sorrow” emotion value of the avatar 100 increases, the behavior control unit 250 may decrease frequency of providing advice information to the user.

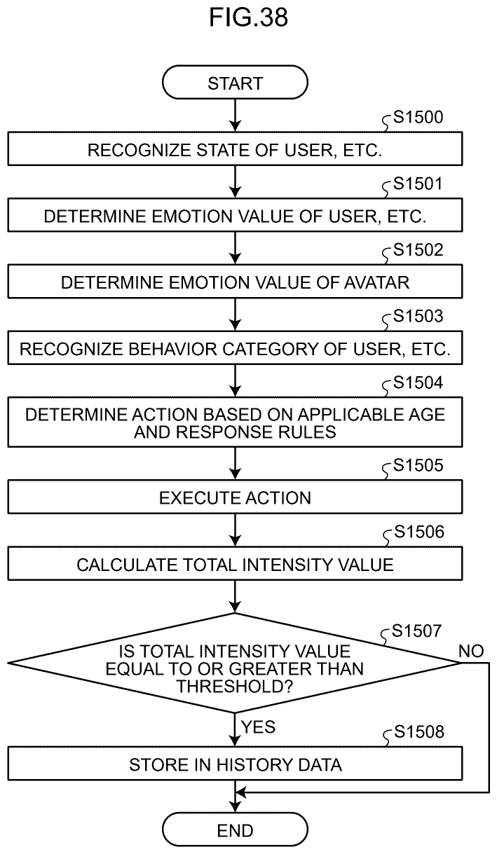

FIG. 38 is a diagram schematically illustrating an example of an operation flow related to determining a behavior of the avatar 100. The operation flow shown in FIG. 38 is repeatedly executed. At this time, it is assumed that information analyzed by the sensor module unit 220 is being input.

First, in step S1500, the state recognition unit 240 recognizes a state of a user, etc. based on information analyzed by the sensor module unit 220. For example, the state recognition unit 240 generates perceptual information such as “Dad is alone” or “There is a 90% probability that dad is not smiling,” and performs processing to understand meaning of the generated perceptual information. For example, the state recognition unit 240 generates semantic information such as “Dad is alone and seems lonely.” The state recognition unit 240 also generates perceptual information such as “The pet is wagging its tail” or “There is an 80% probability that the pet is not making an angry face,” and generates semantic information such as “The pet wants to play.”

In step S1501, the emotion determination unit 242 determines an emotion value indicating emotion of a user, etc. based on information analyzed by the sensor module unit 220 and a state of the user, etc. recognized by the state recognition unit 240.

In step S1502, the emotion determination unit 242 determines an emotion value indicating emotion of the avatar 100 based on information analyzed by the sensor module unit 220 and a state of a user, etc. recognized by the state recognition unit 240. The emotion determination unit 242 adds the determined emotion value of the user, etc. to the history data 232.

In step S1503, the behavior recognition unit 244 recognizes a behavior category of a user, etc. based on information analyzed by the sensor module unit 220 and a state of a user, etc. recognized by the state recognition unit 240.

In step S1504, the behavior determination unit 246 determines a behavior of the avatar 100 based on a target age acquired in step S52 of FIG. 4, a combination of a current emotion value of the user, etc. determined in step S15101 of FIG. 38 and a past emotion value included in the history data 232, an emotion value of the avatar 100, a behavior of the user, etc. recognized by the behavior recognition unit 244, and the response rule 231.

In step S1505, the behavior control unit 250 controls the avatar 100 and the controlled object 252 based on a behavior determined by the behavior determination unit 246. For example, the behavior control unit 250 controls the avatar 100 and the controlled object 252 based on a behavior determined according to a state of the user 10 constituting a family and/or a state of the pet 13.

In step S1506, the memory control unit 248 calculates a total value of intensities based on a predetermined intensity of a behavior determined by the behavior determination unit 246 and an emotion value of the avatar 100 determined by the emotion determination unit 242.

In step S1507, the memory control unit 248 determines whether a total value of intensities is equal to or greater than a threshold. If a total value of intensities is less than a threshold, the process ends without storing data including a behavior of the user, etc. in the history data 232. On the other hand, if a total value of intensities is equal to or greater than the threshold, the process proceeds to step S1508.

In step S1508, a behavior determined by the behavior determination unit 246, information analyzed by the sensor module unit 220 from the present time to a certain period in the past, and a state of a user, etc. recognized by the state recognition unit 240 are stored in the history data 232.

As described above, the control device 200 includes a control unit that recognizes at least one of a state of the user 10 constituting a family and a state of the pet 13 kept by the user 10, determines a behavior of the avatar 100 corresponding to a recognized state, and controls the avatar 100 based on a determined behavior of the avatar 100. As a result, the control device 200 can perform appropriate actions such as providing appropriate advice regarding the family or the pet 13 to the user 10.

The control unit of the control device 200 also determines as a behavior of the avatar 100 an action to provide advice information regarding the pet 13 according to a behavior of the pet 13 when a behavior of the pet 13 is recognized as a state of the pet 13. As a result, the control device 200 can provide appropriate advice information regarding the pet 13 according to the behavior of the pet 13 via the avatar 100.

A control unit of the control device 200 also determines as a behavior of the avatar 100 an action to provide advice information regarding the pet 13 according to emotion of the pet 13 when emotion of the pet 13 is recognized as a state of the pet 13. As a result, the control device 200 can provide appropriate advice information regarding the pet 13 according to emotion of the pet 13 via the avatar 100.

The advice information regarding the pet 13 may include at least one of information regarding relationship between the pet 13 and the user 10 and information regarding care of the pet 13. As a result, the control device 200 can provide, as advice information, for example, information for improving relationship between the pet 13 and the user 10 or information on how to deal with emotional concerns regarding care of the pet 13.

When recognizing a behavior of the user 10 constituting a family as a state of the user 10, a control unit of the control device 200 also determines as a behavior of the avatar 100 an action to provide advice information regarding the family according to the behavior of the user 10. As a result, the control device 200 can provide appropriate advice information regarding the family according to the behavior of the user 10 constituting the family via the avatar 100.

When recognizing emotion of the user 10 constituting a family as a state of the user 10, a control unit of the control device 200 also determines as a behavior of the avatar 100 an action to provide advice information regarding the family according to the emotion of the user 10 constituting the family member. As a result, the control device 200 can provide appropriate advice information regarding the family according to emotion of the user 10 constituting the family member via the avatar 100.

A control unit of the control device 200 also generates advice information based on at least one of personality, interests, concerns, and preferences of each of the users 10 constituting a family. As a result, the control device 200 can provide appropriate advice information regarding a family according to personality and other characteristics of each the user 10.

### Example 16 of Embodiment

Next, Example 16 of the embodiment will be described. Here, description focuses on differences from the above embodiments, and descriptions of the same configuration and processing as in the above embodiments are omitted.

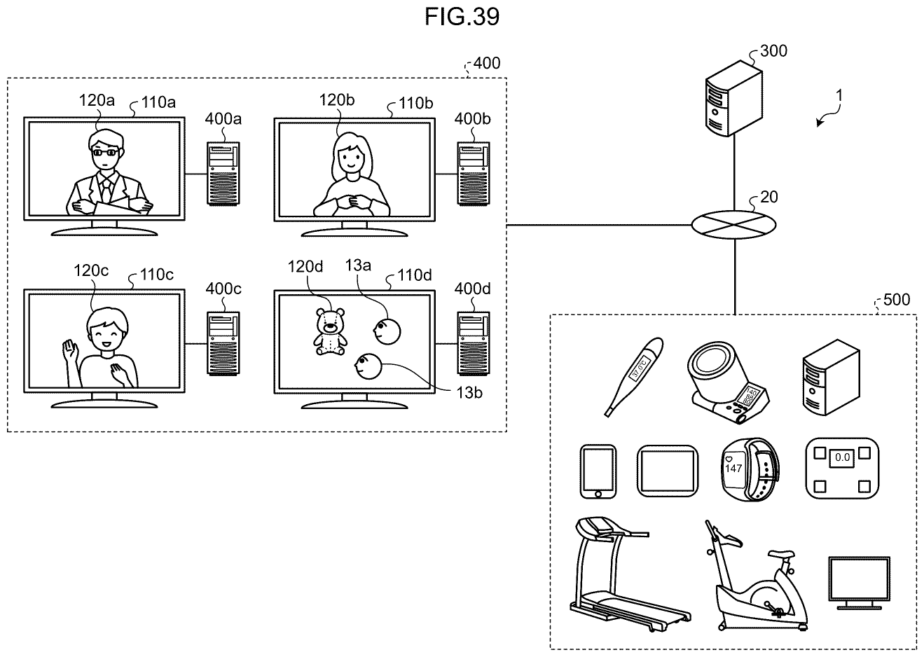

FIG. 39 is a diagram schematically illustrating an example of the control system 1 according to the sixteenth embodiment. As shown in FIG. 39, the control system 1 includes a control device 400 for controlling a plurality of avatars 120, a linked device 500, and the server 300. A plurality of control devices 400a to 400d are managed by respective users. Appearances of the plurality of avatars 120 may be human-like, as in the avatars 120a to 120c, or may be modeled after stuffed animals, as in an avatar 120d. In the description of this embodiment, when the avatars 120a to 120d are not distinguished, they may be simply referred to as the avatars 120. Similarly, when control devices 400a to 400d are not distinguished, they may be simply referred to as the control devices 400. The control device 400 for controlling avatar 120 is wirelessly or wiredly connected to a display device such as a PC monitor or smartphone display, or to a predetermined wearable terminal device related to at least one of augmented reality, virtual reality, or mixed reality. The control device 400 is an example of an electronic device.

As shown in FIG. 39, the avatars 120 controlled by the control device 400 communicate with users in various forms. For example, as shown in screens 110a to 110c of FIG. 39, the avatars 120a to 120c are represented as persons or the like displayed on a display device, and communicate with users via input/output devices provided in the control devices 400a to 400d. As shown in a screen 110d of FIG. 39, the avatar 120d communicates with users 13a and 13b, who are displayed as persons or characters on a display device, in a virtual space. As also shown in the screen 110d of FIG. 39, avatar 120d can be displayed as a character such as a stuffed animal rather than a person, and communicates with the users 13a and 13b as that character.

Appearance of an avatar may be human-like, as in the avatars 120a to 120c, or may be a stuffed animal character, as in the avatar 120d. For example, the avatars 120a to 120c may act as lawyers or counselors, nodding and listening like real lawyers or counselors, thereby providing emotional support and various information to users with divorce issues and the like.

Descriptions of the avatars 120 in this embodiment that are the same as those for the avatar 100 in the above embodiments are omitted. Descriptions of the control device 400 in this embodiment that are the same as those for the control device 200 in the above embodiments are also omitted.

Control device 400 can perform actions in cooperation with a linked device 500. The linked device 500 may be, for example, a thermometer, blood pressure monitor, smartwatch (including those capable of measuring heart rate, etc.), body composition monitor, terminal device (PC, smartphone, tablet, etc.), television, display, training equipment (treadmill, fitness bike, etc.), and so on. These linked devices 500 are communicably connected to the control device 400 via the communication network 20 and exchange information with the control device 400. With this configuration, the linked device 500 can perform its own control, conversations with users, and information provision, etc., according to instructions from the control device 400.

In this disclosure, examples are described in which appropriate information and advice are provided as various actions to users who want to consider various matters. The matters to be considered may include, for example, divorce, legal issues and concerns, procedures at local governments, consumer issues such as consumer troubles, debt (loan problems), and custody issues.

Specifically, the control device 400 is installed in government offices, consultation centers, and the like, and provides various information by conversation or images according to a content and emotions of the user's considerations obtained through conversation with avatar 120. The various matters to be considered by a user are examples of consideration information.

For example, the control device 400 recognizes matters related to divorce through conversation with a user and determines the user's emotions. Control device 400 provides information corresponding to the recognized matters related to divorce to the user, for example, through conversation by the avatar 120 and a screen display on its own display device or a linked device 500 (terminal device). At this time, the avatar 120 may appear as a person modeled after a lawyer, play a role of a conversation partner, and provide appropriate advice while sharing concerns and anxieties according to a conversation content and emotions (user/self). For example, the avatar 120 explains how to file for divorce, examples of property division at divorce, and so on. The avatar 120 may also appear as a person modeled after a fortune teller and play a role of a conversation partner until user's emotions calm down. That is, control device 400 provides at least one of legal information and administrative procedure information related to divorce to a user via the avatar 120 and the like.

The control device 400 also recognizes legal issues and concerns through conversation with a user and determines the user's emotions. The control device 400 provides information corresponding to the recognized legal issues and concerns to the user, for example, through conversation by the avatar 120 and screen display on its own display device or a linked device 500 (terminal device). At this time, the avatar 120 may appear as a person modeled after a lawyer and provide advice on legal issues and concerns according to the conversation content and emotions (user/self). The avatar 120 may also introduce appropriate legal professionals and provide information on legal procedures. That is, the control device 400 provides at least one of information on professionals according to legal issues or concerns and information on legal procedures to a user via the avatar 120 and the like. Note that the user may be not only an individual but also a representative of a company (corporation) or the like.

The control device 400 also recognizes matters related to procedures at local governments (such as city halls) through conversation with a user and determines the user's emotions. The control device 400 provides information corresponding to the recognized matters related to procedures at local governments to the user, for example, through conversation by the avatar 120 and screen display on its own display device or the linked device 500 (namely, terminal device). At this time, the avatar 120 may appear as a person modeled after a local government employee and provide information and advice on procedures at local governments according to the conversation content and emotions (user/self). The avatar 120 may also provide support for preparing necessary documents and proceeding with procedures. For example, if the procedure is a notification of moving in for a change of address, the control device 400 displays the notification of moving in and has the avatar 120 explain sections to be filled out and guide a user to an appropriate counter. That is, the control device 400 provides at least one of information on document preparation and information on how to proceed with procedures to the user via the avatar 120 and the like.

The control device 400 also recognizes consumer issues such as consumer troubles through conversation with a user and determines the user's emotions. The control device 400 provides information corresponding to the recognized consumer issues to the user, for example, through conversation by the avatar 120 and screen display on its own display device or the linked device 500 (terminal device). At this time, the avatar 120 may appear as a person modeled after a consultation desk staff member and provide information and advice on consumer rights, laws, and regulations according to conversation content and emotions (user/self). The avatar 120 may also provide support for consumer troubles and complaint handling. For example, the avatar 120 may introduce consumer organizations or consultation desks according to a case. That is, the control device 400 provides at least one of information on consumer rights, laws, and regulations to a user via the avatar 120 and the like.

The control device 400 also recognizes debt (loan problems) through conversation with a user and determines the user's emotions. The control device 400 provides information corresponding to the recognized debt (loan problems) to the user, for example, through conversation by the avatar 120 and screen display on its own display device or the linked device 500 (terminal device). At this time, the avatar 120 may appear as a person modeled after a consultation desk staff member and provide mental support, such as understanding anxiety and fear and proposing appropriate emotional support and coping methods, according to the conversation content and emotions (user/self). The avatar 120 may also provide information on laws and procedures related to debt and introduce professionals for debt settlement. For example, the avatar 120 may introduce professionals for debt settlement to users with multiple debts. That is, the control device 400 provides at least one of mental support conversation, information on laws and administrative procedures related to debt, and information on professionals according to debt-related information to a user via the avatar 120 and the like.

The control device 400 also recognizes custody issues through conversation with a user and determines the user's emotions. The control device 400 provides information corresponding to recognized custody issues to a user, for example, through conversation by the avatar 120 and screen display on its own display device or the linked device 500 (terminal device). At this time, the avatar 120 may appear as a person modeled after a lawyer and provide support such as understanding the user's emotional expression and providing advice that takes into account the user's position and emotions according to the conversation content and emotions (user/self). The avatar 120 may also provide information on laws and procedures related to custody. For example, the avatar 120 may introduce examples of relationships with children and custody cases. That is, the control device 400 provides at least one of support conversation for a user's position, information on laws related to custody, and information on administrative procedures related to custody to a user via the avatar 120 and the like.

In this disclosure, the control device 400 can provide various information by conversation or images according to content and emotions of user's considerations (consideration information) via the avatar 120 and the like. That is, according to the control device 400 of this disclosure, it is possible to provide information according to user's consideration information and emotions.

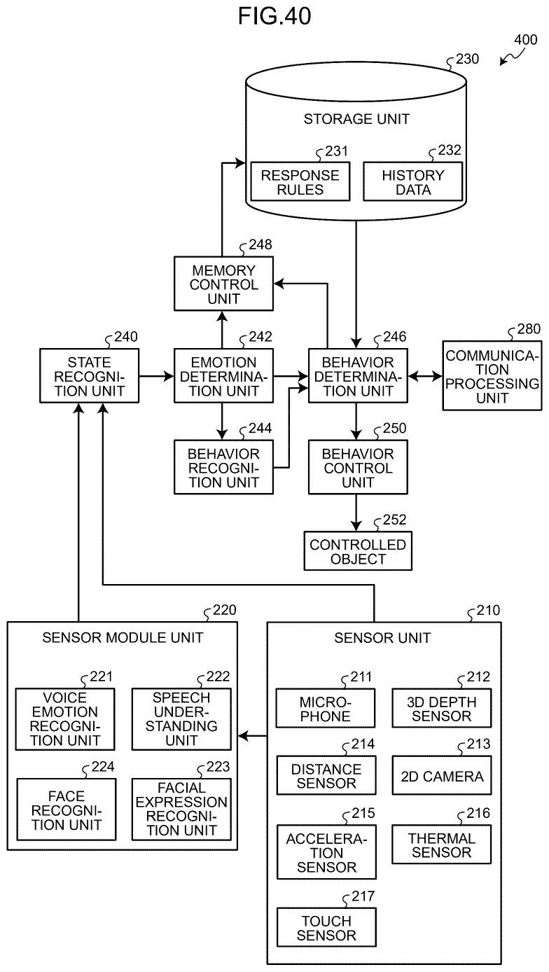

FIG. 40 is a diagram schematically illustrating a functional configuration of the control device 400 for controlling the avatar 120. The control device 400 is composed of a control unit having the sensor unit 210, the sensor module unit 220, the storage unit 230, the state recognition unit 240, the emotion determination unit 242, the behavior recognition unit 244, the behavior determination unit 246, the memory control unit 248, the behavior control unit 250, the controlled object 252, and the communication processing unit 280.

The controlled object 252 includes a display device and a speaker, among others. The display device displays the avatar 120 itself and images related to the avatar 120. The speaker outputs sounds related to conversation and actions of the avatar 120. The display device may also display a conversation content with a user as text. Note that gestures and facial expressions of the avatar 120 are examples of an attitude of the avatar 120.

The sensor unit 210 includes the microphone 211, the 3D depth sensor 212, the 2D camera 213, the distance sensor 214, an acceleration sensor 215, a thermo sensor 216, and a touch sensor 217. The acceleration sensor 215 may be, for example, a gyro sensor and detects an acceleration of the control device 400. The thermo sensor 216 detects an ambient temperature around the control device 400. The touch sensor 217 is a sensor that detects touch operations by a user and may be disposed on a display device connected to the control device 400. The sensor unit 210 may also include a clock, a motor feedback sensor, and others.

The storage unit 230 includes the response rules 231 and the history data 232.

The speech understanding unit 222 analyzes a user's voice detected by the microphone 211 and outputs character information representing user's speech content. For example, the speech understanding unit 222 outputs character information representing contents of various considerations by a user.

The facial expression recognition unit 223 recognizes facial expression and emotion of a user from images of the user captured by the 2D camera 213. For example, the facial expression recognition unit 223 recognizes facial expression and emotion of a user based on a shape and positional relationship of eyes and mouth. For example, the facial expression recognition unit 223 recognizes facial expression and emotion of a user during conversation with a user.

The state recognition unit 240 recognizes a state of a user based on information analyzed by the sensor module unit 220. For example, the state recognition unit 240 mainly performs processing related to perception using analysis results of the sensor module unit 220. For example, the state recognition unit 240 generates perceptual information such as “There is a 90% probability that the user is still anxious” or “There is a 50% probability that the user's thoughts are organized,” and performs processing to understand meaning of the generated perceptual information. For example, the state recognition unit 240 generates semantic information such as “The user is still anxious and seems to want a more simplified explanation.”

The behavior recognition unit 244 recognizes a behavior of a user based on information analyzed by the sensor module unit 220 and a state of the user recognized by the state recognition unit 240. For example, information analyzed by the sensor module unit 220 and a recognized state of a user are input to a pre-trained neural network to obtain probabilities of each of a plurality of predefined behavior categories (for example, “laughing,” “angry,” “asking a question,” “sad”), and a behavior category with the highest probability is recognized as a behavior of the user. For example, the behavior recognition unit 244 recognizes behaviors such as “wondering whether to speak,” “speaking,” or “asking a question” in government offices or consultation centers.

When a combination including at least one of a user's past emotion value and current emotion value, an emotion value of avatar 120, and the user's behavior satisfies conditions of a preset response rule satisfy a pre-set response rule, the behavior determination unit 246 according to this embodiment determines a behavior of avatar 120 associated with the response rule 231. For example, when the user's past emotion value is positive and the current emotion value is negative, the behavior determination unit 246 determines a behavior for changing a user's emotion value to a more positive value as a behavior corresponding to the user's behavior.

For example, the response rule 231 defines a behavior of avatar 120 corresponding to behavior patterns such as when a user is wondering whether to speak, when the user speaks, when the user asks a question, and the like.

The behavior control unit 250 controls the avatar 120 and a controlled object based on a behavior of the avatar 120 determined by the behavior determination unit 246. Specifically, the behavior control unit 250 controls a movement of the avatar 120 and an operation of the controlled object related to the movement of avatar 120 based on a behavior of avatar 120 associated with the response rule 231 determined by the behavior determination unit 246. For example, in a case where the avatar 120 and a user are having a conversation, if the behavior determination unit 246 determines a behavior including speech, the behavior control unit 250 controls avatar 120 to take a predetermined action and causes a speaker included in a controlled object to output voice of the avatar 120. At this time, the behavior control unit 250 may determine a speech speed based on an emotion value of avatar 120. For example, the behavior control unit 250 determines a faster speech speed as an emotion value of the avatar 120 increases. In this way, the behavior control unit 250 determines an execution mode of a behavior determined by the behavior determination unit 246 based on an emotion value determined by the emotion determination unit 242. For example, the behavior control unit 250 controls avatar 120 to say “Are you in trouble?” when a user is wondering whether to speak, “What can I do for you?” when a user speaks, and “You can complete the procedure here” when a user asks a question, thereby guiding the user. When a user's emotion is negative, such as “sorrow,” “discomfort,” “anxiety,” “sadness,” “worry,” or “emptiness,” the behavior control unit 250 controls the avatar 120 to say “It's okay” or “Leave it to me” to ease the user's negative emotions.

The communication processing unit 280 is responsible for communication with the server 300. The communication processing unit 280 can send and receive information with the linked device 500.

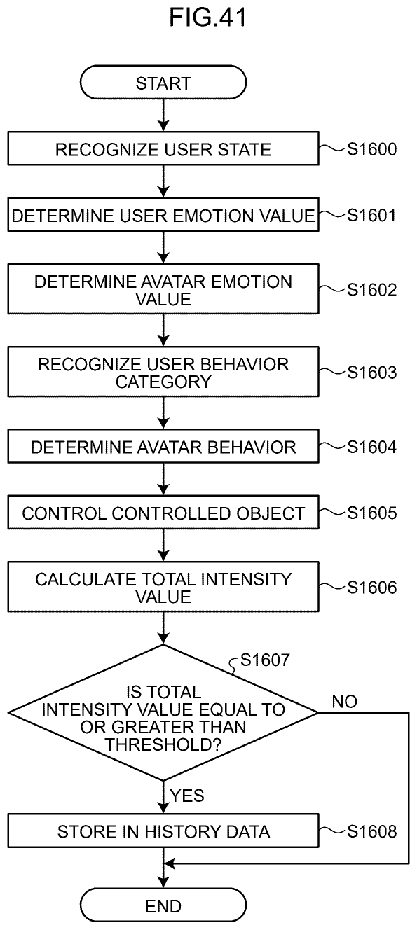

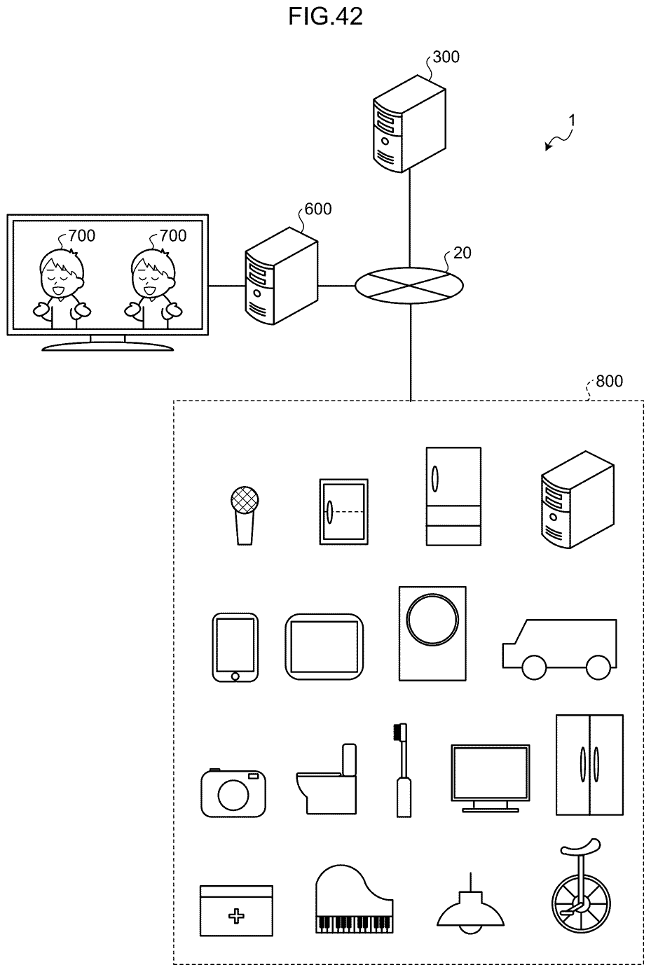

FIG. 41 is a diagram schematically illustrating an example of an operation flow related to determining a behavior of the avatar 120 in the control device 400. The operation flow shown in FIG. 41 is repeatedly executed. At this time, it is assumed that information analyzed by the sensor module unit 220 is being input.

First, in step S1600, the state recognition unit 240 recognizes a state of a user based on information analyzed by the sensor module unit 220. For example, the state recognition unit 240 generates perceptual information such as “There is a 90% probability that the user is still anxious” or “There is a 50% probability that the user's thoughts are organized,” and performs processing to understand meaning of the generated perceptual information. For example, the state recognition unit 240 generates semantic information such as “The user is still anxious and seems to want a more simplified explanation.”

In step S1601, the emotion determination unit 242 determines an emotion value indicating emotion of a user based on information analyzed by the sensor module unit 220 and a state of the user recognized by the state recognition unit 240.

In step S1602, the emotion determination unit 242 determines an emotion value indicating emotion of the avatar 120 based on information analyzed by the sensor module unit 220 and a state of the user recognized by the state recognition unit 240. The emotion determination unit 242 adds the determined emotion value of the user to the history data 232.

In step S1603, the behavior recognition unit 244 recognizes a behavior category of a user based on information analyzed by the sensor module unit 220 and a state of the user recognized by the state recognition unit 240. For example, the behavior recognition unit 244 recognizes behaviors such as “wondering whether to speak,” “speaking,” or “asking a question” in government offices or consultation centers.

In step S1604, the behavior determination unit 246 determines a behavior of avatar 120 associated with the response rule 231 when a combination including at least one of the current emotion value of a user determined in step S1601 and the past emotion value included in the history data 232, an emotion value of avatar 120, and a behavior of the user recognized by the behavior recognition unit 244 satisfies conditions of a preset response rule.

In step S1605, the behavior control unit 250 controls the avatar 120 and the controlled object 252 based on a behavior of the avatar 120 determined by the behavior determination unit 246. For example, the behavior control unit 250 controls the avatar 120 to perform actions such as guiding a user when the user is wondering whether to speak, eliciting the user's requirements, and guiding the user to a counter for answering or processing the user's request.

In step S1606, the memory control unit 248 calculates a total value of intensities based on a predetermined intensity of a behavior determined by the behavior determination unit 246 and an emotion value of the avatar 120 determined by the emotion determination unit 242.

In step S1607, the memory control unit 248 determines whether a total value of intensities is equal to or greater than a threshold. If the total value of the intensities is less than the threshold, the process ends without storing data including a behavior of a user in the history data 232. On the other hand, if the total value of the intensities is equal to or greater than the threshold, the process proceeds to step S1608.

In step S1608, a behavior determined by the behavior determination unit 246, information analyzed by the sensor module unit 220 from the present time to a certain period in the past, and a state of a user recognized by the state recognition unit 240 are stored in the history data 232.

As described above, the control device 400 includes a control unit that recognizes user's consideration information and determines user's emotions, and, based on the determined emotions, conducts a conversation with the user and controls the avatar 120 to provide information corresponding to the recognized consideration information to the user. As a result, the control device 400 can provide information according to the user's consideration information and emotions using the avatar 120.

The consideration information may be information related to divorce, and the control device 400 provides at least one of legal information and administrative procedure information related to divorce to a user. As a result, the control device 400 can provide information related to legal matters regarding divorce to users considering divorce, while being attentive to their emotions, using the avatar 120.

The consideration information may be information related to at least one of legal issues and concerns, and the control device 400 provides at least one of information on professionals according to legal issues or concerns and information on legal procedures to a user. As a result, the control device 400 can provide advice on legal issues and concerns, introduce appropriate legal professionals, and provide information on legal procedures to users with legal issues or concerns, while being attentive to their emotions, using the avatar 120.

The consideration information may be information related to procedures at local governments, and the control device 400 provides at least one of information on document preparation and information on how to proceed with procedures to a user. As a result, the control device 400 can provide information and advice on procedures at local governments, as well as support for preparing necessary documents and proceeding with procedures, to users considering such procedures, while being attentive to their emotions, using the avatar 120.

The consideration information may be information related to consumers, and the control device 400 provides at least one of information on consumer rights, laws, and regulations to a user. As a result, the control device 400 can provide information and advice on consumer rights, laws, and regulations, as well as support for consumer troubles and complaint handling, to users with consumer issues, while being attentive to their emotions, using the avatar 120.

The consideration information may be information related to debt, and the control device 400 conducts mental support conversation with a user and provides at least one of information on laws related to debt, information on administrative procedures related to debt, and information on professionals according to debt-related information to the user. As a result, the control device 400 can propose coping methods and provide information on laws and procedures related to debt and introductions to professionals for debt settlement to users with debt issues, while being attentive to their emotions, using the avatar 120.

The consideration information may be information related to custody, and the control device 400 conducts support conversation for a user's position and provides at least one of information on laws related to custody and information on administrative procedures related to custody to a user. As a result, the control device 400 can provide advice that takes into account the user's position and emotions, as well as information on laws and procedures related to custody, to users with custody issues, while being attentive to their emotions, using the avatar 120.

### Example 17 of Embodiment

Next, Example 17 of the embodiment will be described. Here, description focuses on differences from the above embodiments, and descriptions of the same configuration and processing as in the above embodiments are omitted. An example of the control system 1 according to this embodiment is schematically illustrated in FIG. 39.

Appearance of an avatar may be human-like, as in the avatars 120a to 120c, or may be a stuffed animal character, as in the avatar 120d. For example, the avatars 120a to 120c may act as psychosomatic physicians or trainers, nodding and listening like real psychosomatic physicians or trainers, thereby providing information on emotion management, training, and the like to users such as athletes or those on a diet.

In this disclosure, examples are described in which appropriate information and advice are provided as various actions to users who are training. Note that training includes mental training, physical training, and dietary therapy.

Specifically, the control device 400 is installed in training facilities, a user's home, and the like, and the avatar 120 provides various information by conversation, gestures, image display, and the like according to a training situation. The control device 400 may also cooperate with the linked devices 500 such as thermometers, blood pressure monitors, smartwatches, body composition monitors, and training equipment.

For example, the control device 400 recognizes a behavior of a user who is an athlete and determines the user's emotions. The control device 400 provides information such as displaying images of situations where a user is under pressure, such as an opening ceremony or entry of a tournament, or tournament bracket of an upcoming competition, on a display. The avatar 120 may appear as a person modeled after a psychosomatic physician and, based on a behavior recognized and emotions determined by the control device 400, make statements such as “Let's take a deep breath” to provide coping methods for pressure. The avatar 120 may also appear as a person modeled after a trainer and, based on a behavior recognized and emotions determined by the control device 400, make statements such as “It's okay, you'll definitely succeed” to provide methods for increasing self-confidence. At this time, the avatar 120 reproduces facial expressions, hand movements, and the like as in actual medical examinations or training. That is, the control device 400 provides at least one of information on emotion management and information on mental training as training-related information to the user via the avatar 120 and the like.

The control device 400 also recognizes a behavior of a user who is on a diet and determines the user's emotions. For example, the control device 400 recognizes user's diet goals through conversation with the avatar 120 and recognizes the user's body shape, weight, and body fat percentage. The avatar 120 may appear as a person modeled after a trainer and, based on a behavior recognized and emotions determined by the control device 400, make statements such as “I will provide a reasonable diet plan” and display meal plans, exercise programs, and the like on a display. The Avatar 120 may also appear as a person modeled after a trainer and, based on a behavior recognized and emotions determined by control device 400, make statements such as “You're doing well” or “Let's keep going a little more” at predetermined timings such as 25%, 50%, or 75% progress in the diet plan, thereby supporting motivation maintenance and overcoming setbacks. That is, the control device 400 provides effective approaches for successful dieting via the avatar 120 and the like. In other words, the control device 400 provides at least one of information on meals and information on exercise as training-related information via the avatar 120 and the like.

The control device 400 also recognizes a behavior of a user who is training at home (for example, self-training) and determines the user's emotions. For example, the control device 400 recognizes a user's body shape and muscle strength (for example, obtained in cooperation with training equipment). The avatar 120 may appear as a person modeled after a trainer and, based on a behavior recognized and emotions determined by the control device 400, make statements such as “Try to imitate the correct form” and demonstrate a correct form with expressive gestures or display images of the correct form on a display. The avatar 120 may also appear as a person modeled after a trainer and, when the control device 400 recognizes that a user is training with a correct form, make statements such as “You're doing great” to praise the user. The avatar 120 may also appear as a person modeled after a trainer and, when a predetermined period such as one week or one month has passed, make statements such as “Your muscles are developing” to praise changes in the user's body shape. That is, the control device 400 provides information on training methods according to at least one of a user's body shape and muscle strength as training-related information via the avatar 120 and the like.

In this disclosure, the control device 400 can provide appropriate information and advice to users who are training via the avatar 120 and the like. That is, according to the control device 400 of this disclosure, it is possible to provide information according to a training situation.

A functional configuration of the control device 400 for controlling the avatar 120 is schematically illustrated in FIG. 40.

The speech understanding unit 222 analyzes user's voice detected by the microphone 211 and outputs character information representing user's speech content. For example, the speech understanding unit 222 outputs character information representing conversation content between avatar 120 and a user.

The state recognition unit 240 recognizes a state of a user based on information analyzed by the sensor module unit 220. For example, the state recognition unit 240 mainly performs processing related to perception using analysis results of the sensor module unit 220. For example, the state recognition unit 240 generates perceptual information such as “There is a 50% probability that the user is overcoming pressure” or “There is a 60% probability that the user is dissatisfied with the meal plan,” and performs processing to understand meaning of the generated perceptual information. For example, the state recognition unit 240 generates semantic information such as “The user has not achieved the training goal and motivation is declining.”

The behavior recognition unit 244 recognizes a behavior of a user based on information analyzed by the sensor module unit 220 and a state of the user recognized by the state recognition unit 240. For example, the information analyzed by the sensor module unit 220 and the recognized state of the user are input to a pre-trained neural network to obtain probabilities of each of a plurality of predefined behavior categories (for example, “laughing,” “angry,” “asking a question,” “sad”), and a behavior category with the highest probability is recognized as a behavior of the user. For example, the behavior recognition unit 244 recognizes behaviors such as “taking a deep breath,” “measuring weight and body fat percentage,” or “training” in training facilities or at home.

For example, the response rule 231 defines a behavior of the avatar 120 corresponding to behavior patterns such as when the user is taking a deep breath, measuring weight and body fat percentage, or training.

The behavior control unit 250 controls the avatar 120 and a controlled object based on a behavior of avatar 120 determined by the behavior determination unit 246. Specifically, the behavior control unit 250 controls a movement of avatar 120 and operation of a controlled object related to a movement of avatar 120 based on the behavior of avatar 120 associated with the response rule 231 determined by the behavior determination unit 246. For example, in a case where the avatar 120 and a user are having a conversation, if the behavior determination unit 246 determines a behavior including speech, the behavior control unit 250 controls the avatar 120 to take a predetermined action and causes a speaker included in the controlled object to output a voice of the avatar 120. At this time, the behavior control unit 250 may determine a speech speed based on an emotion value of the avatar 120. For example, the behavior control unit 250 determines a faster speech speed as an emotion value of the avatar 120 increases. In this way, the behavior control unit 250 determines an execution mode of a behavior determined by the behavior determination unit 246 based on the emotion value determined by the emotion determination unit 242. For example, the behavior control unit 250 controls avatar 120 to say “Did you calm down?” or “It's okay, you'll definitely succeed” when a user is taking a deep breath, “I will provide a reasonable diet plan” when a user is measuring weight and body fat percentage, and display meal plans or exercise programs on a display. The behavior control unit 250 also controls the avatar 120 to say “You're doing well” or “Let's keep going a little more” at predetermined timings during a diet plan, and to praise the user when training is performed correctly or when body changes are recognized. When user's emotion is negative, such as “sorrow,” “discomfort,” “anxiety,” “sadness,” “worry,” or “emptiness,” the behavior control unit 250 controls the avatar 120 to say “Just a little more!” or “Don't give up!” to encourage and motivate the user.

An example of an operation flow related to determining a behavior of the avatar 120 in the control device 400 is schematically illustrated in FIG. 41. Here, description focuses on differences from Example 16, and descriptions of the same processing as in Example 16 are omitted.

First, in step S1600, the state recognition unit 240 recognizes a state of a user based on information analyzed by the sensor module unit 220. For example, the state recognition unit 240 generates perceptual information such as “There is a 50% probability that the user is overcoming pressure” or “There is a 60% probability that the user is dissatisfied with the meal plan,” and performs processing to understand meaning of the generated perceptual information. For example, the state recognition unit 240 generates semantic information such as “The user has not achieved the training goal and motivation is declining.”

In step S1603, the behavior recognition unit 244 recognizes a behavior category of a user based on information analyzed by the sensor module unit 220 and a state of a user recognized by the state recognition unit 240. For example, the behavior recognition unit 244 recognizes behaviors such as “taking a deep breath,” “measuring weight and body fat percentage,” or “training” in training facilities or at home.

In step S1605, the behavior control unit 250 controls the avatar 120 and a controlled object based on a behavior of the avatar 120 determined by the behavior determination unit 246. For example, the behavior control unit 250 controls the avatar 120 to perform actions such as actions related to taking a deep breath or measuring weight and body fat percentage, demonstration of correct form by gestures, display of correct form images on a display, and actions to praise a user.

As described above, the control device 400 includes a control unit that recognizes a behavior of a user who is training and determines the user's emotions, and, based on the recognized behavior and determined emotions, controls the avatar 120 to provide training-related information to the user. As a result, the control device 400 can provide information according to a training situation using the avatar 120.

The control device 400 also provides at least one of information on emotion management and information on mental training to users who are athletes. As a result, the control device 400 can support improvement of athletic performance using the avatar 120.

The control device 400 also provides at least one of information on meals and information on exercise to users who are training for dieting. As a result, the control device 400 can provide effective approaches for successful dieting using avatar 120.

The control device 400 also provides information on training methods according to at least one of a user's body shape and muscle strength to users who are training. As a result, the control device 400 can use the avatar 120 to demonstrate correct form by gestures and praise a user.

### Example 18 of Embodiment

Next, Example 18 of the embodiment will be described. Here, description focuses on differences from the above embodiments, and descriptions of the same configuration and processing as in the above embodiments are omitted. An example of the control system 1 according to this embodiment is schematically illustrated in FIG. 39.

Appearance of an avatar may be human-like, as in the avatars 120a to 120c, or may be a stuffed animal character, as in the avatar 120d. For example, the avatars 120a to 120c may act as a child or grandchild of an elderly user, nodding and listening like a real child or grandchild, thereby providing mental care through conversation, daily life support, and health management advice to the elderly user.

The linked device 500 is communicably connected to control device 400 via the communication network 20 and exchanges information with the control device 400. With this configuration, the linked device 500 can perform its own control, conversations with users, mental care through conversation, daily life support, and health management advice according to instructions from the control device 400.

In this disclosure, examples are described in which mental care through conversation, daily life support, and health management advice are provided as various actions to elderly users.

Specifically, the control device 400 is installed in nursing homes, hospitals, and the like, and the avatar 120 performs actions such as providing mental care, daily life support, and health management advice according to a user's mental state (psychological state) through conversation and vital signs. The control device 400 may also cooperate with the linked devices 500 such as thermometers, blood pressure monitors, smartwatches, body composition monitors, and training equipment.

For example, the avatar 120 may appear as a person modeled after a child or grandchild of a user (elderly person) and, according to a user's mental state, make statements such as “Let's move our bodies” or “Let's watch a relaxing video” to provide mental care. At this time, the control device 400 provides care according to conversation content and emotions (user/self) of avatar 120. For example, if user's emotion is positive, such as “joy,” “pleasure,” or “excitement,” the avatar 120 makes statements such as “Let's move our bodies” and encourages exercise in cooperation with training equipment. On the other hand, if user's emotion is negative, such as “sorrow,” “discomfort,” “anxiety,” “sadness,” “worry,” or “emptiness,” the control device 400 has the avatar 120 appear as a person modeled after the user's child or grandchild and, for example, make statements such as “Let's watch a relaxing video” and encourage relaxation by cooperating with terminal devices, televisions, displays, and so on. That is, the control device 400 performs actions to support the user's daily life via the avatar 120 and the like according to the user's situation.

The control device 400 may also have the avatar 120 appear as a person modeled after a user's child or grandchild and, for example, make statements such as “Let's measure your blood pressure this morning” at a predetermined time every morning to encourage a user to measure body temperature, blood pressure, weight, and so on. The control device 400 may also have the avatar 120 appear as a person modeled after a user's child or grandchild and, for example, make statements such as “Let's start exercising” or “Let's go for a walk” at specific times in the morning and afternoon to encourage the user to exercise. The control device 400 may also have the avatar 120 appear as a person modeled after a user's child or grandchild and, for example, make statements such as “It's morning, let's get up” at a predetermined time every morning to support the user's daily rhythm. The control device 400 may also have the avatar 120 appear as a person modeled after a user's child or grandchild and, for example, make statements such as “Let's use the restroom before going to bed” before bedtime to support the user's daily rhythm. That is, the control device 400 performs actions to support the user's daily life via the avatar 120 and the like.

The control device 400 may also have the avatar 120 appear as a person modeled after a user's child or grandchild and, for example, make statements such as “Let's increase your activity” or “Let's avoid staying up late” according to trends in a user's body temperature, blood pressure, weight, and so on over a specific period such as a week or a month, thereby providing health management advice.

In this disclosure, the control device 400 can perform actions such as mental care through conversation, daily life support, and health management advice for a user via the avatar 120 and the like. That is, according to the control device 400 of this disclosure, it is possible to provide care according to a user's mental state.

A functional configuration of the control device 400 for controlling the avatar 120 is schematically illustrated in FIG. 40.

The behavior recognition unit 244 recognizes a behavior of a user based on information analyzed by the sensor module unit 220 and a state of the user recognized by the state recognition unit 240. For example, the information analyzed by the sensor module unit 220 and a recognized state of a user are input to a pre-trained neural network to obtain the probabilities of each of a plurality of predefined behavior categories (for example, “laughing,” “angry,” “asking a question,” “sad”), and a behavior category with the highest probability is recognized as a behavior of the user. For example, the behavior recognition unit 244 recognizes behaviors such as “not participating in group activities,” “restlessness,” or “looking down” in nursing homes or hospitals.

For example, the response rule 231 defines a behavior of the avatar 120 corresponding to behavior patterns such as when a user is not participating in group activities, is restless, or is looking down.

The behavior control unit 250 controls the avatar 120 and a controlled object based on a behavior of the avatar 120 determined by the behavior determination unit 246. Specifically, the behavior control unit 250 controls a movement of the avatar 120 and an operation of the controlled object related to a movement of the avatar 120 based on a behavior of the avatar 120 associated with the response rule 231 determined by the behavior determination unit 246. For example, in a case where the avatar 120 and a user are having a conversation, if the behavior determination unit 246 determines a behavior including speech, the behavior control unit 250 controls the avatar 120 to take a predetermined action and causes a speaker included in the controlled object to output voice of the avatar 120. At this time, the behavior control unit 250 may determine a speech speed based on an emotion value of the avatar 120. For example, the behavior control unit 250 determines a faster speech speed as an emotion value of the avatar 120 increases. In this way, the behavior control unit 250 determines an execution mode of the behavior determined by the behavior determination unit 246 based on the emotion value determined by the emotion determination unit 242. For example, the behavior control unit 250 controls avatar 120 to say “Would you like to join the recreation?” or “Are you tired? Would you like to watch a relaxing video over there?” when a user is not participating in group activities, thereby guiding the user to care for their mental state. The behavior control unit 250 also controls the avatar 120 to say “Is there something on your mind?” when the user is restless, and to say “Are you okay?” when the user is looking down, and to notify a care manager or nurse if there are abnormal vital signs. When user's emotion is negative, such as “sorrow,” “discomfort,” “anxiety,” “sadness,” “worry,” or “emptiness,” the behavior control unit 250 controls the avatar 120 to say “The nurse will be here soon, so please don't worry” to ease the user's negative emotions.

An example of an operation flow related to determining a behavior of the avatar 120 in the control device 400 is schematically illustrated in FIG. 41. Here, description focuses on differences from Example 16, and descriptions of the same processing as in Example 16 are omitted.

First, in step S1600, the state recognition unit 240 recognizes a state of a user based on information analyzed by the sensor module unit 220. For example, the state recognition unit 240 generates perceptual information such as “There is a 70% probability that the user is mentally fatigued” or “There is a 30% probability that the user is homesick” as a user's mental state, and performs processing to understand meaning of generated perceptual information. For example, the state recognition unit 240 generates semantic information such as “The user's mental state is weak, and care that provides a sense of security is needed.”

In step S1603, the behavior recognition unit 244 recognizes a behavior category of a user based on information analyzed by the sensor module unit 220 and a state of the user recognized by the state recognition unit 240. For example, the behavior recognition unit 244 recognizes behaviors such as “not participating in group activities,” “restlessness,” or “looking down” in nursing homes or hospitals.

In step S1605, the behavior control unit 250 controls the avatar 120 and a controlled object based on a behavior of the avatar 120 determined by the behavior determination unit 246. For example, the behavior control unit 250 controls the avatar 120 to perform actions such as guiding a user to participate in group activities, supporting a user including mental care when the user is restless or looking down, and so on.

As described above, the control device 400 includes a control unit that recognizes a user's mental state and determines the user's emotions, and, based on the determined emotions, conducts a conversation with the user and controls the avatar 120 to provide care according to the recognized mental state. As a result, the control device 400 can provide care according to a user's mental state using the avatar 120.

The control device 400 may further control the avatar 120 to support a user's daily life. As a result, the control device 400 can use the avatar 120 to create an environment where a user can live with peace of mind.

The control device 400 may further control the avatar 120 to provide health management advice to a user. As a result, the control device 400 can use the avatar 120 to create an environment where a user can live healthily.

A user is an elderly person. As a result, the control device 400 can use the avatar 120 to provide care according to a mental state of the elderly person and create an environment where the elderly person can live with peace of mind and health.

### Example 19 of Embodiment

Next, Example 19 of the embodiment will be described. Here, description focuses on differences from the above embodiments, and descriptions of the same configuration and processing as in the above embodiments are omitted.

FIG. 42 is a diagram schematically illustrating an example of the control system 1 according to the nineteenth embodiment. As shown in FIG. 42, the control system 1 includes a control device 600, which is an avatar display device, a plurality of avatars 700, the linked device 500, and the server 300. The plurality of avatars 700 are managed by respective users. The control device 600 is a device capable of displaying the avatars 700. For example, FIG. 42 shows an example in which the control device 600 displays the avatar 700 on a display monitor, but this is not limiting. In this embodiment, a device for displaying the avatar 700 may include a monitor, a display unit of a notebook PC or mobile terminal, a wearable terminal device related to augmented reality, virtual reality, or mixed reality, such as VR/AR goggles, VR/AR glasses, or other wearable devices that provide information perceptible by the user's five senses.

Appearance of an avatar may be human-like, as in the avatar 700, or may be a character. For example, the avatar 700 may act as a counselor, nodding and listening like a real counselor, thereby providing emotional support and appropriate coping methods or self-care methods for workplace concerns and stress to business persons with labor issues. For example, the avatar 700, by having a character-like appearance, is considered to be particularly familiar to children.

Descriptions of the avatar 700 in this embodiment that are the same as those for the avatar 100 in the above embodiments are omitted. Descriptions of the control device 600 in this embodiment that are the same as those for the control device 200 in the above embodiments are also omitted.

The avatar 700 can also perform actions in cooperation with a linked device 800. The linked device 800 may be, for example, karaoke equipment, wine cellar, refrigerator, terminal device (PC, smartphone, tablet, etc.), washing machine, automobile, camera, toilet equipment, electric toothbrush, television, display, furniture (closet, etc.), medicine box, musical instrument, lighting equipment, exercise toys (such as a unicycle), and so on. These linked devices 800 are communicably connected to the control device 600 via the communication network 20 and exchange information with the control device 600. With this configuration, the linked device 800 can perform its own control and conversations with users according to instructions from the avatar 700.

In this disclosure, an example is described in which the control device 600 analyzes emotions of a user and emotions of other users, determines a behavior of the avatar 700 corresponding to emotions of the user and the other users, and controls a controlled object based on the determined behavior of the avatar 700.

For example, the avatar 700 analyzes that user's emotion is “anger” and other user's emotion is “confusion,” and, in order to calm the user's “anger,” provides advice to the other user on information or responses (such as how to resolve misunderstandings or how to apologize) estimated to be a cause of the user's “anger,” corresponding to the other user's “confusion.”

The control device 600 may also determine actions to improve interpersonal skills of other users based on emotions of a user and other users. For example, when user's emotion is “joy” and other user's emotion is “boredom,” the avatar 700 provides advice to the other user on behaviors such as showing empathy for the user's “joy,” and teaches appropriate responses. For example, the avatar 700 advises the other user on gestures or body language to show empathy for the user's “joy.”

The control device 600 may also determine actions to teach other users behaviors that increase a user's emotion value. For example, when user's emotion is “sorrow,” the avatar 700 advises another user to listen quietly or encourage the user, thereby teaching behaviors that increase a user's emotion value.

The control device 600 may also determine actions to improve relationship between a user and another user based on emotions of the user and the other user. For example, when both the user and the other user have emotion “anger,” the avatar 700 analyzes causes of anger and what each cannot forgive, and provides advice for problem solving, such as sharing analysis results and suggesting communication tips.

The control device 600 may also determine actions to make a user or another user understand at least one of user's emotion or other user's emotion. For example, when the user's emotion is “anger” and the other user's emotion is “joy,” and the other user is unaware of the user's “anger,” the avatar 700 gently conveys the user's emotion to the other user. Similarly, when the user's emotion is “sadness” and the other user's emotion is “anger,” the avatar 700 conveys to the other user, in a way that can be accepted in an “angry” state, that the user is feeling sad or what the other user is angry about.

The control device 600 may also analyze emotions of a user and emotions of other users who live at a predetermined distance from the user's residence. For example, the avatar 700 analyzes the emotions of the user and the emotions of another user who lives far from the user's residence and is in a romantic relationship with the user, and provides advice for maintaining and strengthening the romantic relationship. For example, the avatar 700 predicts possible problems (such as quarrels) from the user's emotion “loneliness” and the other user's emotion “happiness” in a long-distance relationship, and acts to mediate between the two by encouraging understanding and sharing of emotions to prevent problems from occurring.

The control device 600 may also determine actions to provide predetermined information to other users based on user's emotion, information about a user, and emotions of other users. For example, the avatar 700, based on user's emotion “expectation,” user's interests and hobbies, and other user's emotion “anxiety,” conveys to the other user what kind of present the user is expecting in a way that encourages the other user in an “anxious” state.

The control device 600 may also determine actions to provide predetermined information to other users based on at least one of user's past information, interests, concerns, hobbies, preferences, orientations, or specific anniversaries as information about the user. For example, the avatar 700, based on user's emotion “boredom,” user's past information “the trigger for deepening relationship with other user,” and the other user's emotion “indecision,” proposes a present that makes the user feel loved and encourages the other user to make a decision while in an “indecisive” state.

In this way, in this disclosure, the avatar 700 can improve relationships between users.

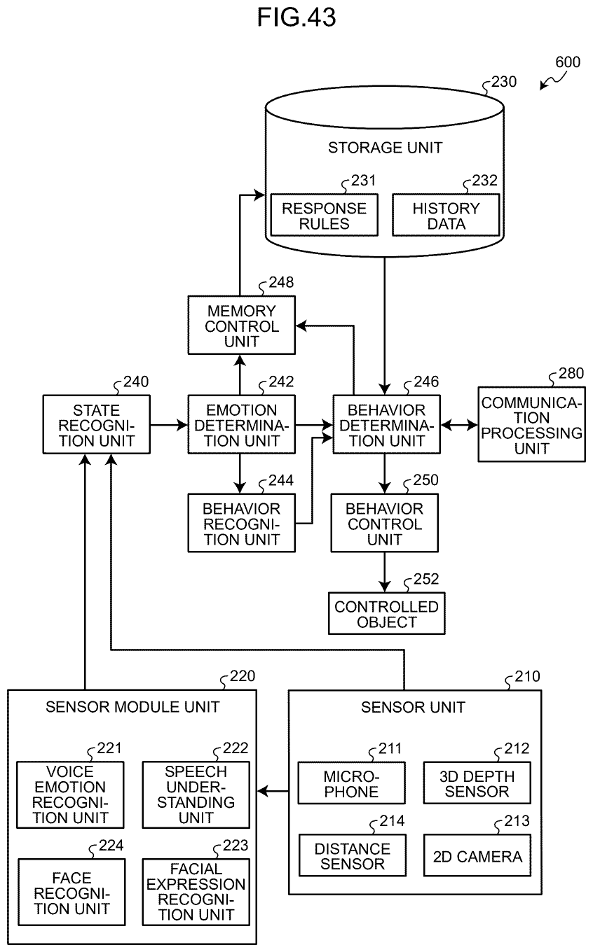

FIG. 43 is a diagram schematically illustrating a functional configuration of the control device 600. The control device 600 includes a control unit having the sensor unit 210, the sensor module unit 220, the storage unit 230, the state recognition unit 240, the emotion determination unit 242, the behavior recognition unit 244, the behavior determination unit 246, the memory control unit 248, the behavior control unit 250, the controlled object 252, and the communication processing unit 280.

The sensor unit 210 includes the microphone 211, the 3D depth sensor 212, the 2D camera 213, and the distance sensor 214. The sensor unit 210 may also include an acceleration sensor and a thermo sensor. The sensor unit 210 may also include a clock, a touch sensor, and others.

At least a part of the storage unit 230 is implemented by a storage medium such as memory. A person DB, which stores face images and attribute information of users, may also be included. The person DB may also include information about a user, such as the user's past information, interests, concerns, hobbies, preferences, orientations, and specific anniversaries.

The facial expression recognition unit 223 recognizes facial expression and emotion of a user from images of the user captured by the 2D camera 213. For example, the facial expression recognition unit 223 recognizes facial expression and emotion of a user who receives words and actions of other users. The facial expression recognition unit 223 may also recognize facial expression and emotion of other users who receive words and actions of a user.

The state recognition unit 240 recognizes a state of a user based on information analyzed by the sensor module unit 220. For example, the state recognition unit 240 mainly performs processing related to perception using analysis results of the sensor module unit 220. For example, the state recognition unit 240 generates perceptual information such as “The user is talking with another user,” “The user is laughing,” or “There is an XX % probability that the user is enjoying the conversation,” and performs processing to understand meaning of the generated perceptual information. For example, the state recognition unit 240 generates semantic information such as “The user is laughing at the other user's story and seems to be enjoying the conversation.”

The emotion determination unit 242 may also determine an emotion value of the avatar 700 by further considering a state of the avatar 700. For example, when a surrounding environment of the avatar 700 is completely dark, the emotion determination unit 242 may increase a “sorrow” emotion value of the avatar 700. If a user continues to talk to the avatar 700 even though a surrounding environment is completely dark, an “anger” emotion value may also be increased.

The behavior recognition unit 244 recognizes behavior of a user based on information analyzed by the sensor module unit 220 and a state of a user recognized by the state recognition unit 240. For example, information analyzed by the sensor module unit 220 and a recognized state of a user are input to a pre-trained neural network to obtain probabilities of each of a plurality of predefined behavior categories (for example, “laughing,” “angry,” “asking a question,” “sad”), and a behavior category with the highest probability is recognized as the behavior of the user. For example, the behavior recognition unit 244 recognizes behaviors such as “talking,” “listening,” or “physical contact” between the user and other users.

For example, the response rule 231 defines a behavior of the avatar 700 corresponding to behavior patterns such as when a user is talking, listening, or making requests (such as “liven things up” or “calm down”).

The behavior control unit 250 controls the controlled object 252 based on a behavior determined by the behavior determination unit 246. For example, when the behavior determination unit 246 determines a behavior including speech, the behavior control unit 250 causes a speaker included in the controlled object 252 to output sound. At this time, the behavior control unit 250 may determine a speech speed based on an emotion value of the avatar 700. For example, the behavior control unit 250 determines a faster speech speed as an emotion value of the avatar 700 increases. In this way, the behavior control unit 250 determines an execution mode of a behavior determined by the behavior determination unit 246 based on an emotion value determined by the emotion determination unit 242.

For example, the behavior control unit 250 determines its own behavior corresponding to emotions of a user and other users, and controls a controlled object based on the determined behavior. For example, when analyzed emotion of a user is “anger” and emotion of another user is “confusion,” the behavior control unit 250 provides advice to the other user on information or responses (such as how to resolve misunderstandings or how to apologize) estimated to be a cause of the user's “anger,” corresponding to the other user's “confusion.”

The behavior control unit 250 may also determine actions to improve interpersonal skills of other users based on emotions of a user and other users. For example, when a user's emotion is “joy” and other user's emotion is “boredom,” the behavior control unit 250 provides advice to the other user on behaviors such as showing empathy for the user's “joy,” and teaches appropriate responses. For example, the behavior control unit 250 advises the other user on gestures or body language to show empathy for the user's “joy.”

The behavior control unit 250 may also determine actions to teach other users behaviors that increase a user's emotion value. For example, when user's emotion is “sorrow,” the behavior control unit 250 advises the other user to listen quietly or encourage the user, thereby teaching behaviors that increase a user's emotion value.

The behavior control unit 250 may also determine actions to improve relationship between user and other users based on emotions of the user and other users. For example, when both the user and the other user have emotion “anger,” the behavior control unit 250 analyzes causes of anger and what each cannot forgive, and provides advice for problem solving, such as sharing analysis results and suggesting communication tips.

The behavior control unit 250 may also determine actions to make a user or another user understand at least one of the user's emotion or the other user's emotion. For example, when a user's emotion is “anger” and another user's emotion is “joy,” and the other user is unaware of the user's “anger,” the behavior control unit 250 gently conveys the user's emotion to the other user. Similarly, when the user's emotion is “sadness” and the other user's emotion is “anger,” the behavior control unit 250 conveys to the other user, in a way that can be accepted in an “angry” state, that the user is feeling sad or what the other user is angry about.

The behavior control unit 250 may also analyze emotions of a user and emotions of other users who live at a predetermined distance from the user's residence. For example, the behavior control unit 250 analyzes emotions of a user and emotions of another user who lives far from the user's residence and is in a romantic relationship with the user, and provides advice for maintaining and strengthening romantic relationship. For example, the behavior control unit 250 predicts possible problems (such as quarrels) from the user's emotion “loneliness” and the other user's emotion “happiness” in a long-distance relationship, and acts to mediate between the two by encouraging understanding and sharing of emotions to prevent problems from occurring.

The behavior control unit 250 may also determine actions to provide predetermined information to other users based on the user's emotion, information about the user, and emotions of other users. For example, the behavior control unit 250, based on user's emotion “expectation,” the user's interests and hobbies, and the other user's emotion “anxiety,” conveys to the other user what kind of present the user is expecting in a way that encourages the other user in an “anxious” state.

The behavior control unit 250 may also determine actions to provide predetermined information to other users based on at least one of user's past information, interests, concerns, hobbies, preferences, orientations, or specific anniversaries as information about the user. For example, the behavior control unit 250, based on user's emotion “boredom,” the user's past information “the trigger for deepening the relationship with the other user,” and the other user's emotion “indecision,” proposes a present that makes the user feel loved and encourages the other user to make a decision while in an “indecisive” state.

An example of an operation flow related to determining a behavior of the avatar 700 is schematically illustrated in FIG. 41. Here, description focuses on differences from Example 16, and descriptions of the same processing as in Example 16 are omitted.

First, in step S1600, the state recognition unit 240 recognizes a state of a user based on information analyzed by the sensor module unit 220. The state recognition unit 240 generates perceptual information such as “The user is talking with another user,” “The user is laughing,” or “There is an XX % probability that the user is enjoying the conversation,” and performs processing to understand meaning of the generated perceptual information. For example, the state recognition unit 240 generates semantic information such as “The user is laughing at the other user's story and seems to be enjoying the conversation.”

In step S1603, the behavior recognition unit 244 recognizes a behavior category of a user based on information analyzed by the sensor module unit 220 and a state of the user recognized by the state recognition unit 240. For example, the behavior recognition unit 244 recognizes behaviors such as “talking,” “listening,” or “physical contact.”

In step S1605, the behavior control unit 250 controls the controlled object 252 based on behavior determined by the behavior determination unit 246. For example, the behavior control unit 250 performs actions to improve relationship between a user and other users.

As described above, the avatar 700 includes a control unit that analyzes emotions of a user and other users, determines its own behavior corresponding to emotions of the user and other users, and controls a controlled object based on the determined behavior. As a result, the avatar 700 can determine actions corresponding to emotions of the user and other users and improve relationships between the users.

The control unit of the control device 600 may also determine actions to improve interpersonal skills of other users based on emotions of a user and other users. As a result, the avatar 700 can advise other users on behaviors corresponding to emotions of a user and other users, teach appropriate responses, and improve interpersonal skills of the other users.

The control unit of the control device 600 may also determine actions to teach other users behaviors that increase a user's emotion value. As a result, the avatar 700 can advise other users on behaviors to increase an analyzed user's emotion value, teach appropriate responses, and improve relationships between users.

The control unit of the control device 600 may also determine actions to improve relationship between a user and other users based on emotions of the user and the other users. As a result, the avatar 700 can use emotions of the user and the other users to provide advice to other users who want to become closer to the user, according to each emotion, and improve relationships between users.

The control unit of the control device 600 may also determine actions to make a user or another user understand at least one of the user's emotion or the other user's emotion. As a result, the avatar 700 can make the user or the other user understand the other's or their own emotions, and improve relationships between users.

The control unit of the control device 600 may also analyze emotions of a user and emotions of other users who live at a predetermined distance from the user's residence. As a result, the avatar 700 can, for example, analyze emotions of users in a long-distance romantic relationship, act to prevent problems from occurring based on the analyzed emotions, maintain relationship, and act to further enhance the relationship.

The control unit of the control device 600 may also determine actions to provide predetermined information to other users based on user's emotion, information about a user, and emotions of the other users. As a result, the avatar 700 can propose the optimal present for a favorite person or partner by using the other's emotions and attribute information, and provide support tailored to the user by using emotions of both parties, thereby improving relationships between users.

A control unit of the control device 600 may also determine actions to provide predetermined information to other users based on at least one of user's past information, interests, concerns, hobbies, preferences, orientations, or specific anniversaries as information about the user. As a result, the avatar 700 can propose an optimal present for a favorite person or partner by using other's emotions, past information, hobbies, preferences, anniversaries, and so on, and provide support tailored to a user by using emotions of both parties, thereby improving relationships between users.

### Example 20 of Embodiment

Next, Example 20 of the embodiment will be described. Here, description focuses on differences from the above embodiments, and descriptions of the same configuration and processing as in the above embodiments are omitted.

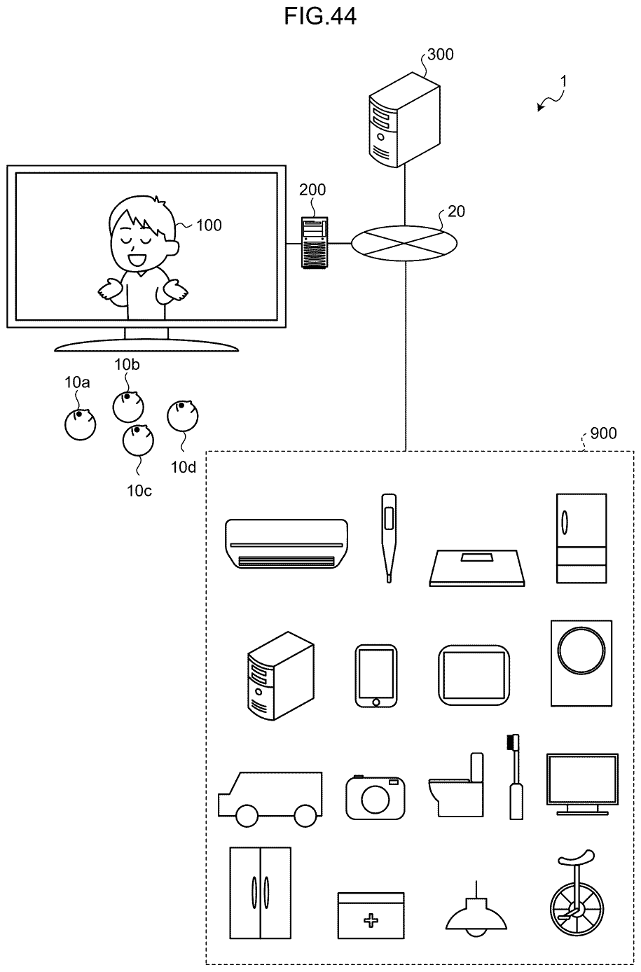

FIG. 44 is a diagram schematically illustrating an example of the control system 1 according to the twentieth embodiment. As shown in FIG. 1, the control system 1 includes the control device 200 for controlling avatars such as the avatar 100, a linked device 900, and the server 300.

Appearance of the avatar 100 may be human-like or may be modeled after a stuffed animal. For example, the avatar 100 may act as a counselor or doctor, nodding and listening like a real counselor or doctor, thereby making it easier for the user 10 to consult the avatar 100 about their concerns. The avatar 100 may also be a child avatar modeled after a child, making it easier for the user 10 to feel familiar with the avatar 100.

The control device 200 can control the avatar 100 in cooperation with the linked device 900. The linked device 900 may be, for example, IoT home appliances, IoT devices, or other communicable devices. Examples of the linked device 900 include air conditioners, IoT health devices (thermometers, scales), refrigerators, terminal devices (PCs, smartphones, tablets, etc.), washing machines, automobiles, cameras, toilet equipment, electric toothbrushes, televisions, displays, furniture (closets, etc.), medicine boxes, lighting equipment, and exercise toys (such as a unicycle). The linked devices 900 are communicably connected to the control device 200 via the communication network 20 and exchange information with the control device 200. With this configuration, the linked device 900 can perform its own control and conversations with the user 10 according to instructions from the control device 200.

In this disclosure, an example is described in which the avatar 100 performs various actions for the user 10 by cooperating with the linked devices 900 such as air conditioners and IoT health devices (thermometers, scales) and the control device 200. The linked device 900 measures values related to a physical condition of the user 10 and sends the measured data to the server 300 or the control device 200. For example, the linked device 900 may be a thermometer or scale. The linked device 900 sends temperature or weight data.

The control device 200 provides childcare support for users such as parents of infants via the avatar 100. For example, the control device 200 proposes methods for responding to emotions and behaviors of children via the avatar 100. The control device 200 may also provide childcare support in cooperation with the linked device 900.

Specifically, the control device 200 collects data related to a physical condition of the user 10 from IoT health devices, which are the linked devices 900. For example, each the user 10, including children, is assigned identification information such as an ID in advance. The IoT health device measures values related to a physical condition of the user 10 by inputting identification information. For example, a thermometer or scale measures temperature or weight of a child by inputting the child's identification information. The control device 200 collects temperature or weight data corresponding to the child's identification information from thermometer or scale.

The control device 200 recognizes a state of a child from collected data and estimates the child's emotions based on the recognized state. The control device 200 provides support according to the estimated emotions of the child.

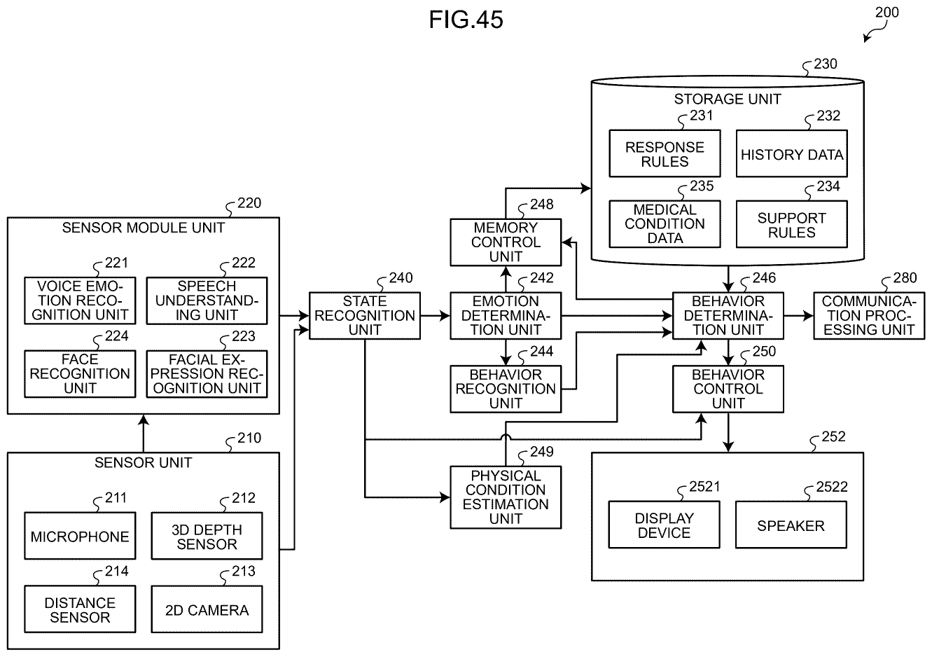

FIG. 45 is a diagram schematically illustrating a functional configuration of the control device 200 for controlling the avatar 100. The control device 200 includes the sensor unit 210, the sensor module unit 220, the storage unit 230, the state recognition unit 240, a physical condition estimation unit 249, the emotion determination unit 242, the behavior recognition unit 244, the behavior determination unit 246, the memory control unit 248, the behavior control unit 250, the controlled object 252, and the communication processing unit 280.

The storage unit 230 includes the response rules 231, the history data 232, medical condition data 235, and support rules 234. The medical condition data 235 stores information about medical conditions for each disease. The support rules 234 are rules that define actions to be supported by the avatar 100. The support rules 234 store information related to content to be responded to or supported by the avatar 100 for actions of the user 10 for each physical condition of a child. The storage unit 230 may also include the person DB that stores face images and attribute information of the user 10. The person DB may further include face images of family members such as parent, spouse, and child of the user 10.

The control device 200 also provides various childcare support for the user 10 via the avatar 100. For example, when data about a child is collected from the linked device 500, the state recognition unit 240 recognizes a state of the child based on the collected data. The emotion determination unit 242 determines an emotion value of the child based on the data collected from the linked device 500 and a state of the child recognized by the state recognition unit 240. For example, the emotion determination unit 242 inputs the collected data and the recognized state of the child into a pre-trained neural network to obtain an emotion value of the child.

The control device 200 may also recognize a state of a child and estimate the child's emotions based on information detected by the sensor unit 210. For example, the state recognition unit 240 determines whether the child is included in video captured by the 2D camera 213 provided in the sensor unit 210. If the child is included in the video, the state recognition unit 240 recognizes a state of the child based on information analyzed by the sensor module unit 220. The state recognition unit 240 may also detect a body temperature of the user 10 using a thermography sensor provided in the sensor unit 210.

The state recognition unit 240 may also recognize a state of the user 10 or a child by recognizing audio collected by the microphone 211. For example, if the user 10 inputs by voice, “My child has a fever,” “My child has a sore throat,” or “My child has a stomachache,” the state recognition unit 240 recognizes a state of a child from recognition result of the audio collected by the microphone 211.

The physical condition estimation unit 249 estimates a child's physical condition from a recognized state of a child based on the medical condition data 235. For example, the physical condition estimation unit 249 estimates a child's physical condition from a child's temperature or weight based on the medical condition data 235. If the medical condition data 235 includes a disease corresponding to a child's state, the physical condition estimation unit 249 estimates that a child is in a state of the corresponding disease. If the physical condition estimation unit 249 recognizes a state of a child from voice of the user 10, it may also use a state of a child recognized from voice to estimate the child's physical condition.

The behavior determination unit 246 determines a behavior based on at least one of child's emotions and a physical condition when at least one of child's emotions and a physical condition is required. For example, when child's emotions are required, the behavior determination unit 246 determines a behavior of the avatar 100 according to the child's emotions. When a child is estimated to be ill, the behavior determination unit 246 determines a behavior of the avatar 100 according to the child's illness. For example, the behavior determination unit 246 reads out information related to a response or support content corresponding to at least one of child's emotions and a physical condition from the support rules 234. The behavior determination unit 246 determines a behavior of the avatar 100 based on the read information.

The support rules 234 store information related to a content to be responded to or supported for each of child's emotions and physical conditions, and for each issue, for the user 10. For example, the support rules 234 store information on how to respond to a child for each issue according to the child's emotions. For example, the support rules 234 store advice on how to respond to child's emotions, how to manage parent's own emotions, and how to reduce stress for each issue according to child's emotions. The support rules 234 also store information on how to respond to the child's emotions and behaviors for each issue according to the child's physical condition. For example, the support rules 234 store information on necessary care, appropriate medical department to visit, and likely effective medications for each disease. The support rules 234 also store information on appropriate medical department to visit for each disease. The support rules 234 also store information on likely effective medications for each disease. The support rules 234 also store information on an appropriate indoor environment for a child. The support rules 234 also store information on music suitable for putting a child to sleep.

When the behavior determination unit 246 estimates that a child is ill, it provides support according to a child's illness. The behavior determination unit 246 according to this embodiment identifies issues from speech content of the user 10. The behavior determination unit 246 analyzes character information representing the speech content of the user 10 to identify issues included in the speech content. The behavior determination unit 246 reads out information related to a response or support content corresponding to child's emotions, physical condition, and identified issues from the support rules 234. The behavior determination unit 246 determines a behavior of the avatar 100 based on the read information.

The behavior determination unit 246 may also control the linked device 900 based on the support rules 234. For example, the behavior determination unit 246 reads out information on an appropriate indoor environment for a child from the support rules 234 and controls an air conditioner, which is the linked device 900, to create an appropriate indoor environment for the child. For example, the behavior determination unit 246 controls temperature setting of an air conditioner to create an appropriate indoor environment for the child. The behavior determination unit 246 also reads out information on music suitable for putting a child to sleep from the support rules 234 and determines a behavior of the avatar 100 to output music suitable for putting the child to sleep from the speaker 2522.

The behavior control unit 250 may control the controlled object 252 to express emotions according to an estimated emotion of the user 10 in synchronization with a recognized state of the user 10. For example, in the embodiment, the avatar 100 detects the user 10 by audio or images using the sensor unit 210, the sensor module unit 220 analyzes information, and the state recognition unit 240 recognizes a state of the user 10 based on the analysis. The emotion determination unit 242 estimates emotion of the user 10 by determining an emotional state of the user 10 from the recognized state. The behavior control unit 250 controls the controlled object 252 to express emotions according to the estimated emotion of the user 10 in synchronization with a state of the user 10 recognized by the state recognition unit 240. If the emotion of the user 10 estimated by the emotion determination unit 242 is positive, the behavior control unit 250 controls the controlled object 252 to express emotions according to the estimated emotion of the user 10 in synchronization with a state of the user 10 recognized by the state recognition unit 240. For example, if emotion values for bright emotions such as “joy,” “pleasure,” “comfort,” “relief,” “excitement,” “ease,” and “fulfillment” are large, and the emotion values for unpleasant emotions such as “anger,” “sorrow,” “discomfort,” “anxiety,” “sadness,” “worry,” and “emptiness” are small, the state is determined to be positive. Conversely, if emotion values for bright emotions are small and the emotion values for unpleasant emotions are large, a state is determined to be negative. If emotion of the user 10 is positive, the behavior control unit 250 controls the controlled object 252 to express emotions according to estimated emotion of the user 10 in the same state as the user 10. For example, the state recognition unit 240 determines a speech speed based on an emotion value of the user 10. The state recognition unit 240 may also recognize features such as gestures, speaking style, or word choice of the user 10 by detecting the user 10 by audio or image using the sensor unit 210. The behavior control unit 250 controls the controlled object 252 to express emotions according to estimated emotion of the user 10, using the same features as the user 10 for gestures, speaking style, or word choice. The state recognition unit 240 may also recognize facial expressions, voice tone, or nuances of words of the user 10. The behavior control unit 250 controls the controlled object 252 to express emotions according to the estimated emotion of the user 10, using the same state as the user 10 for facial expressions, voice tone, or nuances of words. By expressing emotions according to emotions of the user 10 in this way, the avatar 100 can enhance the psychological connection and relationship with the user 10.

The control device 200 also provides childcare support for users such as parents of infants via the avatar 100. For example, when data about a child is collected from the linked device 900 or information about a child is detected by the sensor unit 210, the control device 200 recognizes a state of the child based on data collected from the linked device 900 or information detected by the sensor unit 210, and estimates the child's emotions based on the recognized state. The control device 200 provides support according to the estimated emotions of the child. For example, the control device 200 proposes methods for responding to child's emotions, physical condition, and issues identified from speech content of the user 10 via the avatar 100, such as advice on how to respond to a child, how to manage parent's own emotions, and how to reduce stress. The control device 200 may also autonomously control IoT home appliances to maintain a comfortable indoor environment. For example, the control device 200 controls temperature setting of an air conditioner, which is the linked device 900, to create an appropriate indoor environment for a child. The control device 200 may also utilize function of the speaker 2522 included in the controlled object 252 to play music effective for putting infants to sleep. In this way, the control device 200 can support and assist users (parents) raising children. As a result, the control device 200 can provide support to maximize joy of parenting.

The control device 200 may also provide childcare support by sending various information to information terminals such as smartphones possessed by the user 10. The control device 200 may also monitor a state and a physical condition of a child as needed and provide childcare support by sending the state and physical condition of the child to the information terminals possessed by the parent.

The control device 200 may also display a child avatar when it recognizes a state of a child of the user 10 who communicates with the avatar 100, and control a child avatar to express at least one of the child's state and emotions. For example, the behavior control unit 250 may display a child avatar resembling a child of the user 10 and have the child avatar speak on behalf of the real child about what they want the parent to do or express the real child's current emotions. For example, the behavior control unit 250 may read out a child's face image from the person DB (not shown) and display a child avatar using the child's face image for the avatar's face, and have the child avatar speak about at least one of the child's state and emotions. The behavior control unit 250 may also have a child avatar speak support content according to child's emotions. This allows parents to sense real child's emotions, which are often difficult to read, through a child avatar.

The behavior control unit 250 may also determine a speech speed based on an emotion value of the avatar 100. For example, if emotion of the user 10 is positive, the behavior control unit 250 determines a speech speed based on an emotion value of the avatar 100. For example, the behavior control unit 250 determines a faster speech speed as an emotion value of the avatar 100 increases. In this way, the behavior control unit 250 determines an execution mode of a behavior determined by the behavior determination unit 246 based on an emotion value determined by the emotion determination unit 242.

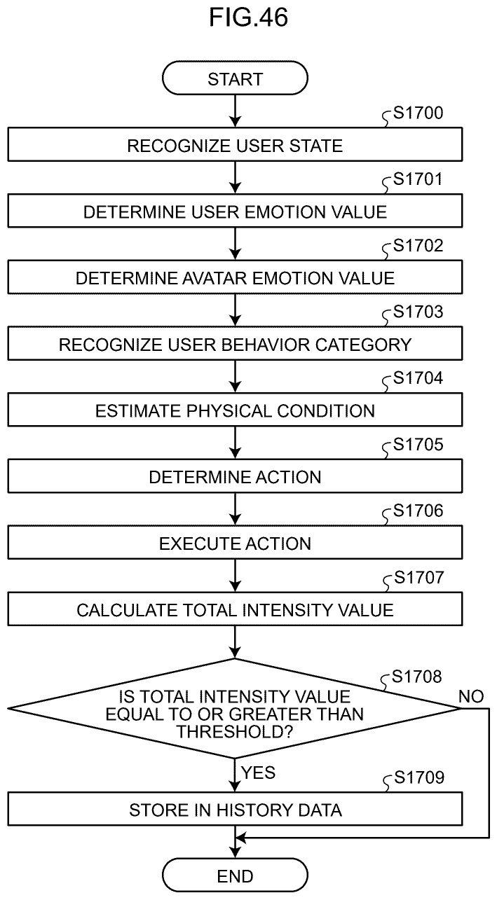

FIG. 46 is a diagram schematically illustrating an example of an operation flow related to determining a behavior of the avatar 100. The operation flow shown in FIG. 46 is repeatedly executed. At this time, it is assumed that information analyzed by the sensor module unit 220 is being input.

First, in step S1700, a state of the user 10 who communicates with the avatar 100 is recognized. For example, the state recognition unit 240 recognizes a state of the user 10 who communicates with the avatar 100 based on information analyzed by the sensor module unit 220. The state recognition unit 240 may also recognize a state of a child based on data collected from the linked device 800 or information detected by the sensor unit 210 when data about the child is collected from the linked device 800 or information about the child is detected by the sensor unit 210.

In step S1701, emotion of the user 10 is estimated based on a recognized state of the user 10. When a state of a child is recognized, emotion of the child is estimated based on the recognized state of the child. For example, the emotion determination unit 242 determines an emotion value of the user 10 based on information analyzed by the sensor module unit 220 and a state of the user 10 recognized by the state recognition unit 240. The emotion determination unit 242 may also determine an emotion value of a child based on data collected from the linked device 800 and a state of a child recognized by the state recognition unit 240.

In step S1702, the emotion determination unit 242 determines an emotion value of the avatar 100 based on information analyzed by the sensor module unit 220 and a state of the user 10 recognized by the state recognition unit 240. The emotion determination unit 242 adds the determined emotion value of the user 10 to the history data 232.

In step S1703, the behavior recognition unit 244 recognizes a behavior category of the user 10 based on information analyzed by the sensor module unit 220 and a state of the user 10 recognized by the state recognition unit 240.

In step S1704, the physical condition estimation unit 249 estimates a physical condition of a child based on a recognized state of the child. For example, the physical condition estimation unit 249 estimates a physical condition of a child based on information recognized from audio collected by the microphone 211 and a state of the child recognized by the state recognition unit 240.

In step S1705, the behavior determination unit 246 determines a behavior of the avatar 100. For example, the behavior determination unit 246 determines a behavior of the avatar 100 based on a combination of the current emotion value of the user 10 determined in step S1701, the past emotion value of the user 10, an emotion value of the avatar 100, a behavior of the user 10 recognized by the behavior recognition unit 244, and the response rule 231. When at least one of child's emotions and physical condition is required in step S1702, the behavior determination unit 246 determines a behavior based on at least one of child's emotions and physical condition. For example, the behavior determination unit 246 reads out information related to a response or support content corresponding to at least one of the child's emotions and physical condition from the support rules 234. The behavior determination unit 246 determines a behavior of the avatar 100 based on the read information.

In step S1706, the behavior control unit 250 controls the controlled object 252 based on a behavior determined by the behavior determination unit 246. At this time, if emotion of the user 10 estimated by the emotion determination unit 242 is positive, the behavior control unit 250 controls the controlled object 252 to express emotions according to estimated emotion of the user 10 in synchronization with a state of the user 10 recognized by the state recognition unit 240. For example, if emotion of the user 10 is positive, the behavior control unit 250 controls the controlled object 252 to express emotions according to estimated emotion of the user 10 in the same state as the user 10. The behavior control unit 250 also determines a speech speed based on an emotion value of the avatar 100 if the emotion of the user 10 is positive.

In step S1707, the memory control unit 248 calculates a total value of intensities based on a predetermined intensity of a behavior determined by the behavior determination unit 246 and an emotion value of the avatar 100 determined by the emotion determination unit 242.

In step S1708, the memory control unit 248 determines whether the total value of the intensities is equal to or greater than a threshold. If the total value of the intensities is less than the threshold, the process ends without storing data including the behavior of the user 10 in the history data 232. On the other hand, if the total value of the intensities is equal to or greater than the threshold, the process proceeds to step S1709.

In step S1709, a behavior determined by the behavior determination unit 246, information analyzed by the sensor module unit 220 from the present time to a certain period in the past, and a state of the user 10 recognized by the state recognition unit 240 are stored in the history data 232.

As described above, the control device 200 includes a recognition unit (for example, sensor unit 210, sensor module unit 220, state recognition unit 240), an emotion estimation unit (for example, emotion determination unit 242), and an avatar control unit (for example, behavior determination unit 246, behavior control unit 250). The recognition unit recognizes a state of a child. The emotion estimation unit estimates the child's emotions based on the state of the child recognized by the recognition unit. The avatar control unit controls the avatar 100 to provide support according to the child's emotions estimated by the emotion estimation unit. As a result, the control device 200 can support and assist users (parents) raising children via the avatar 100 according to the child's state.

The avatar control unit may also control the avatar 100 to provide support for parents. As a result, the control device 200 can support parents via the avatar 100.

The avatar control unit may also propose methods for responding to child's emotions to parents. As a result, parents can respond to child's emotions and cope with parenting concerns using the proposed methods.

The control device 200 may also include a physical condition estimation unit 249. The physical condition estimation unit 249 estimates a child's physical condition based on a state of a child recognized by the recognition unit. The avatar control unit provides support according to a child's physical condition estimated by the physical condition estimation unit 249. As a result, the control device 200 can support and assist users (parents) according to a child's physical condition.

The recognition unit may also recognize a state of a child of the user 10 who communicates with the avatar 100. The avatar control unit may display a child avatar and control the child avatar to express at least one of the child's state and emotions. As a result, the control device 200 can notify the user 10 (the parent) of at least one of the child's state and emotions through a child avatar and support the user 10.

The present invention has been described using embodiments, but the technical scope of the present invention is not limited to a range described in the above embodiments. It will be apparent to those skilled in the art that various modifications and improvements can be made to the above embodiments. Such modified or improved embodiments are also included in the technical scope of the present invention, as is clear from the description of the claims. The above embodiments and modifications can be combined as appropriate.

The order of execution of each process, operation, step, and stage in the apparatus, system, program, and method shown in the claims, specification, and drawings is not necessarily limited unless explicitly stated as “before,” “prior to,” etc., or unless the output of a previous process is used in a subsequent process. Even if “first,” “next,” etc. are used for convenience in the operation flow in the claims, specification, and drawings, this does not mean that the operations must be performed in that order.

What is claimed is:

1. An information processing system comprising:
an image sensor configured to capture image frames of a user;
a communication interface configured to establish a secure connection with a remote server;
a storage device storing:
enrolled biometric templates associated with authorized users, and
response rule data; and
circuitry configured to:
extract facial feature data from the image frames,
compare the facial feature data against the enrolled biometric templates to authenticate the user,
in response to successful authentication, transmit a query including identification information of the authenticated user to the remote server via the communication interface,
receive, from the remote server via the secure connection, user-associated data corresponding to the identification information,
store the received user-associated data in the storage device,
compute a state value of the user based on sensor data,
determine, based on the state value and the user-associated data, an output action according to the response rule data, and
drive a display to render an avatar performing the determined output action.

2. The information processing system of claim 1, wherein the user-associated data includes information related to health and medical care of at least one of the user and a family member of the user.

3. The information processing system of claim 2, wherein the storage device further stores support rules defining actions to be supported by the avatar according to a health status and medical condition of the user or the family member, and wherein the output action is determined based on the support rules.

4. The information processing system of claim 3, wherein the support rules store response content and support content for at least one of:
emotional issues and methods for coping with concerns during pregnancy,
emotional issues and methods for coping with stress after childbirth and information related to childcare,
care content and methods for coping with end-of-life care for terminally ill patients and their families,
care content and rehabilitation for patients who require hospitalization, and emotional care, vital signs, and medication for patients with chronic diseases.

5. The information processing system of claim 2, wherein the user-associated data is acquired from at least one of an IoT device owned by the user or the family member and a medical institution system.

6. The information processing system of claim 2, wherein the circuitry is further configured to identify an issue from a speech content of the user, and wherein the output action is determined based on the identified issue and the user-associated data according to the response rule data.

7. The information processing system of claim 1, wherein the circuitry is further configured to recognize a mental state of the user, and wherein the output action includes conducting a conversation with the user and controlling the avatar to provide care according to the recognized mental state.

8. The information processing system of claim 7, wherein the circuitry is further configured to:
recognize that the user is exhibiting a negative emotion including at least one of sorrow, discomfort, anxiety, sadness, worry, and emptiness; and
control the avatar to provide a relaxation activity to the user in response to the recognized negative emotion.

9. The information processing system of claim 7, wherein the circuitry is further configured to control the avatar to support a daily rhythm of the user by providing prompts at predetermined times for at least one of:
measuring body temperature, blood pressure, or weight,
exercising or walking, and
waking up or preparing for sleep.

10. The information processing system of claim 7, wherein the circuitry is further configured to provide health management advice to the user based on trends in body temperature, blood pressure, or weight of the user over a predetermined period.

11. The information processing system of claim 1, wherein the circuitry is further configured to:
analyze an emotion of the user and an emotion of another user; and
determine a behavior of the avatar corresponding to the emotions of the user and the other user.

12. The information processing system of claim 11, wherein the circuitry is further configured to control a controlled object based on the determined behavior of the avatar.

13. The information processing system of claim 11, wherein the circuitry is further configured to determine an action to improve a relationship between the user and the other user based on the emotions of the user and the other user.

14. The information processing system of claim 11, wherein the circuitry is further configured to determine an action to make at least one of the user and the other user understand an emotion of the other, when one of the user and the other user is unaware of the other's emotion.

15. The information processing system of claim 1, wherein the circuitry is further configured to compute an avatar emotion value representing an emotional state of the avatar, the avatar emotion value being distinct from the state value of the user, and wherein the output action is determined based on a combination of the state value and the avatar emotion value according to the response rule data.

16. The information processing system of claim 15, wherein the circuitry is further configured to change the avatar emotion value based on a reaction of the user to executed behavior, wherein when the reaction is not poor, a joy emotion value of the avatar is increased, and when the reaction is poor, a sorrow emotion value of the avatar is increased.

17. The information processing system of claim 1, wherein the circuitry is further configured to:
determine whether a total intensity value based on a sum of avatar emotion values, a gesture intensity, and a speech content intensity equals or exceeds a threshold; and
store data including behavior of the user in history data when the total intensity value equals or exceeds the threshold, the stored data including information analyzed by a sensor subsystem from a present time to a past period.

18. An information processing system comprising:
an image sensor configured to capture image frames of a user;
a microphone configured to capture audio signals including utterances of the user;
a communication interface configured to communicate with a remote server via a network;
a storage device storing:
a person database including face images and attribute information of users,
response rule data defining avatar behaviors, and
support rule data defining actions to be supported according to a health status of the user or a family member of the user; and
circuitry configured to:
recognize the user by matching a face image captured by the image sensor against the person database,
transmit identification information of the recognized user to the remote server via the communication interface,
receive, from the remote server, information related to health and medical care of at least one of the user and a family member of the user,
compute an emotion value of the user based on data from the image sensor and the microphone,
determine a behavior of an avatar based on the emotion value, the received information related to health and medical care, and the support rule data,
recognize a mental state of the user, and based on the recognized mental state, control the avatar to provide care including at least one of mental care through conversation, daily life support, and health management advice, and
drive a display to render the avatar performing the determined behavior.

19. The information processing system of claim 18, wherein the circuitry is further configured to analyze an emotion of the user and an emotion of another user, determine a behavior of the avatar corresponding to the emotions of the user and the other user, and control a controlled object based on the determined behavior.

20. A method of processing information via an information processing system having an image sensor, a communication interface, a storage device storing enrolled biometric templates and response rule data, and circuitry, the method comprising:
capturing, by the image sensor, image frames of a user;
extracting, by the circuitry, facial feature data from the image frames;
comparing, by the circuitry, the facial feature data against the enrolled biometric templates to authenticate the user;
in response to successful authentication, transmitting, by the circuitry, a query including identification information of the authenticated user to a remote server via the communication interface;
receiving, from the remote server, user-associated data corresponding to the identification information;
computing, by the circuitry, a state value of the user based on sensor data;
determining, by the circuitry, based on the state value and the user-associated data, an output action according to the response rule data; and
driving, by the circuitry, a display to render an avatar performing the determined output action.
